(19) United States
(12) Patent Application Publication
(10) Pub. No.: US 2009/0215743 A1
(43) Pub. Date: Aug. 27, 2009

(54) Tricyclic benzazepine derivatives and their use

(75) Inventors: Elisabeth Woltering, Hilden (DE); Michael Härter, Leverkusen (DE); Timo Flessner, Wuppertal (DE); Mario Jeske, Solingen (DE); Nils Griebenow, Dormagen (DE); Frank Süssmeier, Wuppertal (DE); Hilmar Bischoff, Wuppertal (DE); Martin Raabe, Ulm (DE); Anja Buchmüller, Essen (DE); Peter Kolkhof, Wuppertal (DE); Peter Ellinghaus, Wuppertal (DE); Olaf Weber, Wülfrath (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 10/586,023

(22) PCT Filed: Dec. 31, 2004
(86) PCT No.: PCT/EP2004/014871

§ 371 Date: Feb. 17, 2009

(30) Foreign Application Priority Data
Jan. 14, 2004 (DE) 102004001871.5

(51) Int. Cl. A61K 31/5383; C07D 498/04

(52) U.S. Cl. 514/211.12; 540/548

(57) ABSTRACT

The present application relates to novel tricyclic benzazepine derivatives, processes for their preparation, their use for the treatment and/or prophylaxis of diseases, and their use for producing medicaments for the treatment and/or prophylaxis of diseases, preferably for the treatment and/or prevention of cardiovascular disorders, especially of dyslipidaemias, arteriosclerosis, restenosis and ischaemias.

The present application relates to novel tricyclic benzazepine derivatives, processes for their preparation, their use for the treatment and/or prophylaxis of diseases, and their use for producing medicaments for the treatment and/or prophylaxis of diseases, preferably for the treatment and/or prevention of cardiovascular disorders, especially of dyslipidaemias, arteriosclerosis, restenosis and ischaemias.

A large number of epidemiological studies has shown a causal connection between dyslipidaemias and cardiovascular disorders. Elevated plasma cholesterol in isolation is one of the greatest risk factors for cardiovascular disorders such as, for example, arteriosclerosis. This relates both to an isolated hypercholesterolaemia and to hypercholesterolaemias combined with, for example, elevated plasma triglycerides or low plasma HDL-cholesterol. Substances which have a cholesterol- or combined cholesterol- and triglyceride-lowering effect ought therefore to be suitable for the treatment and prevention of cardiovascular disorders.

It has already been shown in animal models that plasma cholesterol and triglycerides are lowered by squalene synthase inhibitors. Squalene synthase (EC 2.5.1.21) catalyses the conversion, by reductive condensation, of farnesyl pyrophosphate into squalene. This is a crucial step in cholesterol biosynthesis. Whereas farnesyl pyrophosphate and precursors are also of importance for other cellular metabolic pathways and reactions, squalene serves exclusively as precursor for cholesterol. Inhibition of squalene synthase thus leads directly to a reduction in cholesterol biosynthesis and thus to a fall in plasma cholesterol levels. It has additionally been shown that squalene synthase inhibitors also reduce plasma triglyceride levels. Inhibitors of squalene synthase might thus be employed for the treatment and/or prevention of cardiovascular disorders such as, for example, dyslipidaemias, arteriosclerosis, ischaemia/reperfusion, restenosis and arterial inflammations [cf., for example, Eur. Heart J. 19 (Suppl. A), A2-A11 (1998); Prog. Med. Chem. 33, 331-378 (1996); Europ. J. Pharm. 431, 345-352 (2001)].

It was an object of the present invention to provide novel compounds which can be employed as squalene synthase inhibitors for the treatment and/or prevention in particular of cardiovascular disorders.

Benzoxazepines having CNS activity are claimed in U.S. Pat. No. 4,374,842 and U.S. Pat. No. 4,476,133. U.S. Pat. No. 3,812,259 describes benzodiazepine derivatives as addition to animal feed. The use of certain azepine derivatives for controlling the levels of lipoproteins in blood plasma is claimed in EP 875 247. Triazolooxazepines for the treatment of inflammatory states and allergies are disclosed in JP 05 345 785. EP 638 560 claims the use of azepine derivatives for the treatment of osteoporosis.

The present invention relates to compounds of the general formula (I)

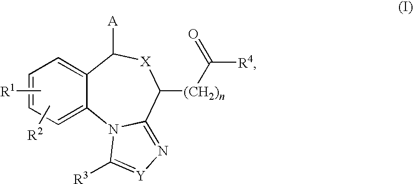

in which
A is (C₆-C₁₀)-aryl or 5- to 10-membered heteroaryl, each of which may be substituted up to three times, identically or differently, by substituents selected from the group of halogen, cyano, nitro, trifluoromethyl, hydroxy, fluoromethoxy, trifluoromethoxy, (C₁-C₆)-alkyl, (C₁-C₆)-alkoxy, amino, mono- and di-(C₁-C₆)-alkylamino,
   or
   is a group of the formula

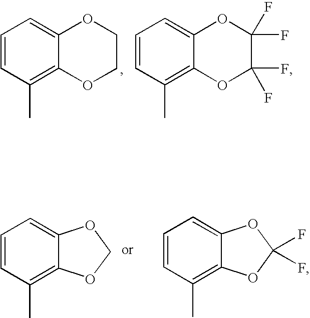

X is O, S or N—R⁵ in which
   R⁵ is hydrogen or (C₁-C₆)-alkyl,
Y is N or C—RF⁶ in which
   R⁶ is hydrogen, hydroxy or (C₁-C₆)-alkyl,
n is the number 1, 2 or 3,
R¹ and R² are identical or different and are independently of one another hydrogen, halogen, cyano, nitro, trifluoromethyl, trifluoromethoxy, (C₁-C₆)-alkyl or (C₁-C₆)-alkoxy,
R³ is (C₁-C₈)-alkyl, (C₂-C₈)-alkenyl, (C₂-C₈)-alkynyl, each of which may be substituted by (C₃-C₈)-cycloalkyl, or is (C₃-C₈)-cycloalkyl, where
   (C₁-C₈)-alkyl, (C₂-C₈)-alkenyl, (C₂-C₈)-alkynyl and (C₃-C₈)-cycloalkyl may each be substituted by hydroxy, (C₁-C₆)-alkoxy, (C₂-C₆)-alkenoxy, (C₁-C₆)-acyloxy, amino, mono- or di-(C₁-C₆)-alkylamino or by a 4- to 8-membered saturated heterocycle which is linked via an N atom and which may comprise a further heteroatom from the series O or S,
and
R⁴ is a group of the formula —OR⁷ or —NR⁸R⁹ in which
   R⁷ is hydrogen or (C₁-C₆)-alkyl,
   R⁸ and R⁹ are identical or different and are independently of one another hydrogen, (C₁-C₆)-alkyl or (C₃-C₈)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
   or
   R⁸ and R⁹ form together with the nitrogen atom to which they are bonded a 4- to 8-membered heterocycle which may comprise a further ring heteroatom from the series N—R¹⁰, O, S, SO or SO₂ and may be substituted by substituents selected from the group of hydroxy, oxo, amino, (C₁-C₆)-alkyl, carboxyl, (C₁-C₆)-alkoxycarbonyl, aminocarbonyl, mono- and di-(C₁-C₆)-alkylaminocarbonyl, in which
      (C₁-C₆)-alkyl in turn may be substituted by substituents selected from the group of hydroxy, amino, carboxyl, (C₁-C₆)-alkoxycarbonyl, aminocarbonyl, mono- and di-(C₁-C₆)-alkylaminocarbonyl,
      and
      R¹⁰ is hydrogen, (C₁-C₄)-alkyl, (C₁-C₄)-acyl or (C₁-C₄)-alkoxycarbonyl in which
         (C₁-C₄)-alkyl may in turn be substituted by carboxyl or (C₁-C₄)-alkoxycarbonyl,
            and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds of the invention can occur in tautomeric forms, the present invention encompasses all tautomeric forms.

Salts preferred for the purposes of the present invention are physiologically acceptable salts of the compounds of the invention. However, salts which are themselves unsuitable for pharmaceutical applications but can be used for example for isolating or purifying the compounds of the invention are also encompassed.

Physiologically acceptable salts of the compounds of the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer for the purposes of the invention to those forms of the compounds of the invention which form a complex in the solid or liquid state through coordination with solvent molecules. Hydrates are a specific form of solvates in which the coordination takes place with water. Solvates preferred in the context of the present invention are hydrates.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

In the context of the present invention, the substituents have the following meaning unless otherwise specified:

(C₁-C₈)-Alkyl, (C₁-C₆)-alkyl and (C₁-C₄)-alkyl are in the context of the invention a straight-chain or branched alkyl radical having respectively 1 to 8, 1 to 6 and 1 to 4 carbon atoms. A straight-chain or branched alkyl radical having 1 to 6 or 1 to 4 carbon atoms is preferred. A straight-chain or branched alkyl radical having 1 to 4 carbon atoms is particularly preferred. Examples which may be preferably mentioned are: methyl, ethyl, n-propyl, isopropyl, n-butyl, iso-butyl, sec-butyl, tert-butyl, 1-ethylpropyl, n-pentyl and n-hexyl.

(C₂-C₈)-Alkenyl in the context of the invention is a straight-chain or branched alkenyl radical having 2 to 8 carbon atoms. A straight-chain or branched alkenyl radical having 2 to 6 carbon atoms is preferred, particularly preferably having 2 to 4 carbon atoms. Examples which may be preferably mentioned are: vinyl, allyl, isopropenyl and n-but-2-en-1-yl.

(C₂-C₈)-Alkynyl in the context of the invention is a straight-chain or branched alkynyl radical having 2 to 8 carbon atoms. A straight-chain or branched alkynyl radical having 2 to 6 carbon atoms is preferred, particularly preferably having 2 to 4 carbon atoms. Examples which may be preferably mentioned are: ethynyl, n-prop-2-yn-1-yl and n-but-2-yn-1-yl.

(C₃-C₈)-Cycloalkyl and (C₁-C₆)-cycloalkyl in the context of the invention are a monocyclic cycloalkyl group having respectively 3 to 8 and 3 to 6 carbon atoms. A cycloalkyl radical having 3 to 6 carbon atoms is preferred. Examples which may be preferably mentioned are: cyclopropyl, cyclobutyl, cyclopentyl, cyclohexyl and cycloheptyl.

(C₆-C₁₀)-Aryl in the context of the invention is an aromatic radical having preferably 6 to 10 carbon atoms. Preferred aryl radicals are phenyl and naphthyl.

(C₁-C₆)-Alkoxy and (C₁-C₄)-alkoxy in the context of the invention are a straight-chain or branched alkoxy radical having respectively 1 to 6 and 1 to 4 carbon atoms. A straight-chain or branched alkoxy radical having 1 to 4 carbon atoms is preferred. Examples which may be preferably mentioned are: methoxy, ethoxy, n-propoxy, isopropoxy and tert-butoxy.

(C₂-C₆)-Alkenoxy in the context of the invention is a straight-chain or branched alkenoxy radical having 2 to 6 carbon atoms. A straight-chain or branched alkenoxy radical having 2 to 4 carbon atoms is preferred. Examples which may be preferably mentioned are: allyloxy, isopropenyloxy, 2-methylprop-2-en-1-yloxy, n-but-2-en-1-yloxy and n-but-3-en-1-yloxy.

(C₁-C₆)-alkoxycarbonyl and (C₁-C₄)-alkoxycarbonyl in the context of the invention are a straight-chain or branched alkoxy radical having respectively 1 to 6 and 1 to 4 carbon atoms which is linked via a carbonyl group. A straight-chain or branched alkoxycarbonyl radical having 1 to 4 carbon atoms in the alkoxy group is preferred. Examples which may be preferably mentioned are: methoxycarbonyl, ethoxycarbonyl, n-propoxycarbonyl, isopropoxycarbonyl and tert-butoxycarbonyl.

Mono-(C₁-C₆)-alkylamino and mono-(C₁-C₄)-alkylamino in the context of the invention are an amino group having a straight-chain or branched alkyl substituent which has respectively 1 to 6 and 1 to 4 carbon atoms. A straight-chain or branched monoalkylamino radical having 1 to 4 carbon atoms is preferred. Examples which may be preferably mentioned are: methylamino, ethylamino, n-propylamino, isopropylamino and tert-butylamino.

Di-(C₁-C₆)-alkylamino and di-(C₁-C₄)-alkylamino in the context of the invention are an amino group having two identical or different straight-chain or branched alkyl substituents which each have respectively 1 to 6 and 1 to 4 carbon atoms. Straight-chain or branched dialkylamino radicals having in each case 1 to 4 carbon atoms are preferred. Examples which may be preferably mentioned are: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Mono- or di-(C₁-C₆)-alkylaminocarbonyl and mono- or di-(C₁-C₄)-alkylaminocarbonyl in the context of the invention are an amino group which is linked via a carbonyl group and which has respectively a straight-chain or branched and two identical or different straight-chain or branched alkyl substituents each having respectively 1 to 6 and 1 to 4 carbon atoms. Examples which may be preferably mentioned are: methylaminocarbonyl, ethylaminocarbonyl, isopropylaminocarbonyl, tert-butylaminocarbonyl, N,N-dimethylaminocarbonyl, N,N-diethylaminocarbonyl, N-ethyl-N-methylaminocarbonyl and N-tert-butyl-N-methylaminocarbonyl.

(C₁-C₄)Acyl [(C₁-C₄)-alkanoyl] in the context of the invention is a straight-chain or branched alkyl radical having 1 to 4 carbon atoms which has a doubly bonded oxygen atom in position 1 and is linked via position 1. Examples which may be preferably mentioned are: formyl, acetyl, propionyl, n-butyryl and iso-butyryl.

(C₁-C₆)-Acyloxy in the context of the invention is a straight-chain or branched alkyl radical having 1 to 6 carbon atoms which has a doubly bonded oxygen atom in position 1 and is linked via a further oxygen atom in position 1. An acyloxy radical having 1 to 4 carbon atoms is preferred. Examples which may be preferably mentioned are: acetoxy, propionoxy, n-butyroxy, i-butyroxy, pivaloyloxy and n-hexanoyloxy.

5- to 10-membered heteroaryl in the context of the invention is a mono- or, where appropriate, bicyclic aromatic heterocycle (heteroaromatic system) having up to three identical or different heteroatoms from the series N, O and/or S, which is linked via a ring carbon atom or, where appropriate, via a ring nitrogen atom of the heteroaromatic system. Examples which may be mentioned are: furanyl, pyrrolyl, thienyl, pyrazolyl, imidazolyl, thiazolyl, oxazolyl, isoxazolyl, isothiazolyl, pyridyl, pyrimidinyl, pyridazinyl, pyrazinyl, benzofuranyl, benzothienyl, benzimidazolyl, benzoxazolyl, indolyl, indazolyl, quinolinyl, isoquinolinyl, naphthyridinyl, quinazolinyl, quinoxalinyl. 5- to 6-membered heteroaryl radicals having up to two heteroatoms from the series N, O and/or S are preferred, such as, for example, furyl, thienyl, thiazolyl, oxazolyl, isothiazolyl, isoxazolyl, imidazolyl, pyridyl, pyrimidinyl, pyridazinyl, pyrazinyl.

A 4- to 8-, 5- to 7- and 5- to 6-membered heterocycle in the context of the invention is a saturated or partially unsaturated heterocycle having respectively 4 to 8, 5 to 7 and 5 to 6 ring atoms which comprises a ring nitrogen atom, is linked via the latter and may comprise a further heteroatom from the series N, O, S, SO or SO₂. A 5- to 7-membered saturated, N-linked heterocycle which may comprise a further heteroatom from the series N, O or S is preferred. Examples which may be mentioned are: pyrrolidinyl, pyrrolinyl, thiazolidinyl, piperidinyl, piperazinyl, morpholinyl, thiomorpholinyl, azepinyl, 1,4-diazepinyl. Piperidinyl, piperazinyl, morpholinyl, thiomorpholinyl, pyrrolidinyl and thiazolidinyl are particularly preferred.

Halogen in the context of the invention includes fluorine, chlorine, bromine and iodine. Chlorine or fluorine are preferred.

If radicals in the compounds according to the invention are substituted, the radicals may, unless otherwise specified, be substituted one or more times. In the context of the present invention, all radicals which occur more than once have a mutually independent meaning. Substitution by one, two or three identical or different substituents is preferred. Substitution by one substituent is very particularly preferred.

Preference is given to compounds of the formula (I) in which
A is phenyl, naphthyl or pyridyl, each of which may be substituted up to twice, identically or differently, by substituents selected from the group of fluorine, chlorine, bromine, cyano, nitro, trifluoromethyl, fluoromethoxy, trifluoromethoxy, (C₁-C₄)-alkyl, (C₁-C₄)-alkoxy, amino, mono- and di-(C₁-C₄)-alkylamino,
   or
   is a group of the formula

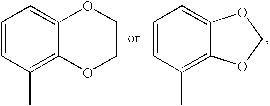

[change oder to or]
X is O,
Y is N or C—R⁶ in which
   R⁶ is hydrogen, hydroxy or (C₁-C₄)-alkyl,
n is the number 1, 2 or 3,
R¹ and R² are identical or different and are independently of one another hydrogen, fluorine, chlorine, bromine, cyano, nitro, trifluoromethyl, trifluoromethoxy, (C₁-C₄)-alkyl or (C₁-C₄)-alkoxy,
R³ is (C₁-C₆)-alkyl which may be substituted by (C₃-C₆)-cycloalkyl, or is (C₃-C₆)-cycloalkyl, where
   (C₁-C₆)-alkyl and (C₃-C₆)-cycloalkyl may each be substituted by hydroxy, (C₁-C₄)-alkoxy or amino,
and
R⁴ is a group of the formula —OR⁷ or —NR⁸R⁹, in which
   R⁷ is hydrogen or (C₁-C₆)-alkyl,
   R⁸ and R⁹ are identical or different and are independently of one another hydrogen, (C₁-C₆)-alkyl or (C₃-C₆)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
   or
   R⁸ and R⁹ form together with the nitrogen atom to which they are bonded a 5- to 7-membered heterocycle which may comprise a further ring heteroatom from the series N—R¹⁰, O, S or SO₂ and may be substituted by substituents selected from the group of hydroxy, oxo, amino, (C₁-C₆)-alkyl, carboxyl, (C₁-C₆)-alkoxycarbonyl, aminocarbonyl, mono- and di-(C₁-C₆)-alkylaminocarbonyl, in which
      (C₁-C₆)-alkyl in turn may be substituted by substituents selected from the group of hydroxy, amino, carboxyl, (C₁-C₆)-alkoxycarbonyl, aminocarbonyl, mono- and di-(C₁-C₆)-alkylaminocarbonyl,
      and
      R¹⁰ is hydrogen, (C₁-C₄)-alkyl, (C₁-C₄)-acyl or (C₁-C₄)-alkoxycarbonyl in which
         (C₁-C₄)-alkyl in turn may be substituted by carboxyl or (C₁-C₄)-alkoxycarbonyl,
            and the salts, solvates and solvates of the salts thereof.

Particular preference is given to compounds of the formula (I) in which
A is phenyl which is substituted once or twice, identically or differently, by fluorine, chlorine, bromine, methyl, methoxy, ethoxy, fluoromethoxy or dimethylamino,
X is O,
y is N,
n is the number 1,
R¹ and R² are independently of one another hydrogen or chlorine,
R³ is (C₁-C₆)-alkyl or (C₃-C₆)-cycloalkyl, each of which may be substituted by hydroxy, (C₁-C₄)-alkoxy or amino,
and
R⁴ is a group of the formula —OR⁷ or —NR⁸R⁹ in which
   R⁷ is hydrogen or (C₁-C₄)-alkyl,
   R⁸ and R⁹ are identical or different and are independently of one another hydrogen or (C₁-C₄)-alkyl which may be substituted by carboxyl or (C₁-C₄)-alkoxycarbonyl,
   or
   R⁸ and R⁹ form together with the nitrogen atom to which they are bonded a 5- or 6-membered heterocycle which may comprise a further ring heteroatom from the series N—R¹⁰, O, S or SO₂ and may be substituted by substituents selected from the group of hydroxy, oxo, amino, (C₁-C₄)-alkyl, carboxyl, (C₁-C₄)-alkoxycarbonyl, aminocarbonyl, mono- and di-(C₁-C₄)-alkylaminocarbonyl, in which
      (C₁-C₄)-alkyl in turn may be substituted by substituents selected from the group of hydroxy, amino, carboxyl, (C₁-C₄)-alkoxycarbonyl, aminocarbonyl, mono- and di-(C₁-C₄)-alkylaminocarbonyl,
      and
      R¹⁰ is hydrogen, (C₁-C₄)-alkyl or (C₁-C₄)-acyl,
         and the salts, solvates and solvates of the salts thereof.

Of particular importance are compounds of the general formula (I-A)

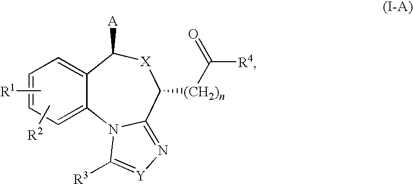

in which
A, X, Y, n, R¹, R², R³ and R⁴ each have the meanings indicated above,
and the salts, solvates and solvates of the salts thereof.

Of very particular importance are compounds of the general formula (I-B)

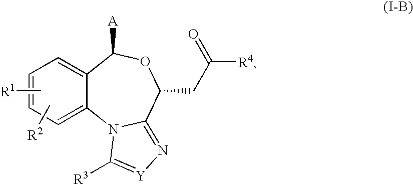

in which
A, Y, R¹, R², R³ and R⁴ each have the meanings indicated above,
and the salts, solvates and solvates of the salts thereof.

The definitions of radicals indicated specifically in the respective combinations or preferred combinations of radicals are replaced as desired irrespective of the particular combinations indicated for the radicals also by the definitions of radicals of other combinations.

Combinations of two or more of the abovementioned preferred ranges are very particularly preferred.

The invention further relates to a process for preparing the compounds according to the invention, characterized in that compounds of the formula (II)

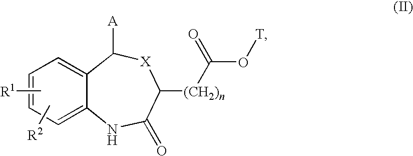

in which R¹, R², A, X and n each have the abovementioned meanings, and
T is (C₁-C₄)-alkyl,
   are firstly converted in an inert solvent with a suitable sulphurizing agent such as, for example, diphosphorus pentasulphide into compounds of the formula (III)

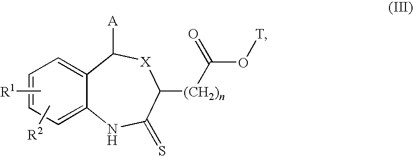

in which R¹, R², A, T, X and n each have the abovementioned meanings,
[cf., for example, Ma et al., Tetrahedron Lett. 41 (12), 1947-1950 (2000)], subsequently reacted in an inert solvent with a compound of the formula (IV)

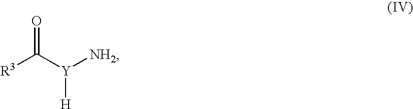

in which Y and R³ each have the abovementioned meanings,
with cyclization to give compounds of the formula (V)

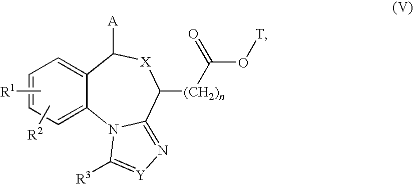

in which R¹, R², R³, A, T, X, Y and n each have the abovementioned meanings,
[cf., for example, Weber et al., Justus Liebigs Ann. Chem., 1250-1256 (1978)], the latter are hydrolysed under acidic conditions to carboxylic acids of the formula (VI)

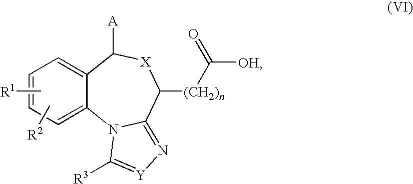

in which R¹, R², R³, A, X, Y and n each have the abovementioned meanings,
and then converted by methods known from the literature for esterification and amidation of carboxylic acids into the compounds of the formula (I)
and the compounds of the formula (I) are where appropriate reacted with the appropriate (i) solvents and/or (ii) bases or acids to give the solvates, salts and/or solvates of the salts thereof.

Examples of inert solvents for process step (II)→(III) are ethers such as diethyl ether, dioxane, tetrahydrofuran, glycol dimethyl ether or diethylene glycol dimethyl ether, or hydrocarbons such as benzene, xylene, toluene, hexane, cyclohexane or petroleum fractions. It is likewise possible to employ mixtures of the said solvents. Glycol dimethyl ether (1,2-dimethoxyethane) is preferred.

The sulphurizing agent preferably used is diphosphorus pentasulphide, which is employed in an amount of from 1 to 1.5 mol based on 1 mol of the compound of the formula (II). The reaction is preferably carried out in the presence of from 1 to 2 equivalents of sodium bicarbonate based on the compound of the formula (II).

The reaction generally takes place in a temperature range from +20° C. to +150° C., preferably from +50° C. to +100° C. The reaction can be carried out under atmospheric, elevated or reduced pressure (e.g. from 0.5 to 5 bar). It is generally carried out under atmospheric pressure.

Examples of inert solvents for process step (III+(IV)→(V) are ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, or dipolar aprotic solvents such as dimethylformamide, dimethyl sulphoxide or acetonitrile. It is likewise possible to employ mixtures of the said solvents. Dioxane is preferred.

The compound of the formula (IV) is in this case employed in an amount of from 1.5 to 10 mol, preferably from 2 to 5 mol, based on 1 mol of the compound of the formula (M). The reaction generally takes place in a temperature range from +20° C. to +150° C., preferably from +80° C. to +120° C. The reaction can be carried out under atmospheric, elevated or reduced pressure (e.g. from 0.5 to 5 bar). It is generally carried out under atmospheric pressure.

Examples of inert solvents for process step (V)→(VI) are ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, or dipolar aprotic solvents such as acetone, dimethylformamide, dimethyl sulphoxide or acetonitrile, or else water. It is likewise possible to employ mixtures of the said solvents. Ethanol/water is preferred.

Suitable acids are aqueous solutions of the usual inorganic acids such as, for example, hydrochloric acid, sulphuric acid, phosphoric acid or hydrobromic acid. Hydrochloric acid is preferred. The reaction generally takes place in a temperature range from +20° C. to +150° C., preferably from +50° C. to +100° C. The reaction can be carried out under atmospheric, elevated or reduced pressure (e.g. from 0.5 to 5 bar). It is generally carried out under atmospheric pressure.

Process step (VI)→(I) is carried out by methods known from the literature for the esterification or amidation (amide formation) of carboxylic acids.

Examples of inert solvents for an amidation in process step (VI)→(I) are ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloromethane, tetrachloromethane, 1,2-dichloroethane, trichloroethylene or chlorobenzene, or other solvents such as ethyl acetate, pyridine, dimethyl sulphoxide, dimethylformamide, N,N′-dimethylpropyleneurea (DMPU), N-methylpyrrolidone (NMP), acetonitrile or acetone. It is likewise possible to use mixtures of the said solvents. Tetrahydrofuran, dimethylformamide or mixtures of these two solvents are preferred.

Examples of suitable condensing agents for an amide formation in process step (VI)→(I) are carbodiimides such as N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC), or phosgene derivatives such as N,N′-carbonyldiimidazole, or 1,2-oxazolium compounds such as 2-ethyl-5-phenyl-1,2-oxazolium-3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, isobutyl chloroformate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), where appropriate combined with further auxiliaries such as 1-hydroxybenzotriazole or N-hydroxysuccinimide, and as bases alkali metal carbonates, e.g. sodium or potassium carbonate or bicarbonate, or organic bases such as trialkylamines, e.g. triethylamine, N-methylmorpholine, N-methylpiperidine or N,N-diisopropylethylamine. PyBOP in combination with N,N-diisopropylethylamine is preferably used.

An amide formation in process step (VI)→(I) is generally carried out in a temperature range from 0° C. to +100° C., preferably from 0° C. to +40° C. The reaction can be carried out under atmospheric, elevated or reduced pressure (e.g. from 0.5 to 5 bar). It is generally carried out under atmospheric pressure.

The compounds of the formula (II) can be prepared in analogy to processes disclosed in the literature for example by firstly converting compounds of the formula (VII)

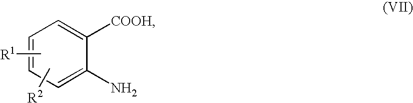

in which R¹ and R² each have the abovementioned meanings,
with acetic anhydride into benzoxazin-4-one derivatives of the formula (VIII)

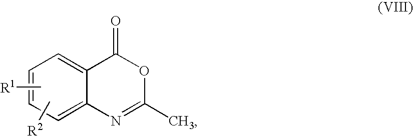

in which R¹ and R² each have the abovementioned meanings,
[cf., for example, Jiang et al., J. Med. Chem. 33 (6), 1721-1728 (1990)], then reacting the latter in an inert solvent with an organometallic compound of the formula (IX)

A-M (IX),

in which A has the abovementioned meaning, and
M is lithium or the Grignard residue —MgCl, —MgBr or —MgI,
and subsequent acidic hydrolysis to give compounds of the formula (X)

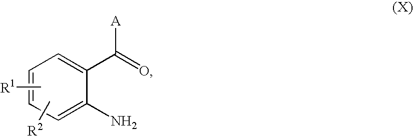

in which A, R¹ and R² each have the abovementioned meanings,
[cf., for example, Miki et al., Bioorg. Med. Chem. 10, 401-414 (2002)], subsequently converted by methods customary in the literature into compounds of the formula (XI)

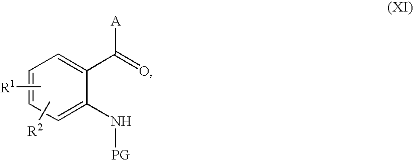

in which A, R¹ and R² each have the abovementioned meanings, and
PG is a suitable amino protective group such as preferably allyl,
the latter are then reacted in an inert solvent in the presence of a base with a compound of the formula (XII)

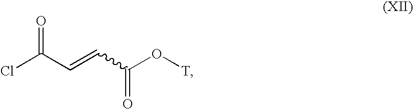

in which T has the abovementioned meaning,
to give compounds of the formula (XII)

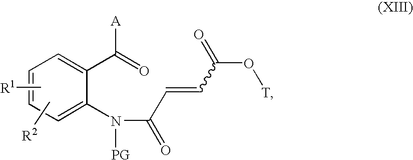

in which A, T, PG, R¹ and R² each have the abovementioned meanings,
[cf., for example, Miki et al., J. Med. Chem. 45 (20), 4571-4580 (2002)], and then reduced in an inert solvent with the aid of a borohydride such as, for example, sodium borohydride selectively to compounds of the formula (XIV)

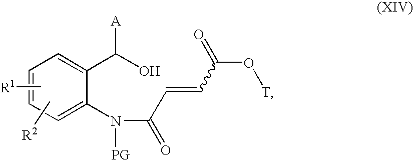

in which A, T, PG, R¹ and R² each have the abovementioned meanings,
[cf., for example, Miki et al., Bioorg. Med. Chem. 10, 401-414 (2002)], subsequently cyclized in an inert solvent in the presence of a base to compounds of the formula (XV)

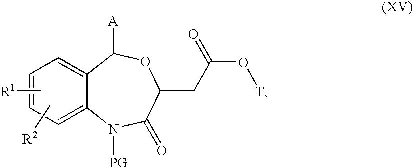

in which R¹, R², A, T and PG each have the abovementioned meanings,
[cf., for example, Miki et al., J. Med. Chem. 45 (20), 4571-4580 (2002)] and finally the amino protective group is eliminated again by methods customary in the literature. The cyclization (XIV)→(XV) may also occur wholly or partly in situ during the described borohydride reduction of the compound of the formula (XIII).

The compounds of the formulae (IV), (VII), (IX), (XII) are commercially available, disclosed in the literature or can be prepared in analogy to processes disclosed in the literature.

Compounds of the general formula (I) in which X is S or N—R⁵ can be prepared starting from compounds of the formula (XI), (XIII) or (XIV) by appropriate transformations, disclosed in the literature, of the carbonyl or hydroxy group and further reaction in analogy to the reaction sequence described above.

Compounds of the general formula (I) in which n is the number 2 or 3 can be prepared starting from compounds of the formula (II), (III), (V) or (VI) in which n is in each case the number 1 by methods disclosed in the literature for the homologization of carbonyl compounds (e.g. Arndt-Eistert, Wittig, Horner reaction) and further reaction in analogy to the reaction sequence described above. Compounds of the general formula (I) in which n is the number 3 can also be prepared starting from a compound of the formula (XI) by a reaction analogous to process step (XI)+(XII)→(XIII) with a compound of the formula (XVI)

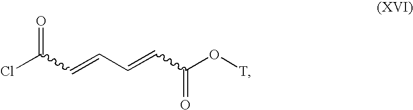

in which T has the abovementioned meaning,
subsequent reduction of the keto group [in analogy to (XIII)→(XIV)], cyclization by 1,5-addition onto the dienoate system, hydrogenation of the remaining double bond and further reaction in analogy to the reaction sequence described previously.

Preparation of the compounds according to the invention can be illustrated by the following synthesis schemes:

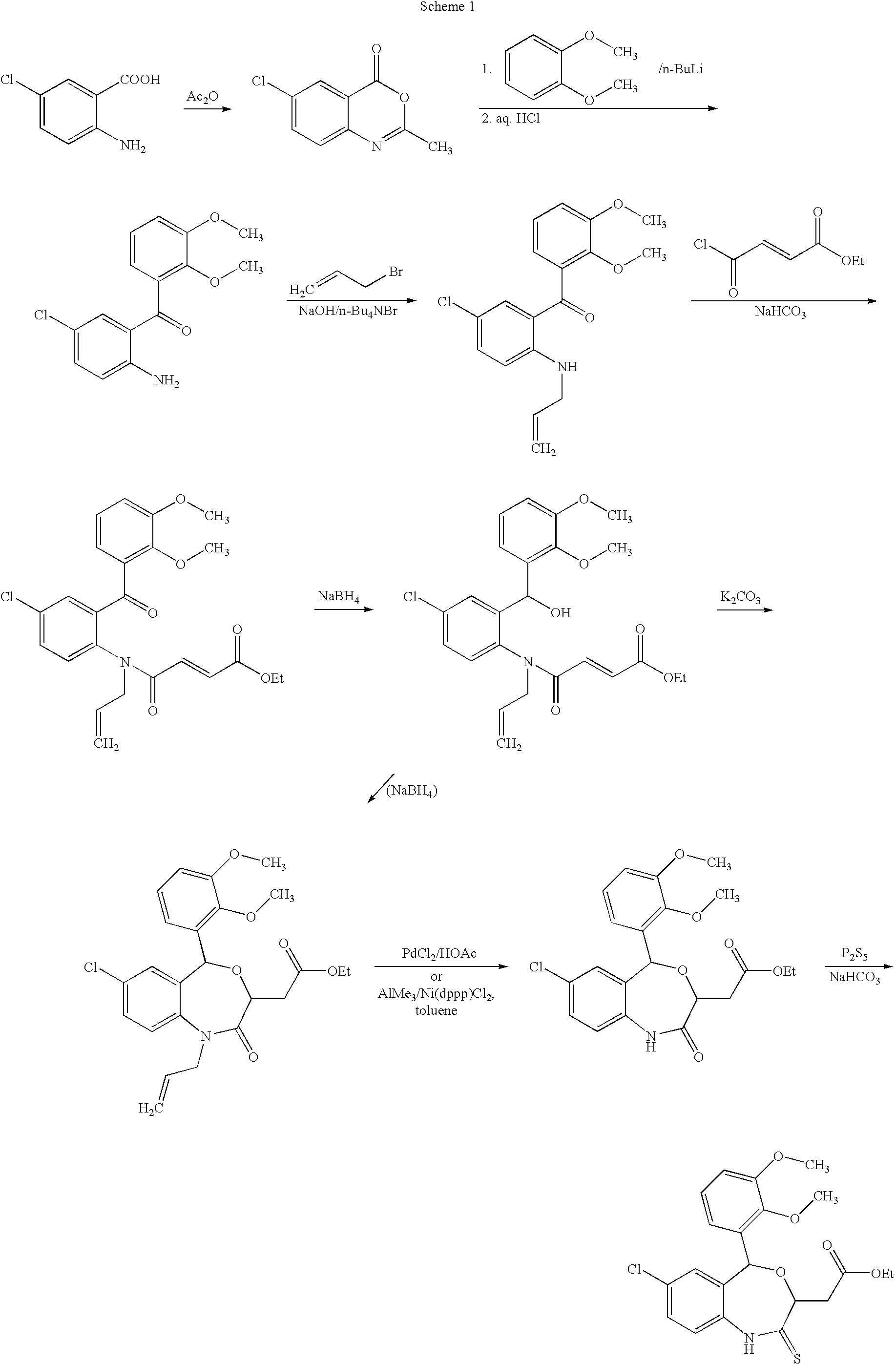

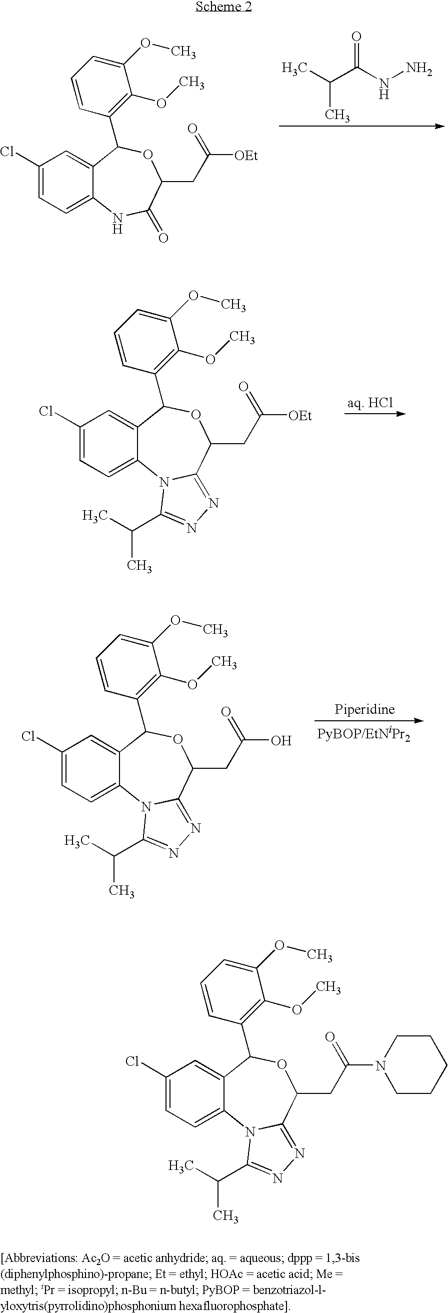

The compounds according to the invention have valuable pharmacological properties and can be used for the prevention and treatment of disorders in humans and animals. In particular, the compounds according to the invention are highly effective inhibitors of squalene synthase and inhibit cholesterol biosynthesis. The compounds according to the invention bring about a lowering of the cholesterol level and of the triglyceride level in the blood. They can therefore be employed for the treatment and prevention of cardiovascular disorders, in particular of hypolipoproteinaemia, dyslipidaemias, hyperlipidaemias or arteriosclerosis. The compounds according to the invention may additionally be used for the treatment and prevention of adiposity and corpulence (obesity). The compounds according to the invention are further suitable for the treatment and prevention of strokes and of Alzheimer's disease.

The present invention further relates to the use of the compounds according to the invention for the treatment and/or prophylaxis of disorders, in particular of the aforementioned disorders.

The present invention further relates to the use of the compounds according to the invention for producing a medicament for the treatment and/or prophylaxis of disorders, especially of the aforementioned disorders.

The present invention further relates to a method for the treatment and/or prophylaxis of disorders, in particular of the aforementioned disorders, using an effective amount of at least one of the compounds according to the invention.

The present invention further relates to medicaments comprising at least one compound according to the invention and at least one or more further active ingredients, in particular for the treatment and/or prophylaxis of the aforementioned disorders. Examples which may be preferably mentioned of active ingredients suitable for combination are: cholesterol-lowering statins, cholesterol absorption inhibitors, HDL-elevating or triglyceride-lowering and/or apolipoprotein B-lowering substances, oxidation inhibitors or compounds having antiinflammatory activity. Combinations with these active ingredients are preferably suitable for the treatment of dyslipidaemias, combined hyperlipidaemias, hypercholesterolaemias or hypertriglyceridaemias.

The said combinations can also be employed for the primary or secondary prevention of coronary heart disease (e.g. myocardial infarction) and for peripheral arterial disorders.

Examples of statins in the context of the invention are lovastatin, simvastatin, pravastatin, fluvastatin, atorvastatin, rosuvastatin and pitavastatin. Examples of cholesterol absorption inhibitors are cholestyramines or ezetimibe; examples of HDL-elevating or triglyceride-lowering or apolipoprotein B-lowering substances are fibrates, niacin, PPAR agonists, IBAT inhibitors, MTP inhibitors and CETP inhibitors. Compounds having antiinflammatory activity are, for example, aspirin.

The present invention further relates additionally to the combination of the compounds according to the invention with a glucosidase inhibitor and/or amylase inhibitor for the treatment of familial hyperlipidaemia, of adiposity (obesity) and of diabetes mellitus.

Examples of glucosidase inhibitors and/or amylase inhibitors in the context of the invention are acarbose, adiposins, voglibose, miglitol, emiglitates, MDL-25637, camiglibose (MDL-73945), tendamistats, AI-3688, trestatin, pradimicin Q and salbostatin. Combination of acarbose, miglitol, emiglitates or voglibose with one of the compounds according to the invention is preferred.

The compounds of the invention can act systemically and/or locally. For this purpose, they can be administered in a suitable way such as, for example, by the oral, parenteral, pulmonal, nasal, sublingual, lingual, buccal, rectal, dermal, transdermal, conjunctival, otic route or as implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds of the invention can be converted into the stated administration forms. This can take place in a manner known per se by mixing with inert, nontoxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colours (e.g. inorganic pigments such as, for example, iron oxides) and masking flavours and/or odours.

The present invention further relates to medicaments which comprise at least one compound according to the invention, normally together with one or more inert, nontoxic, pharmaceutically suitable excipients, and to the use thereof for the aforementioned purposes.

It has generally proved advantageous to administer on parenteral administration amounts of about 0.001 to 1 mg/kg, preferably about 0.01 to 0.5 mg/kg, of bodyweight to achieve effective results, and on oral administration the dosage is about 0.01 to 100 mg/kg, preferably about 0.01 to 20 mg/kg, and very particularly preferably 0.1 to 10 mg/kg, of bodyweight.

It may nevertheless be necessary where appropriate to deviate from the stated amounts, in particular as a function of the bodyweight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations

CI chemical ionization (in MS)
DCI direct chemical ionization (in MS)
DMSO dimethyl sulphoxide
EI electron impact ionization (in MS)
ESI electrospray ionization (in MS)
GC/MS coupled gas chromatography-mass spectroscopy
h hour(s)
HPLC high pressure, high performance liquid chromatography
LC/MS coupled liquid chromatography-mass spectroscopy
min. minute(s)
MS mass spectroscopy
NMR nuclear magnetic resonance spectroscopy
PyBOP benzotriazol-1-yloxytris(pyrrolidino)phosphonium hexafluorophosphate
RT room temperature
Rₜ retention time (in HPLC)

### LC/MS, GC/MS and HPLC Methods:

### Method 1:

Instrument: HP 1100 with DAD detection; column: Kromasil RP-18, 60 mm×2 mm, 3.5 μm; eluent A: 5 ml HClO₄/l water, eluent B: acetonitrile; gradient: 0 min 2% B→0.5 min 2% B→4.5 min 90% B→6.5 min 90% B; flow rate: 0.75 ml/min; oven: 30° C.; UV detection: 210 nm.

### Method 2:

Instrument: HP 1100 with DAD detection; column: Kromasil RP-18, 60 mm×2 mm, 3.5 μm; eluent A: 5 ml HClO₄/l water, eluent B: acetonitrile; gradient: 0 min 2% B→0.5 min 2% B→4.5 min 90% B→9 min 90% B; flow rate: 0.75 ml/min; oven: 30° C.; UV detection: 210 nm.

### Method 3:

MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 111 of acetonitrile+0.5 ml of 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 mil/min; oven: 50° C.; UV detection: 210 nm.

### Method 4:

Instrument: Micromass Quattro LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

Method 5:

MS instrument type: Micromass ZQ; HPLC instrument type: HP 1100 series; UV DAD; column: Phenomenex Synergi 2μ Hydro-RP Mercury 20 mm×4 mm; eluent A: 111 of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 mil/min; oven: 50° C.; UV detection: 210 nm.

### Method 6:

Instrument: Micromass GCT, GC 6890; column: Restek RTX-35MS, 30 m×250 μm×0.25 μm; constant flow with helium: 0.88 ml/min; oven: 60° C.; inlet: 250° C.; gradient: 60° C. (hold for 0.30 min), 50° C./min→120° C., 16° C./min ˜250° C., 30° C./min ˜300° C. (hold for 1.7 min).

### Method 7:

Instrument: HP 1100 with DAD detection; column: Kromasil RP-18, 125 mm×4 mm, 5 μm; eluent A: 4 bottles of PIC B7/l of water, eluent B: acetonitrile; PIC B7: heptanesulphonic acid from Millipore/Waters Corp.; gradient: 0.0 min 2% B→1 min 2% B→9 min 90% B→13 min 90% B; flow rate: 2 ml/min; oven: 30° C.; UV detection: 210 nm.

### Starting Compounds and Intermediates:

### Example 1A

### 6-Chloro-2-methyl-4H-3,1-benzoxazin-4-one

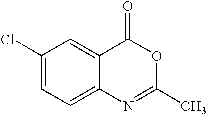

A mixture of 9.42 g of 2-amino-5-chlorobenzoic acid (54.9 mmol) and 31.1 ml of acetic anhydride (33.6 g, 329 mmol) is heated under reflux for 2 h. After cooling, the resulting precipitate is filtered off with suction and washed twice with 50 ml of diethyl ether. 9.01 g (83% of theory) of the product are obtained.

¹H-NMR (400 MHz, DMSO-d₆): δ=3.32 (s, 3H), 7.60 (d, 1H), 7.94 (dd, 1H), 8.06 (d, 1H).

MS (EI): m/z=195 [M]⁺

HPLC (method 1): Rₜ=3.97 min.

### Example 2A

### (2-Amino-5-chlorophenyl)(2,3-dimethoxyphenyl)methanone

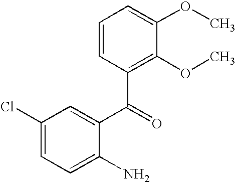

Under argon, 9.07 ml of veratrole (9.28 g, 47.4 mmol) are dissolved in 40 ml of tetrahydrofuran. At 0° C., 22.0 ml of n-butyllithium (3.53 g, 55.0 mmol; 1.6 M solution in hexane) are slowly added. After 30 min., the suspension is added to 9.28 g of the compound from Example 1A in 40 ml of tetrahydrofuran at 0° C. After 30 min., the solvent is removed under reduced pressure. The residue is taken up in 48 ml of ethanol and 20 ml of water, 32 ml of concentrated hydrochloric acid are added, and the mixture is heated under reflux for 3 h. 100 ml of water are added, and the mixture is then extracted three times with 75 ml of diethyl ether each time. The combined organic phases are washed with 1 N sodium hydroxide solution and with saturated sodium chloride solution (100 ml of each), dried over magnesium sulphate and freed of solvent under reduced pressure. The residue is purified by chromatography on a silica gel column (mobile phase: cyclohexane/ethyl acetate 4:1). 6.53 g (47% of theory) of the product are obtained.

¹H-NMR (400 MHz, DMSO-d₆): δ=3.62 (s, 3H), 3.87 (s, 3H), 6.81 (d, 1H), 6.88 (d, 1H), 6.96 (s, 1H), 7.15-7.22 (m, 2H), 7.30 (d, 1H), 7.52 (s, 2H).

MS (CI): m/z=292 [M+H]⁺

HPLC (method 1): Rₜ=4.79 min.

### Example 3A

### [2-(Allylamino)-5-chlorophenyl](2,3-dimethoxyphenyl)methanone

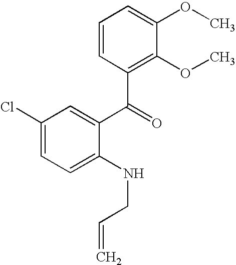

Under argon, 33.2 mg of tetra-n-butylammonium bromide (0.10 mmol) and 1.65 g of sodium hydroxide (41.1 mmol) are added to 3.00 g of the compound from Example 2A (10.3 mmol) in 60 ml of tetrahydrofuran. After stirring at room temperature for 5 min., 2.67 ml of allyl bromide (3.73 g, 30.9 mmol) are added, and the mixture is heated under reflux for two days. Then 150 ml of ethyl acetate are added, and the mixture is extracted with 150 ml of water. The organic phase is separated off, dried over magnesium sulphate and freed of solvent under reduced pressure. The residue is purified by chromatography on a silica gel column (mobile phase: cyclohexane/methylene chloride 1:1). 3.25 g (90% of theory) of the product are obtained.

¹H-NMR (300 MHz, DMSO-d₆): δ=3.63 (s, 3H), 3.88 (s, 3H), 3.96-4.02 (m, 2H), 5.20 (dd, 1H), 5.25 (dd, 1H), 5.91-6.05 (ddt, 1H), 6.83 (dd, 1H), 6.86 (d, 1H), 7.06 (d, 1H), 7.14-7.24 (m, 2H), 7.43 (dd, 1H), 8.95 (t, 1H).

MS (CI): m/z=332 [M+H]⁺

HPLC (method 1): Rₜ=5.36 min.

### Example 4A

### Ethyl (2E)-4-{allyl[4-chloro-2-(2,3-dimethoxybenzoyl)phenyl]amino}-4-oxobut-2-enoate

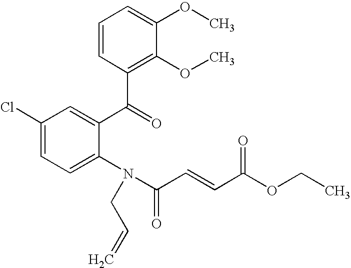

2.92 g of the compound from Example 3A (8.80 mmol) are dissolved in 50 ml of ethyl acetate. To this are added 1.11 g of sodium bicarbonate (13.2 mmol) and 1.57 g of ethyl 3-chlorocarbonylacrylate (9.68 mmol) [preparation analogous to J. Amer. Chem. Soc. 70, 3356-3357 (1948)]. The mixture is stirred at room temperature for 1 h. It is then diluted with 50 ml of ethyl acetate and extracted twice with 75 ml of water. The organic phase is dried over magnesium sulphate and freed of solvent under reduced pressure. The residue is purified by chromatography on a silica gel column (mobile phase: cyclohexane/ethyl acetate 5:1). 3.58 g (89% of theory) of the product are obtained.

MS (CI): m/z=480 [M+H]⁺

HPLC (method 1): Rₜ=5.10 min.

### Example 5A

### Ethyl (2E)-4-(allyl {4-chloro-2-[(2,3-dimethoxyphenyl)(hydroxy)methyl]phenyl}amino)-4-oxobut-2-enoate (racemic)

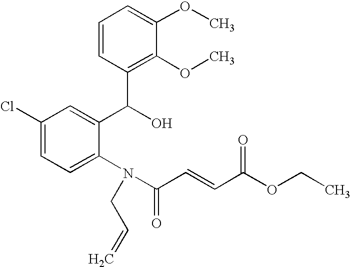

160 mg of sodium borohydride (4.24 mmol) are added to 3.53 g of the compound from Example 4A (7.72 mmol) in 70 ml of ethanol. After stirring at room temperature for 4 h, 150 ml of ethyl acetate are added to the mixture. It is then extracted with 100 ml of saturated sodium chloride solution. The organic phase is dried over magnesium sulphate and freed of solvent under reduced pressure. 3.45 g of the crude product (56% purity, 54% of theory) are obtained and are employed without further purification in the next stage.

LC/MS (method 3): m/z=460.2 [M+H]⁺.

### Example 6A

### Ethyl [1-allyl-7-chloro-5-(2,3-dimethoxyphenyl)-2-oxo-1,2,3,5-tetrahydro-4,1-benzoxazepin-3-yl]-acetate (racemic pair of diastereomers)

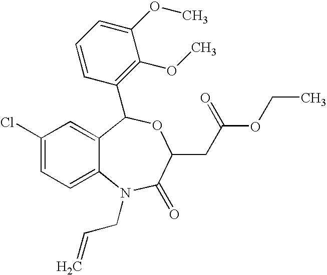

1.04 g of potassium carbonate (7.51 mmol) are added to 3.46 g of the compound from Example 5A (7.51 mmol) in 70 ml of ethanol. After stirring at room temperature overnight, 100 ml of ethyl acetate are added. The mixture is extracted with 100 ml of water and with 100 ml of saturated sodium chloride solution. The organic phase is dried over magnesium sulphate and freed of solvent. The residue is purified by preparative HPLC (eluent: acetonitrile/water with 0.1% formic acid, gradient 10:90→95:5). 2.40 g (69% of theory) of the product are obtained.

MS (ESI): m/z=460.2 [M+H]⁺

HPLC (method 1): Rₜ=5.25 and 5.36 min.

### Example 7A

### Ethyl [7-chloro-5-(2,3-dimethoxyphenyl)-2-oxo-1,2,3,5-tetrahydro-4,1-benzoxazepin-3-yl]acetate (racemic pair of diastereomers)

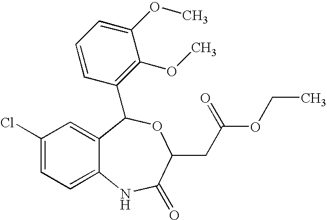

0.2 g of palladium dichloride (1 mmol) is added to 2.38 g of the compound from Example 6A (5.18 mmol) in 20 ml of acetic acid. The mixture is heated under reflux overnight. The mixture is filtered with suction through kieselguhr, and the filtrate is mixed with 100 ml of ethyl acetate. It is extracted three times with 50 ml of saturated sodium chloride solution each time. The organic phase is dried over magnesium sulphate and freed of solvent. The residue is purified by chromatography on a silica gel column (mobile phase: cyclohexane/ethyl acetate 3:1). 1.34 g (56% of theory) of the product are obtained.

MS (CI): m/z=419.9 [M+H]⁺

HPLC (method 1): Rₜ=4.80 and 4.88 min.

### Example 8A

### Ethyl [7-chloro-5-(2,3-dimethoxyphenyl)-2-thioxo-1,2,3,5-tetrahydro-4,1-benzoxazepin-3-yl]acetate (racemic pair of diastereomers)

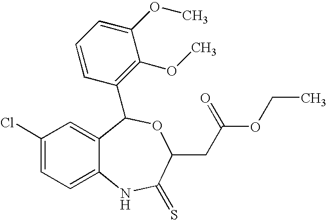

120 mg of sodium bicarbonate (1.43 mmol) and 232 mg of diphosphorus pentasulphide

(1.05 mmol) are added to 400 mg of the compound from Example 7A (0.95 mmol) in 12 ml of 1,2-dimethoxyethane. The mixture is heated under reflux overnight. The mixture is filtered with suction through kieselguhr and the filtrate is concentrated. The residue is purified by chromatography on a silica gel column (mobile phase: cyclohexane/ethyl acetate 4:1). 380 mg (92% of theory) of the product are obtained.

MS (ESI): m/z=436.2 [M+H]⁺

HPLC (method 1): Rₜ=5.14 and 5.19 min.

### Example 9A

### Propanoyl Hydrazide

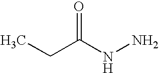

10.0 g of hydrazine hydrate (200 mmol) are heated to boiling. 19.8 ml of ethyl propionate (17.0 g, 166 mmol) are slowly added thereto. The mixture is heated under reflux for 8 h. 7.66 g (44% of theory) of the product are obtained by fractional distillation (120° C./13 mbar).

¹H-NMR (200 MHz, DMSO-d₆): δ=0.99 (t, 3H), 2.01 (q, 2H), 4.10 (br. s, 2H), 8.89 (br. s, 1H).

GC/MS (method 6): Rₜ=3.52 min., m/z=89 [M+H]⁺.

### Example 10A

### Ethyl 4-piperidylacetate

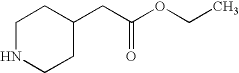

2.0 g of ethyl 4-pyridylacetate in 20 ml of ethanol are mixed with 400 mg of palladium black (20% by weight), adjusted to pH 2 with 1 N hydrochloric acid and hydrogenated at room temperature under 3 bar for 2 days. Solids are filtered off with suction through kieselguhr, and the solvent is removed from the filtrate under reduced pressure. The residue is taken up in 50 ml of ethyl acetate and 50 ml of water. The aqueous phase is adjusted to pH 13 with 1 N sodium hydroxide solution and extracted twice with 50 ml of ethyl acetate each time. The combined organic phases are dried over magnesium sulphate, and the solvent is removed under reduced pressure. 1.21 g (58% of theory) of the product are obtained.

GC/MS (method 6): Rₜ=5.93 min., m/z=172 [M+H]⁺.

### Example 11A

### Ethyl [1-{[(tert-butoxycarbonyl)amino]methyl}-8-chloro-6-(2,3-dimethoxyphenyl)-4H,6H-[1,2,4]-triazolo[4,3-a][4,1]benzoxazepin-4-yl]acetate

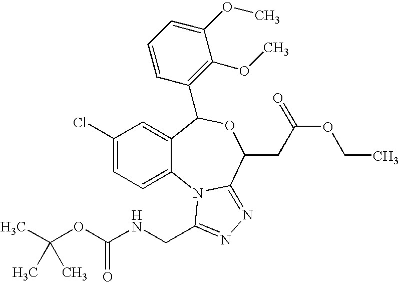

A solution of 590 mg (1.35 mmol) of the compound from Example 8A and 380 mg (2.03 mmol) of N-tert-butoxycarbonylglycine hydrazide [CAS No. 6926-09-6] in 10 ml of dioxane is heated in an autoclave at 140° C. overnight. The solvent is then removed in a rotary evaporator, and the residue is purified by preparative HPLC. 149 mg (19% of theory) of the title compound are obtained.

### Diastereomer Mixture 11A-1:

LC/MS (method 3): Rₜ=2.45 min (59%), m/z=573 [M+H]⁺; 2.52 min (33%), m/z=573 [M+H]⁺.

The diastereomers are separated by chromatography (Reprosol ODS-A, 5 μm, 250 mm×20 mm; eluent: water/acetonitrile (40:60); flow rate: 25 mil/min; oven: 40° C.; UV detection: 210 nm). 63 mg of diastereomer 11A-2 and 41 mg of diastereomer 11A-3 are obtained.

### Diastereomer 11A-2, Racemic:

¹H-NMR (400 MHz, DMSO-d₆): δ=1.18 (t, 3H), 1.27 (s, 9H), 2.83 (broad, 1H), 3.06 (broad, 1H), 3.37 (s, 3H), 3.74 (s, 3H), 4.08 (q, 2H), 4.28-4.33 (m, 1H), 4.44 (broad, 1H), 5.23 (broad, 1H), 6.13 (broad, 1H), 6.58 (broad, 1H), 6.93-6.99 (m, 2H), 7.12 (broad, 1H), 7.33 (broad, 1H), 7.62 (dd, 1H), 7.75 (d, 1H).

### Diastereomer 11A-3, Racemic:

¹H-NMR (300 MHz, DMSO-d₆): δ=1.18 (t, 3H), 1.19 (s, 9H), 3.08 (dd, 1H), 3.28 (dd, 1H), 3.37 (s, 3H), 3.81 (s, 3H), 4.09 (q, 2H), 4.41 (dd, 1H), 4.78 (t, 1H), 4.89 (dd, 1H), 5.56 (s, 1H), 6.61 (d, 1H), 7.11-7.13 (m, 2H), 7.22 (d, 1H), 7.36-7.40 (m, 1H), 7.67 (dd, 1H), 7.89 (d, 1H).

### Example 12A

### Ethyl 4-[(tert-butoxycarbonyl)amino]butanoate

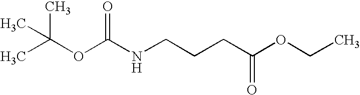

1.0 g (6.0 mmol) of ethyl 4-aminobutyrate hydrochloride is dissolved in 0.9 ml (664 mg, 6.6 mmol) of triethylamine and 10 ml of dichloromethane. The solution is cooled to 0° C., and 1.37 g (6.3 mmol) of di-tert-butyl dicarbonate are added in portions. The mixture is allowed to warm to room temperature and is stirred for 18 hours. 10 ml of 1 N hydrochloric acid are added, and the organic phase is separated off, washed with water and dried over sodium sulphate. The crude product after removal of the solvent in vacuo is purified by preparative HPLC (eluent: acetonitrile/water, gradient 20:80→80:20). 450 mg (33% of theory) of the title compound are obtained.

MS (ESI): m/z=232 [M+H]⁺.

### Example 13A

### tert-Butyl (4-hydrazinyl-4-oxobutyl)carbamate

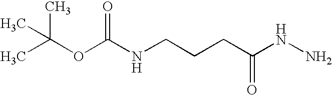

430 mg (1.86 mmol) of the compound from Example 12A are mixed with 103 mg (2.05 mmol) of hydrazine hydrate and 2 ml of ethanol and stirred under reflux for 24 hours. A further 94 mg (1.86 mmol) of hydrazine hydrate are added, and the mixture is stirred under reflux for a further 16 hours. The residue after removal of the solvent is purified by preparative HPLC (eluent: acetonitrile/water, gradient 20:80→80:20). 147 mg (34% of theory) of the title compound are obtained.

HPLC (method 1): Rₜ=3.12 min.

MS (ESI): m/z=218 [M+H]⁺.

### Example 14A

### Ethyl [1-{3-[(tert-butoxycarbonyl)amino]propyl}-8-chloro-6-(2,3-dimethoxyphenyl)-4H,6H-[1,2,4]triazolo[4,3-a][4,1]benzoxazepin-4-yl]acetate (racemic diastereomer)

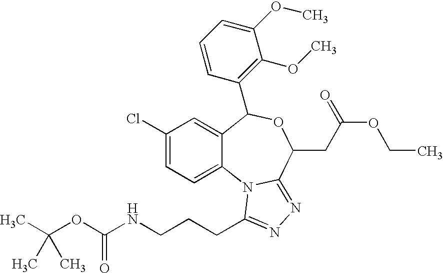

200 mg (0.46 mmol) of the compound from Example 8A and 109 mg (0.50 mmol) of the compound from Example 13A are mixed with 4 ml of dioxane and stirred under reflux for 7 days. The residue after removal of the solvent in vacuo is purified by preparative HPLC (eluent: acetonitrile/water, gradient 20:80→80:20). 40 mg (15% of theory) of the title compound are obtained.

HPLC (method 2): Rₜ=4.96 min.

MS (ESI): m/z=601 [M+H]⁺.

### Example 15A

### Methyl 3-(allyloxy)propanoate

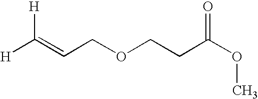

1.0 g (7.68 mmol) of 3-(allyloxy)propionic acid is dissolved in 20 ml of acetone, and 1.59 g (11.5 mmol) of potassium carbonate and 2.4 ml (5.4 g, 38.4 mmol) of iodomethane are added. The mixture is heated under reflux for 5 hours and then the reaction solution is concentrated in vacuo, and the residue is taken up in 20 ml of a 10% strength aqueous potassium carbonate solution. 20 ml of diethyl ether are added, and the organic phase is separated off and washed with water and a saturated aqueous sodium chloride solution. The organic extracts are dried over sodium sulphate, filtered and concentrated in vacuo. 877 mg (79% of theory) of the title compound are obtained.

GC/MS (method 6): Rₜ=3.54 min., m/z=113 [M-OCH₃]⁺.

### Example 16A

### 3-(Allyloxy)propanohydrazide

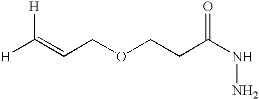

860 mg (6.0 mmol) of the compound from Example 15A are dissolved in 10 ml of methanol.

896 mg (17.9 mmol) of hydrazine hydrate are added, and the mixture is stirred at room temperature overnight. The solution is concentrated, and residues of hydrazine are removed in vacuo. 835 mg (90% of theory) of the title compound are isolated.

HPLC (method 7): R=9.68 min.

MS (DCI): m/z=145 [M+H]⁺.

### Example 17A

### (2-Amino-5-chlorophenyl)(2-methoxyphenyl)methanone

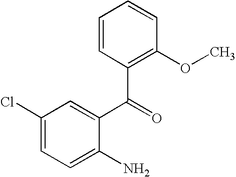

3.00 g (15.3 mmol) of the compound from Example 1A are dissolved in 10 ml of tetrahydrofuran and cooled to 0° C. At this temperature, 18.4 ml of a 1 N solution of 2-methoxyphenylmagnesium bromide in tetrahydrofuran are added dropwise. The mixture is stirred at room temperature for 1 hour and, after addition of 10 ml of 1 N hydrochloric acid, extracted with 25 ml of ethyl acetate, and the organic phase is concentrated. The residue is taken up in 20 ml of ethanol and 10 ml of 50% concentrated hydrochloric acid and heated under reflux for 4 hours. A pH of 9 is adjusted with 1 N aqueous sodium hydroxide solution, and the mixture is extracted with 25 ml of ethyl acetate. The organic phase is washed with saturated aqueous sodium chloride solution, dried over sodium sulphate and concentrated in vacuo. The residue is purified by column chromatography on silica gel (eluent: cyclohexane/ethyl acetate 9:1). 1.22 g (30% of theory) of the title compound are obtained.

LC/MS (method 3): Rₜ=2.34 min., m/z=262 [M+H]⁺.

### Example 18A

### [2-(Allylamino)-5-chlorophenyl](2-methoxyphenyl)methanone

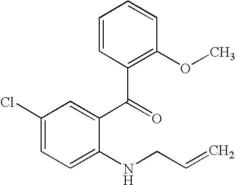

1.53 g (5.85 mmol) of the compound from Example 17A are dissolved in 35 ml of tetrahydrofuran, and 935 mg (23.38 mmol) of sodium hydroxide and 19 mg (0.06 mmol) of tetra-n-butylammonium bromide are added. 1.52 ml (2.12 g, 17.54 mmol) of allyl bromide are added, and the reaction mixture is stirred at 60° C. for 16 hours. The residue after removal of the solvent in vacuo is mixed with ethyl acetate, washed with water, dried over sodium sulphate and purified by column chromatography on silica gel (eluent: cyclohexane/ethyl acetate 10:1). 1.02 g (56% of theory) of the title compound are obtained.

HPLC (method 1): Rₜ=5.32 min.

MS (ESI): m/z=302 [M+H]⁺.

### Example 19A

### Ethyl (2E)-4-{allyl[4-chloro-2-(2-methoxybenzoyl)phenyl]amino}-4-oxobut-2-enoate

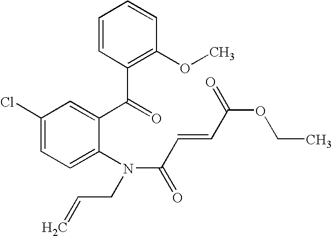

1.0 g (3.3 mmol) of the compound from Example 18A is introduced into 10 ml of ethyl acetate, and 418 mg (5.0 mmol) of sodium bicarbonate are added. While cooling in ice, a solution of 646 mg (4.0 mmol) of ethyl (2E)-4-chloro-4-oxobut-2-enoate in 10 ml of ethyl acetate is added. The reaction solution is stirred at room temperature overnight and then diluted with 20 ml of ethyl acetate. The organic phase is washed with water and saturated aqueous sodium chloride solution, dried over sodium sulphate and concentrated in vacuo, and the residue is purified by column chromatography on silica gel (eluent: cyclohexane/ethyl acetate 4:1). 1.15 g (81% of theory) of the title compound are obtained.

HPLC (method 1): Rₜ=5.06 min.

MS (ESI): m/z=428 [M+H]⁺.

### Example 20A

### Ethyl [1-allyl-7-chloro-5-(2-methoxyphenyl)-2-oxo-1,2,3,5-tetrahydro-4,1-benzoxazepin-3-yl]acetate (racemic mixture of diastereomers)

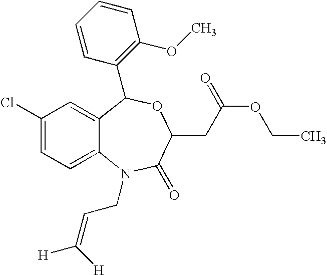

1.10 g (2.6 mmol) of the compound from Example 19A are dissolved in 20 ml of ethanol. 53 mg (1.4 mmol) of sodium borohydride are added, and the reaction mixture is stirred at room temperature for 1 day. 10 ml of 1 N hydrochloric acid are added, and the mixture is extracted with ethyl acetate. The organic extracts are washed with water and a saturated aqueous sodium chloride solution. The residue after removal of the solvent in vacuo is purified by column chromatography on silica gel (eluent: cyclohexane/ethyl acetate 4:1). 736 mg (67% of theory) of the title compound are obtained.

HPLC (method 2): Rₜ=5.24 min. (diastereomer 1) and 5.35 min. (diastereomer 2)

MS (ESI): m/z=430 [M+H]⁺.

### Example 21A

### Ethyl [7-chloro-5-(2-methoxyphenyl)-2-oxo-1,2,3,5-tetrahydro-4,1-benzoxazepin-3-yl]acetate (racemic mixture of diastereomers)

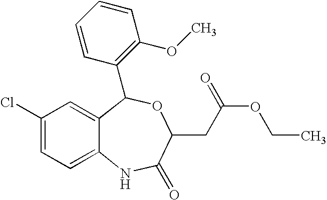

670 mg (1.6 mmol) of the compound from Example 20A are introduced into 6 ml of acetic acid. 55 mg (0.3 mmol) of palladium(II) chloride are added, and the reaction mixture is stirred under reflux for 36 hours. The reaction mixture is filtered through kieselguhr, washed with ethyl acetate and concentrated in vacuo. The residue is taken up in ethyl acetate and washed with a saturated aqueous sodium bicarbonate solution and a saturated aqueous sodium chloride solution. The residue after removal of the solvent in vacuo is purified by preparative HPLC (eluent: acetonitrile/water, gradient 20:80→80:20). 150 mg (25% of theory) of the title compound are obtained.

HPLC (method 2): Rₜ=4.79 min. (diastereomer 1) and 4.86 min. (diastereomer 2)

MS (ESI): m/z=390 [M+H]⁺.

### Example 22A

### Ethyl [7-chloro-5-(2-methoxyphenyl)-2-thioxo-1,2,3,5-tetrahydro-4,1-benzoxazepin-3-yl]acetate (racemic mixture of diastereomers)

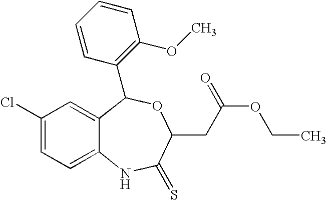

80 mg (0.4 mmol) of diphosphorus pentasulphide and 40 mg (0.5 mmol) of sodium bicarbonate are added to 120 mg (0.3 mmol) of the compound from Example 21A. 4 ml of 1,2-dimethoxyethane are added, and the reaction mixture is heated to reflux for 1 hour. It is subsequently filtered through kieselguhr, the filtrate is concentrated in vacuo, and the residue is mixed with ethyl acetate. The organic phase is washed with a saturated aqueous sodium bicarbonate solution and a saturated aqueous sodium chloride solution. The residue after removal of the solvent in vacuo is purified by column chromatography on silica gel (eluent: cyclohexane/ethyl acetate 7:3). 105 mg (82% of theory) of the title compound are obtained.

LC/MS (method 4): Rₜ=2.82 min. (diastereomer 1) and 2.87 min. (diastereomer 2), m/z=406 [M+H]⁺.

### Example 23A

### N-Allyl-4-chloroaniline

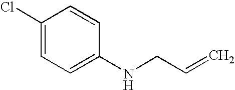

4.0 g (31.4 mmol) of 4-chloroaniline are dissolved in 80 ml of tetrahydrofuran, and 5.0 g (125.4 mmol) of sodium hydroxide and 101 mg (0.3 mmol) of tetra-n-butylammonium bromide are added. 4.1 ml (5.7 g, 47.0 mmol) of allyl bromide are added, and the reaction mixture is stirred at 60° C. for 16 hours. The residue after removal of the solvent in vacuo is mixed with ethyl acetate, washed with water, dried over sodium sulphate and purified by column chromatography on silica gel (eluent: cyclohexane/ethyl acetate 9:1). 2.0 g (39% of theory) of the title compound are obtained.

HPLC (method 1): Rₜ=3.62 min.

MS (ESI): m/z=168 [M+H]⁺.

### Example 24A

### [2-(Allylamino)-5-chlorophenyl](2,3-dihydro-1,4-benzodioxin-5-yl)methanol

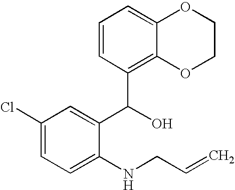

900 mg (5.5 mmol) of boron trichloride are introduced into 6 ml of toluene. A solution of 920 mg (5.5 mmol) of the compound from Example 23A in 2 ml of toluene is added. The mixture is stirred at 90° C. for 4 hours. Then, while cooling in an ice bath, a solution of 901 mg (5.5 mmol) of 2,3-dihydro-1,4-benzodioxin-5-carbaldehyde and 0.92 ml (666 mg, 6.6 mmol) of triethylamine in 3 ml of toluene is added. The mixture is stirred at 0° C. for 30 minutes and at room temperature for 16 hours. Water is then added, and the solution is made basic by adding sodium bicarbonate. It is extracted with ethyl acetate, the organic extracts are dried over sodium sulphate, the solvent is removed in vacuo, and the residue is purified by column chromatography on silica gel (eluent: cyclohexane/ethyl acetate 9:1). 450 mg (24% of theory) of the title compound are obtained.

HPLC (method 2): Rₜ=4.50 min.

MS (DCI): m/z=332 [M+H]⁺.

### Example 25A

### Ethyl (2E)-4-(allyl {4-chloro-2-[2,3-dihydro-1,4-benzodioxin-5-yl(hydroxy)methyl]phenyl}amino)-4-oxobut-2-enoate

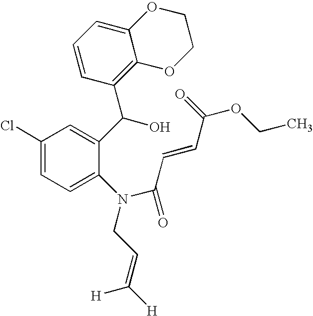

400 mg (1.2 mmol) of the compound from Example 24A are introduced into 6 ml of ethyl acetate, and 180 mg (1.3 mmol) of potassium carbonate are added. While cooling in ice, a solution of 646 mg (4.0 mmol) of ethyl (2E)-4-chloro-4-oxobut-2-enoate in 6 ml of ethyl acetate is added. The reaction solution is stirred at room temperature overnight and then diluted with 20 ml of ethyl acetate. The organic phase is washed with water and saturated aqueous sodium chloride solution, dried over sodium sulphate and concentrated in vacuo. The crude product is reacted directly without further purification in the next stage.

### Example 26A

### Ethyl [1-allyl-7-chloro-5-(2,3-dihydro-1,4-benzodioxin-5-yl)-2-oxo-1,2,3,5-tetrahydro-4,1-benzoxazepin-3-yl]acetate (racemic mixture of diastereomers)

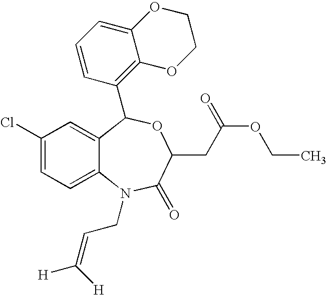

220 mg (0.48 mmol) of the compound from Example 25A are dissolved in 4 ml of ethanol. 70 mg (0.48 mmol) of potassium carbonate are added and the reaction mixture is stirred at room temperature for 16 hours. Water is added, and the solid is filtered off. 165 mg (75% of theory) of the title compound are obtained.

HPLC (method 2): Rₜ=4.70 min. (diastereomer 1) and 4.85 min. (diastereomer 2)

MS (ESI): m/z=458 [M+H]⁺.

### Example 27A

### Ethyl [7-chloro-5-(2,3-dihydro-1,4-benzodioxin-5-yl)-2-oxo-1,2,3,5-tetrahydro-4,1-benzoxazepin-3-yl]acetate (racemic mixture of diastereomers)

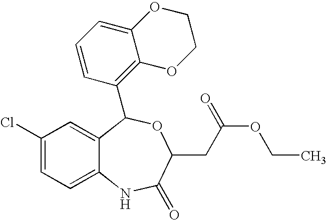

150 mg (0.33 mmol) of the compound from Example 26A are dissolved in 3 ml of toluene. 7 mg (0.01 mmol) of 1,3-bis(diphenylphosphino)propanenickel(II) chloride and, over a period of 5 minutes, 0.18 ml (26 mg, 0.36 mmol) of a 2 M solution of trimethylaluminium in toluene are added. Further portions of 0.03 ml (5 mg, 0.07 mmol) of the 2 M trimethylaluminium solution in toluene are added after 90 minutes, 210 minutes and 240 minutes. The reaction solution is then stirred into 6 ml of a 1:1 mixture of ethyl acetate and a 50% concentrated potassium sodium tartrate solution. The organic phase is separated off and dried over sodium sulphate, the solvent is removed in vacuo, and the residue is purified by column chromatography on silica gel (eluent: cyclohexane/ethyl acetate 7:3). 70 mg (50% of theory) of the title compound are obtained.

HPLC (method 1): Rₜ=4.81 min.

MS (ESI): m/z=418 [M+H]⁺.

### Example 28A

### Ethyl [7-chloro-5-(2,3-dihydro-1,4-benzodioxin-5-yl)-2-thioxo-1,2,3,5-tetrahydro-4,1-benzoxazepin-3-yl]acetate (racemic mixture of diastereomers)

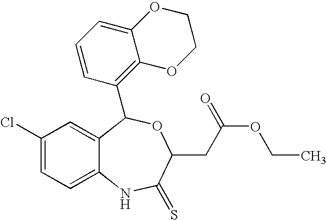

38 mg (0.17 mmol) of diphosphorus pentasulphide and 20 mg (0.23 mmol) of sodium bicarbonate are added to 65 mg (0.16 mmol) of the compound from Example 27A. 2 ml of 1,2-dimethoxyethane are added, and the reaction mixture is heated to reflux for 3 hours. It is then filtered through kieselguhr, the filtrate is concentrated in vacuo, and the residue is mixed with ethyl acetate. The organic phase is washed with a saturated aqueous sodium bicarbonate solution and a saturated aqueous sodium chloride solution. The residue after removal of the solvent in vacuo is purified by column chromatography on silica gel (eluent: cyclohexane/ethyl acetate 4:1). 57 mg (84% of theory) of the title compound are obtained.

LC/MS (method 3): Rₜ=2.60 min., m/z=434 [M+H]⁺.

### Example 29A

### tert-Butyl (3-hydrazino-2,2-dimethyl-3-oxopropyl)carbamate

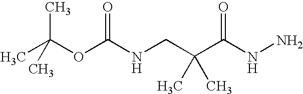

2.20 g (8.97 mmol) of ethyl 3-[(tert-butoxycarbonyl)amino]-2,2-dimethylpropanoate are stirred in 2.18 ml (44.8 mmol) of hydrazine hydrate under reflux for 2 days. The mixture is purified directly by preparative HPLC (eluent: acetonitrile/water, gradient 10:90→95:5). 1.09 g (53% of theory) of the title compound are obtained.

¹H-NMR (400 MHz, CDCl₃): δ=1.19 (s, 6H), 1.44 (s, 9H), 3.24 (d, 2H), 3.88 (br, 2H), 5.11 (br, 1H), 7.44 (br, 1H).

LC/MS (method 5): Rₜ=1.26 min., m/z=232 [M+H]⁺.

### Example 30A

### [1-{2-[(tert-Butoxycarbonyl)amino]-1,1-dimethylethyl}-8-chloro-6-(2,3-dimethoxyphenyl)-4H,6H-[1,2,4]triazolo[4,3-a][4,1]benzoxazepin-4-yl]acetate ethyl ester

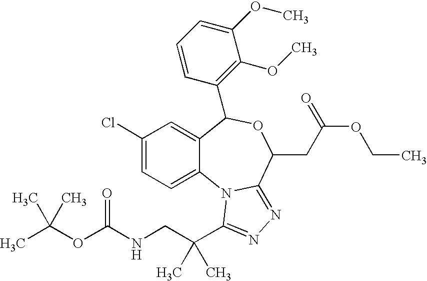

500 mg (1.15 mmol) of the compound from Example 8A are stirred with 424 mg (1.84 mmol) of the compound from Example 29A in 5 ml of dioxane under reflux for 3 days. The solvent is removed under reduced pressure, and the residue is purified by preparative HPLC (eluent: acetonitrile/water, gradient 10:90→95:5). 139 mg (19.7% of theory) of the racemic diastereomer 30A-1 and 134 mg (19.0% of theory) of the racemic diastereomer 30A-2 are obtained.

### Diastereomer 30A-1, Racemic:

HPLC (method 2): Rₜ=5.04 min.

### Diastereomer 30A-2, Racemic:

¹H-NMR (400 MHz, DMSO-d₆): δ=1.18 (s, 3H and t, 3H), 1.31 (s, 12H), 3.08 (dd, 1H), 3.18-3.26 (m, 2H), 3.38 (s, 3H), 3.57 (dd, 1H), 3.81 (s, 3H), 4.08 (q, 2H), 4.56 (t, 1H), 5.36 (s, 1H), 6.57 (d, 1H), 7.03-7.14 (m, 3H), 7.21 (t, 1H), 7.77 (dd, 1H), 8.12 (d, 1H).

HPLC (method 2): Rₜ=5.22 min.

MS (ESI): m/z=615.5 [M+H]⁺.

### EXEMPLARY EMBODIMENTS

### Example 1

### Ethyl [8-chloro-6-(2,3-dimethoxyphenyl)-1-methyl-4H,6H-[1,2,4]triazolo[4,3-a][4,1]benzoxazepin-4-yl]acetate

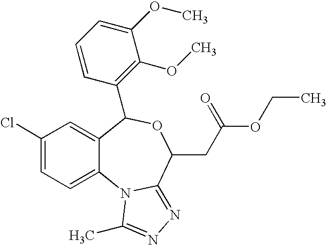

158 mg of acetohydrazide (2.06 mmol) are added to 500 mg of the compound from Example 8A (1.15 mmol) in 23 ml of dioxane. The mixture is heated under reflux for 4 days. The solvent is removed under reduced pressure, and the residue is purified by preparative HPLC (eluent: acetonitrile/water with 0.1% formic acid, gradient 10:90→95:5). 261 mg (49% of theory) of the title compound are obtained.

MS (ESI): m/z=458.3 [M+H]⁺

HPLC (method 1): Rₜ=4.53 and 4.63 min.

The diastereomers and enantiomers are separated by preparative HPLC on a chiral phase [Agilent 1100 with DAD detection; column: Daicel Chiralpak AD-H, 5 μm, 250 mm×20 mm; eluent: isohexane/ethanol 1:1; flow rate: 15 ml/min.; oven: 30° C.; UV detection: 220 nm]:

### Stereoisomer 1-1:

Rₜ=6.87 min. [column: Daicel Chiralpak AD-H, 5 μm, 250 mm×4.6 mm; eluent: isohexane/ethanol 1:1; flow rate: 1 ml/min.; oven: 30° C.; UV detection: 220 nm n]

### Stereoisomer 1-2:

Rₜ=7.41 min.

### Stereoisomer 1-3:

Rₜ=10.11 min.

### Stereoisomer 14:

Rₜ=10.76 min.

¹H-NMR (400 MHz, DMSO-d₆): δ=1.18 (t, 3H), 3.08 and 3.27 (AB signal, additionally split as d, 2H), 3.35 (s, 3H), 3.73 (s, 3H), 4.09 (q, 2H), 4.80 (dd, 1H), 5.45 (s, 1H), 6.68 (s, 1H), 7.13 (dd, 1H), 7.75 (dd, 1H), 7.88 (d, 1H).

### Example 2

### Ethyl [8-chloro-6-(2,3-dimethoxyphenyl)-1-isopropyl-4H,6H-[1,2,4]triazolo[4,3-a][4,1]benzoxazepin-4-yl]acetate (racemic pair of diastereomers)

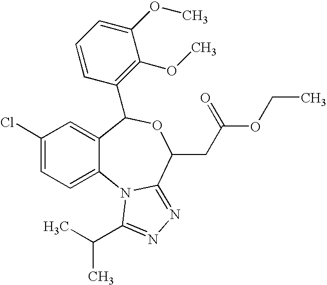

The title compound is prepared in analogy to Example 1 (the appropriate starting acyl hydrazide is prepared in analogy to Example 9A).

HPLC (method 2): Rₜ=4.60 and 4.73 min.

MS (ESI): nm/z=486.3 [M+H]⁺.

The diastereomers and enantiomers are separated by preparative HPLC on a chiral phase [Agilent 1100 with DAD detection; column: Daicel Chiralpak AD-H, 5 μm, 250 mm×20 mm; eluent: isohexane/isopropanol 1:1 or isohexane/ethanol 1:1; flow rate: 15 ml/min.; oven: 25° C.; UV detection: 220 nm]:

### Stereoisomer 2-1:

Rₜ=4.38 min. [column: Daicel Chiralpak AD-H, 5 μm, 250 mm×4.6 mm; eluent: isohexane/isopropanol 1:1; flow rate: 1 ml/min.; oven: 25° C.; UV detection: 220 nm]

### Stereoisomer 2-2:

Rₜ=5.48 min.

¹H-NMR (400 MHz, DMSO-d₆): δ=0.99 (d, 3H), 1.17 (t, 3H), 1.52 (d, 3H), 3.08 and 3.25 (AB signal, additionally split as d, 2H), 3.32 (s, 3H), 3.51 (septet, 1H), 3.80 (s, 3H), 4.08 (q, 2H), 4.76 (dd, 1H), 5.35 (s, 1H), 6.62 (s, 1H), 7.12-7.17 (m, 2H), 7.22 (dd, 1H), 7.73 (dd, 1H), 7.95 (d, 1H).

### Stereoisomer 2-3:

R=5.55 min.

### Stereoisomer 2-4:

Rₜ=7.11 min.

### Example 3

### [8-Chloro-6-(2,3-dimethoxyphenyl)-1-isopropyl-4H,6H-[1,2,4]triazolo[4,3-a][4,1]benzoxazepin-4-yl]acetic acid (racemic diastereomer)

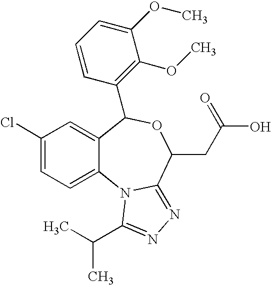

1.27 g (2.61 mmol) of the compound from Example 2 (racemic pair of diastereomers) in 15 ml of ethanol are mixed with 1 ml of concentrated hydrochloric acid and stirred at 80° C. for 2 days. The solvent is removed under reduced pressure, and the residue is purified by preparative HPLC (eluent: acetonitrile/water with 0.1% formic acid, gradient 10:90→95:5). 814 mg (68% of theory) of the product are obtained.

¹H-NMR (400 MHz, DMSO-d₆): δ=0.99 and 1.51 (2 d, 6H), 3.00 and 3.23 (AB signal, additionally split as d, 2H), 3.34 (s, 3H), 3.50 (septet, 1H), 3.82 (s, 3H), 4.72 (dd, 1H), 5.34 (s, 1H), 6.63 (d, 1H), 7.12-7.18 (m, 2H), 7.23 (dd, 1H), 7.74 (dd, 1H), 7.96 (d, 1H), 12.5 (br. s, 1H).

LC/MS (method 3): Rₜ=2.08 min., m/z=458 [M+H]⁺.

### Stereoisomer 3-3:

Starting from stereoisomer 2-3 from Example 2, the corresponding diastereomerically and enantiomerically pure compound is obtained in an analogous manner in a yield of 74% of theory.

¹H-NMR (400 MHz, DMSO-d₆): δ=0.98 (d, 3H), 1.55 (d, 3H), 3.01 and 3.23 (AB signal, additionally split as d, 2H), 3.32 (s, 3H), 3.51 (septet, 1H), 3.82 (s, 3H), 4.72 (dd, 1H), 5.36 (s, 1H), 6.63 (d, 1H), 7.12-7.19 (m, 2H), 7.25 (dd, 1H), 7.75 (dd, 1H), 7.96 (d, 1H), 12.48 (br. s, 1H).

LC/MS (method 3): Rₜ=2.07 min., m/z=458 [M+H]⁺

[α]=+6.2° (solvent: dichloromethane, wavelength: 589 nm, temperature: 19.9° C., concentration 0.5150 g/100 ml, path length 100.0 mm).

### Example 4

### 8-Chloro-6-(2,3-dimethoxyphenyl)-1-isopropyl-4-(2-oxo-2-piperidin-1-ylethyl)-4H,6H-[1,2,4]-triazolo[4,3-a][4,1]benzoxazepine (racemic diastereomer)

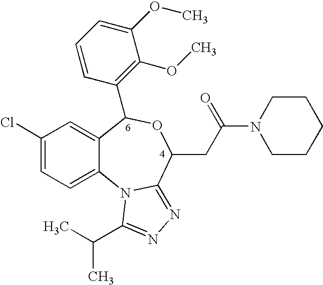

58.9 mg of PyBOP (0.113 mmol) and 14.6 mg of N,N-diisopropylethylamine (0.113 mmol) are added to 47.1 mg (0.103 mmol) of the compound from Example 3 in 2 ml of tetrahydrofuran and 100 μl of dimethylformamide. After stirring at room temperature for 1 h, 11.1 μl of piperidine (9.63 mg, 0.113 mmol) are added. After 1 h, the solvent is removed under reduced pressure. The residue is purified by preparative HPLC (eluent: acetonitrile/water with 0.1% formic acid, gradient 10:90→95:5). 49.3 mg (91% of theory) of the title compound are obtained.

¹H-NMR (400 MHz, DMSO-d₆): δ=0.99 and 1.51 (2 d, 6H), 1.36-1.44 (m, 2H), 1.48-1.62 (m, 4H), 3.05 (dd, 1H), 3.33 (s, 3H), 3.34-3.45 (m, 1H), 3.81 (s, 3H), 4.80 (dd, 1H), 5.31 (s, 1H), 6.63 (d, 1H), 7.13 (dd, 1H), 7.17-7.26 (m, 2H), 7.74 (dd, 1H), 7.96 (d, 1H).

HPLC (method 2): Rₜ=4.78 min.

MS (ESI): m/z=525.3 [M+H]⁺.

The enantiomers are separated by preparative HPLC on a chiral phase [Agilent 1100 with DAD detection; column: Daicel Chiralpak AD-H, 5 μm, 250 mm×20 mm; eluent: methanol; flow rate: 18 ml/min.; oven: 24° C.; UV detection: 260 nm]:

### Enantiomer 4-1:

Rₜ=4.26 min. [column: Daicel Chiralpak AD-H, 5 μm, 250 mm×4.6 mm; eluent: methanol; flow rate: 1 ml/min.; oven: 24° C.; UV detection: 260 nm]

### Enantiomer 4-2:

Rₜ=11.41 min.

LC/MS (method 3): Rₜ=2.47 min., m/z=525 [M+H]⁺

[α]=−28.3° (solvent: dichloromethane, wavelength: 589 nm, temperature: 19.9° C., concentration 0.500 g/100 ml, path length 100.0 mm).

X-Ray structure analysis of enantiomer 4-2 revealed an S configuration at C-6 and an R configuration at C-4 for this stereoisomer.

### Example 5

### Ethyl [8-chloro-6-(2,3-dimethoxyphenyl)-1-ethyl-4H,6H-[1,2,4]triazolo[4,3-a][4,1]benzoxazepin-4-yl]acetate (racemic pair of diastereomers)

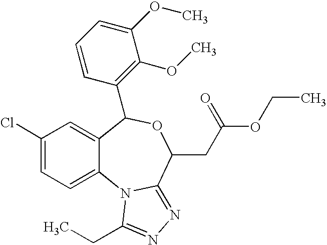

The title compound is prepared in analogy to Example 1.

MS (ESI): m/z=472.2 [M+H]⁺

HPLC (method 2): Rₜ=4.71 and 4.87 min.

### Example 6

### Ethyl [1-sec-butyl-8-chloro-6-(2,3-dimethoxyphenyl)-4H,6H-[1,2,4]triazolo[4,3-a][4,1]benzoxazepin-4-yl]acetate (racemic pair of diastereomers)

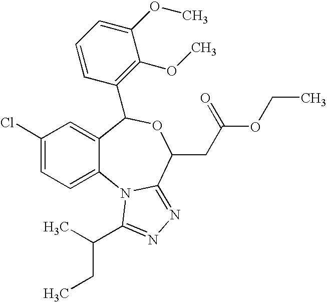

The title compound is prepared in analogy to Example 1 (the appropriate starting acyl hydrazide is prepared in analogy to Example 9A).

LC/MS (method 4): Rₜ=2.78 and 2.81 min., m/z=500.2 [M+H]⁺.

### Example 7

### Ethyl [8-chloro-1-cyclohexyl-6-(2,3-dimethoxyphenyl)-4H,6H-[1,2,4]triazolo[4,3-a][4,1]benzoxazepin-4-yl]acetate (racemic diastereomer)

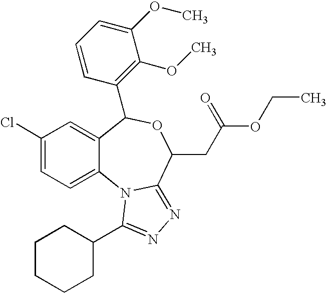

The title compound is prepared in analogy to Example 1 (the appropriate starting acyl hydrazide is prepared in analogy to Example 9A).

HPLC (method 2): Rₜ=5.28 min.

MS (ESI): m/z=526.3 [M+H]⁺.

### Example 8

### Ethyl [8-chloro-1-cyclopropyl-6-(2,3-dimethoxyphenyl)-4H,6H-[1,2,4]triazolo[4,3-a][4,1]benzoxazepin-4-yl]acetate (racemic diastereomer)

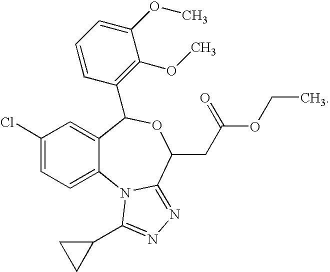

The title compound is prepared in analogy to Example 1 (the appropriate starting acyl hydrazide is prepared in analogy to Example 9A).

HPLC (method 2): Rₜ=4.77 min.

MS (ESI): m/z=484.2 [M+H]⁺.

The following compound is prepared in analogy to the examples described above from the appropriate starting compounds:

### Example 9

### Ethyl [8-chloro-6-(2,3-dimethoxyphenyl)-1-(1-ethylpropyl)-4H,6H-[1,2,4]triazolo[4,3-a][4,1]benzoxazepin-4-yl]acetate

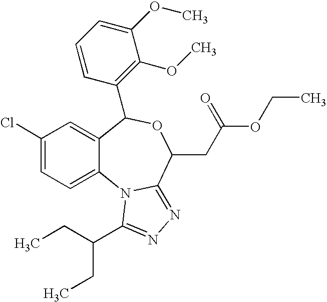

### Example 10

### Ethyl [8-chloro-6-(2,3-dimethoxyphenyl)-1-isobutyl-4H,6H-[1,2,4]triazolo[4,3-a][4,1]benzoxazepin-4-yl]acetate

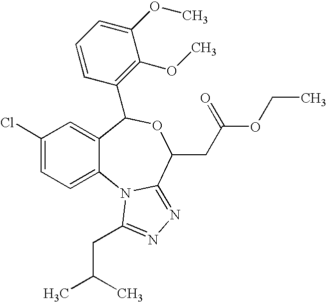

62.5 mg of 3-methylbutanoyl hydrazide (0.57 mmol) are added to 50 mg of the compound from Example 8A (0.11 mmol) in 23 ml of dioxane. The mixture is heated under reflux for 6 days. A further 30 mg of 3-methylbutanoyl hydrazide (0.26 mmol) are added, and the mixture is heated under reflux overnight. The solvent is removed under reduced pressure, and the residue is purified by preparative HPLC (eluent: acetonitrile/water with 0.1% formic acid, gradient 10:90→95:5). 11 mg (18% of theory) of the racemic diastereomer 10-1 and 22 mg (39% of theory) of the racemic diastereomer 10-2 are obtained.

### Diastereomer 10-1 Racemic:

¹H-NMR (300 MHz, DMSO-d₆): δ=0.82 (d, 3H), 0.86 (d, 3H), 1.19 (t, 3H), 1.83 (septet, 1H), 2.90 and 3.02 (AB signal, additionally split to doublet, 2H), 3.09 and 3.29 (AB signal, additionally split to doublet, 2H), 3.35 (s, 3H), 3.82 (s, 3H), 4.11 (q, 2H), 4.80 (dd, 1H), 5.49 (s, 1H), 6.63 (d, 1H), 7.12-7.17 (m, 2H), 7.20-7.27 (m, 1H), 7.75 (dd, 1H), 7.90 (d, 1H).

LC/MS (method 5): Rₜ=2.84 min., m/z=500.1 [M+H]⁺.

### Diastereomer 10-2, Racemic:

LC/MS (method 5): Rₜ=2.71 min., m/z=500.1 [M+H]⁺.

The following compounds are prepared in analogy to the examples described above from the appropriate starting compounds:

### Example 11

### Ethyl [8-chloro-6-(2-fluorophenyl)-1-methyl-4H,6H-[1,2,4]triazolo[4,3-a][4,1]benzoxazepin-4-yl]-acetate

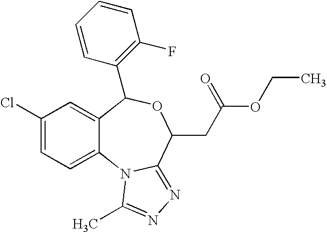

### Example 12

### Ethyl [8-chloro-1-ethyl-6-(2-fluorophenyl)-4H,6H-[1,2,4]triazolo[4,3-a][4,1]benzoxazepin-4-yl]-acetate

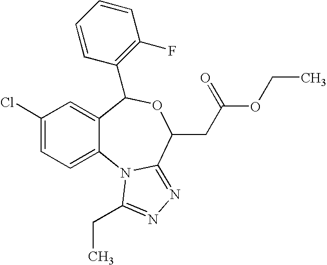

### Example 13

### Ethyl [8-chloro-6-(2-fluorophenyl)-1-isopropyl-4H,6H-[1,2,4]triazolo[4,3-a][4,1]benzoxazepin-4-yl]acetate

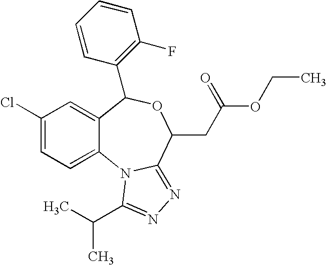

### Example 14

### tert-Butyl [8-chloro-6-(2,3-dimethoxyphenyl)-1-isopropyl-4H,6H-[1,2,4]triazolo[4,3-a][4,1]benzoxazepin-4-yl]acetate

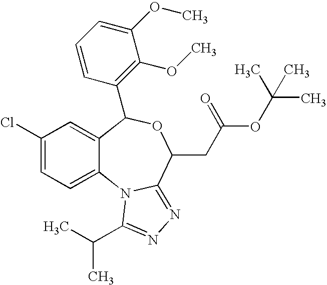

### Example 15

### 2-[8-Chloro-6-(2,3-dimethoxyphenyl)-1-isopropyl-4H,6H-[1,2,4]triazolo[4,3-a][4,1]benzoxazepin-4-yl]-N-ethylacetamide

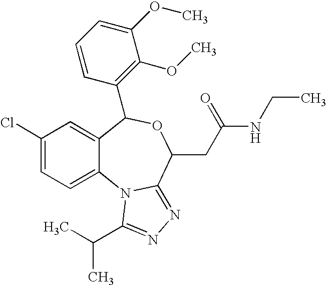

### Example 16

### 2-[8-Chloro-6-(2,3-dimethoxyphenyl)-1-isopropyl-4H,6H-[1,2,4]triazolo[4,3-a][4,1]benzoxazepin-4-yl]-N-ethyl-N-methylacetamide

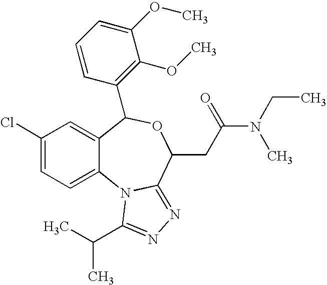

### Example 17

### Methyl 4-({[8-chloro-6-(2,3-dimethoxyphenyl)-1-isopropyl-4H,6H-[1,2,4]triazolo[4,3-a][4,1]-benzoxazepin-4-yl]acetyl}amino)butanoate

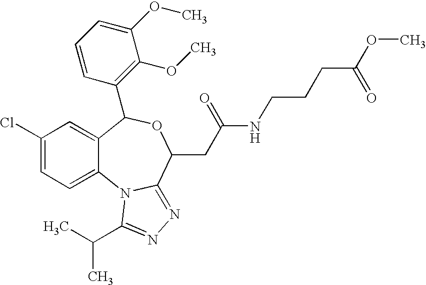

### Example 18

### 4-({[8-Chloro-6-(2,3-dimethoxyphenyl)-1-isopropyl-4H,6H-[1,2,4]triazolo[4,3-a][4,1]benzoxazepin-4-yl]acetyl}amino)butanoic acid

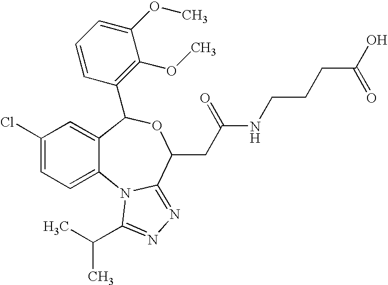

### Example 19

### N-{[8-Chloro-6-(2,3-dimethoxyphenyl)-1-isopropyl-4H,6H-[1,2,4]triazolo[4,3-a][4,1]-benzoxazepin-4-yl]acetyl}-beta-alanine methyl ester

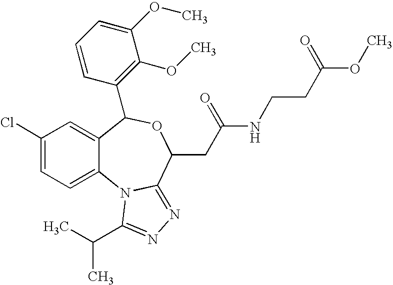

### Example 20

### N-{[8-Chloro-6-(2,3-dimethoxyphenyl)-1-isopropyl-4H,6H-[1,2,4]triazolo[4,3-a][4,1]benzoxazepin-4-yl]acetyl}-beta-alanine

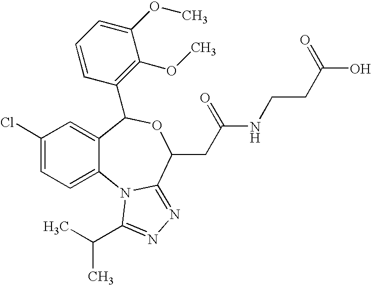

### Example 21

### 8-Chloro-6-(2,3-dimethoxyphenyl)-1-isopropyl-4-(2-oxo-2-pyrrolidin-1-ylethyl)-4H,6H-[1,2,4]-triazolo[4,3-a][4,1]benzoxazepine

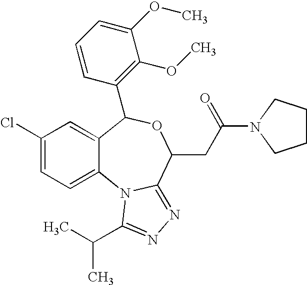

62.5 mg of PyBOP (0.120 mmol) and 21 μl of N,N-diisopropylethylamine (15.5 mg, 0.120 mmol) are added to 50 mg of the compound from Example 3 (0.109 mmol) in 2 ml of tetrahydrofuran and 50 μl of dimethylformamide at 0° C. The mixture is stirred at RT for 1 h and then 10 μl of pyrrolidine (8.5 mg, 0.12 mmol) are added. The mixture is stirred at RT for 1 h and then the solvent is removed under reduced pressure. The residue is purified by preparative HPLC (eluent: acetonitrile/water with 0.1% formic acid, gradient 10:90→95:5). 22 mg (40% of theory) of the title compound are obtained.

¹H-NMR (300 MHz, DMSO-d₆): δ=1.00 (d, 3H), 1.52 (d, 3H), 1.72-1.96 (m, 4H), 3.00 (dd, 1H), 3.22-3.31 (m, 3H), 3.35 (s, 3H), 3.46-3.64 (m, 3H), 3.82 (s, 3H), 4.80 (dd, 1H), 5.36 (s, 1H), 6.63 (d, 1H), 7.11-7.28 (m, 3H), 7.74 (dd, 1H), 7.96 (d, 1H).

LC/MS (method 5): Rₜ=2.54 min., m/z=511.1 [M+H]⁺.

### Example 22

### Ethyl (1-{[8-chloro-6-(2,3-dimethoxyphenyl)-1-isopropyl-4H,6H-[1,2,4]triazolo[4,3-a][4,1]benzoxazepin-4-yl]acetyl}piperidin-4-yl)acetate

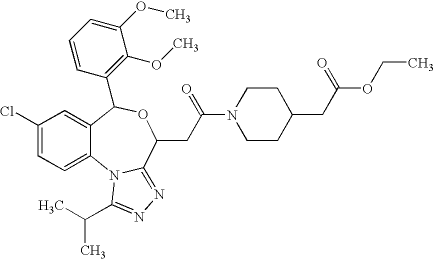

118 mg (0.23 mmol) of PyBOP and 59 mg (0.45 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 47 mg (0.23 mmol) of ethyl 4-piperidinylacetate hydrochloride are added, and the mixture is stirred overnight. It is then evaporated to dryness, and the residue is purified by preparative HPLC. 50 mg (47% of theory) of a white solid are obtained.

¹H-NMR (300 MHz, DMSO-d₆): δ=0.98 (d, 3H), 1.17 and 1.18 (2t, 3H), 1.00-1.30 (m, 2H), 1.51 (d, 3H), 1.60-1.73 (m, 2H), 1.87-1.99 (m, 1H), 2.22 (dd, 2H), 2.57 (m, partly covered by DMSO signal), 2.98-3.14 (m, 2H), 3.49 (m, 1H), 3.80 (s, 3H), 3.69-4.09 (m, 3H), 4.29 (m, 1H), 4.78 (dd, 1H), 5.30 and 5.31 (2s, 1H), 6.62 (d, 1H), 7.12-7.26 (m, 3H), 7.72 (dd, 1H), 7.97 (d, 1H).

HPLC (method 2): Rₜ=4.84 min.

MS (ESI): m/z=611.4 and 613.4 [M+H]⁺.

### Example 23

### (1-{[8-Chloro-6-(2,3-dimethoxyphenyl)-1-isopropyl-4H,6H-[1,2,4]triazolo[4,3-a][4,1]-benzoxazepin-4-yl]acetyl}piperidin-4-yl)acetic acid

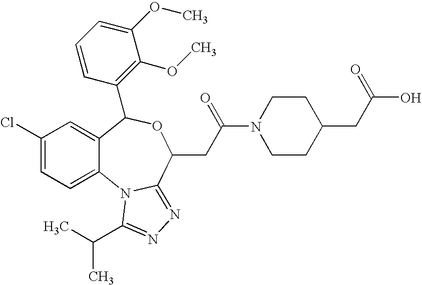

A few drops of concentrated hydrochloric acid are added to a solution of 44 mg (0.07 mmol) of the compound from Example 22 in 1.5 ml of dioxane, and the mixture is stirred at 60° C. overnight. It is then evaporated to dryness in a rotary evaporator, and the residue is purified by preparative HPLC. 18 mg (44% of theory) of the title compound are obtained.

¹H-NMR (300 MHz, DMSO-d₆): δ=0.98 (d, 3H), 1.51 (d, 3H), 1.61-1.99 (m, 3H), 2.04 (dd, 2H), 3.01-3.53 (m, partly covered by H₂O signal), 3.80 (s, 1H), 3.98 (m, 1H), 4.30 (m, 1H), 4.79 (dd, 1H), 5.30 (s, 1H), 6.61 (d, 1H), 7.12-7.26 (m, 3H), 7.73 (dd, 1H), 7.97 (d, 1H).

HPLC (method 1): Rₜ=4.32 min.

MS (ESI): m/z=583.4 and 585.4 [M+H]⁺.

### Example 24

### 1-{[8-Chloro-6-(2,3-dimethoxyphenyl)-1-isopropyl-4H,6H-[1,2,4]triazolo[4,3-a][4,1]benzoxazepin-4-yl]acetyl}piperidin-4-ol

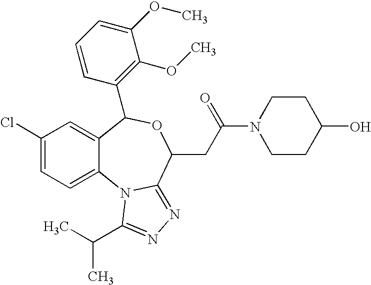

399 mg (0.77 mmol) of PyBOP and 99 mg (0.77 mmol) of N,N-diisopropylethylamine are successively added to a solution of 270 mg (0.59 mmol) of the compound from Example 3 (stereoisomer 3-3) in 15 ml of anhydrous tetrahydrofuran at room temperature. After 30 minutes, 78 mg (0.77 mmol) of 4-hydroxypiperidine are added, and the mixture is stirred at room temperature overnight. It is then evaporated to dryness and the residue is purified by preparative HPLC. 178 mg (56% of theory) of a white solid are obtained.

¹H-NMR (300 MHz, DMSO-d₆): δ=0.98 (d, 3H), 1.51 (d, 3H), 1.62-1.81 (m, 2H), 2.97-3.09 (m, 2H), 3.28 (s, 3H), 3.50 (m, 1H), 3.66-3.72 (m, 1H), 3.76-3.88 (m, 1H), 3.80 (s, 3H), 4.78 (dd, 1H), 5.30 (s, 1H), 6.62 (d, 1H), 7.11-7.26 (m, 3H), 7.73 (dd, 1H), 7.89 (d, 1H).

HPLC (method 2): Rₜ=4.13 min.

MS (ESI): m/z=541.4 and 543.4 [M+H]⁺.

The following compound is prepared in analogy to the examples described above from the appropriate starting compounds:

### Example 25

### 8-Chloro-6-(2,3-dimethoxyphenyl)-1-isopropyl-4-[2-(4-methylpiperazin-1-yl)-2-oxoethyl]-4H,6H-[1,2,4]triazolo[4,3-a][4,1]benzoxazepine

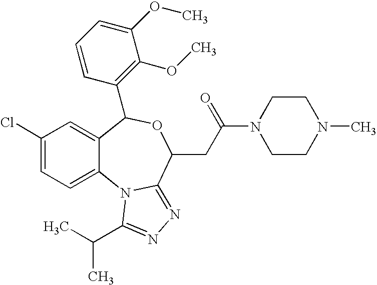

### Example 26

### 8-Chloro-6-(2,3-dimethoxyphenyl)-1-isopropyl-4-[2-(morpholin-4-yl)-2-oxoethyl]-4H,6H-[1,2,4]-triazolo[4,3-a][4,1]benzoxazepine

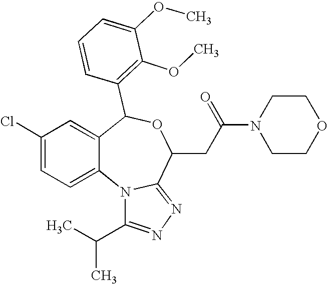

295 mg (0.57 mmol) of PyBOP and 73 mg (0.57 mmol) of N,N-diisopropylethylamine are successively added to a solution of 200 mg (0.44 mmol) of the compound from Example 3 (stereoisomer 3-3) in 10 ml of anhydrous tetrahydrofuran at room temperature. After 30 minutes, 50 mg (0.57 mmol) of morpholine are added, and the mixture is stirred overnight. It is then evaporated to dryness, and the residue is purified by preparative HPLC. 143 mg (62% of theory) of a white solid are obtained.

¹H-NMR (400 MHz, DMSO-d₆): δ=0.98 (d, 3H), 1.51 (d, 3H), 3.09 (dd, 1H), 3.37 (m, partly covered by H₂O signal), 3.42-3.62 (m, 8H), 4.80 (dd, 1H), 5.31 (s, 1H), 6.62 (d, 1H), 7.13-7.26 (m, 3H), 7.73 (dd, 1H), 7.97 (d, 1H).

HPLC (method 2): Rₜ=4.45 min.

MS (ESI): m/z=527.3 and 529.3 [M+H]⁺.

### Example 27

### 8-Chloro-6-(2,3-dimethoxyphenyl)-1-isopropyl-4-[2-(4-methylpiperidin-1-yl)-2-oxoethyl]-4H,6H-[1,2,4]triazolo[4,3-a][4,1]benzoxazepine

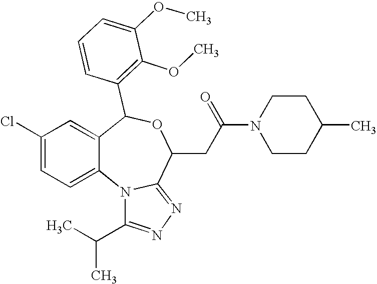

62.5 mg of PyBOP (0.120 mmol) and 21 μl of N,N-diisopropylethylamine (16 mg, 0.12 mmol) are added to 50 mg of the compound from Example 3 (0.109 mmol) in 2 ml of tetrahydrofuran and 50 μl of dimethylformamide at 0° C. The mixture is stirred at RT for 1 h and then 8.2 μl of 4-methylpiperidine (12 mg, 0.12 mmol) are added. The mixture is stirred at RT for 1 h and then the solvent is removed under reduced pressure. The residue is purified by preparative HPLC (eluent: acetonitrile/water with 0.1% formic acid, gradient 10:90→95:5). 20 mg (28% of theory) of racemic diastereomer 27-1 and 28 mg (48% of theory) of racemic diastereomer 27-2 are obtained.

### Diastereomer 27-1, Racemic:

HPLC (method 2): Rₜ=5.03 min.

MS (ESI): m/z=539.5 [M+H]⁺.

### Diastereomer 27-2, Racemic:

HPLC (method 1): Rₜ=4.80 min.

MS (ESI): m/z=539.5 [M+H]⁺.

### Example 28

### [8-Chloro-6-(2,3-dimethoxyphenyl)-1-ethyl-4H,6H-[1,2,4]triazolo[4,3-a][4,1]benzoxazepin-4-yl]-acetic acid

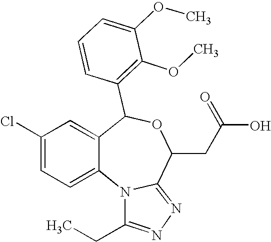

200 μl of concentrated hydrochloric acid are added to 25 mg of the compound from Example 5 (0.053 mmol) in 3 ml of dioxane, and the mixture is stirred at 80° C. overnight. The solvent is removed under reduced pressure, and the residue is purified by preparative HPLC (eluent: acetonitrile/water with 0.1% formic acid, gradient 10:90→95:5). 9 mg (34% of theory) of the title compound are obtained.

¹H-NMR (400 MHz, DMSO-d₆): δ=1.18 (t, 3H), 2.93-3.06 (m, 2H), 3.07-3.28 (m, 3H), 3.34 (s, 3H, underneath H₂O signal), 3.82 (s, 3H), 4.75 (dd, 1H), 5.38 (s, 1H), 6.64 (d, 1H), 7.13-7.18 (m, 2H), 7.19-7.26 (m, 1H), 7.74 (dd, 1H), 7.92 (d, 1H).

LC/MS (method 5): Rₜ=2.19 min., m/z=444.1 [M+H]⁺.

The following compounds are prepared in analogy to the examples described above from the appropriate starting compounds:

### Example 29

### [8-Chloro-1-isopropyl-6-(2-methoxyphenyl)-4H,6H-[1,2,4]triazolo[4,3-a][4,1]benzoxazepin-4-yl]-acetic acid

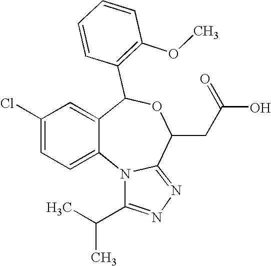

### Example 30

### {6-[2,3-bis(Dimethylamino)phenyl]-8-chloro-1-isopropyl-4H,6H-[1,2,4]triazolo[4,3-a][4,1]-benzoxazepin-4-yl}acetic acid

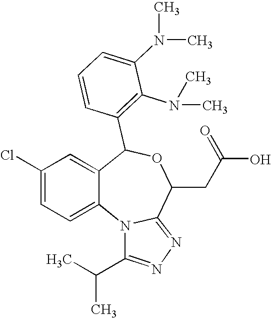

### Example 31

### 1-{[8-Chloro-6-(2,3-dimethoxyphenyl)-1-isopropyl-4H,6H-[1,2,4]triazolo[4,3-a][4,1]benzoxazepin-4-yl]acetyl}-D-proline tert-butyl ester

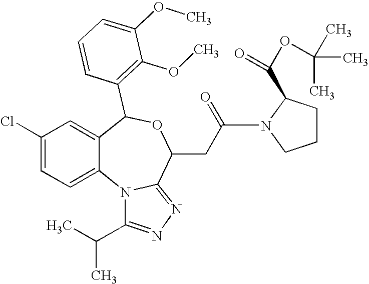

103 mg (0.2 mmol) of PyBOP and 26 mg (0.2 mmol) of N,N-diisopropylethylamine are added successively to a solution of 70 mg (0.15 mmol) of the compound from Example 3 (stereoisomer 3-3) in 5 ml of anhydrous tetrahydrofuran at room temperature. After 30 minutes, 34 mg (0.2 mmol) of D-proline tert-butyl ester are added and the mixture is stirred overnight. It is then evaporated to dryness and the residue is purified by preparative HPLC. 63 mg (68% of theory) of a solid are obtained.

¹H-NMR (400 MHz, DMSO-d₆): δ=0.99 (d, 3H), 1.33 (s, 6H), 1.39 (s, 3H), 1.74-1.92 (m, 3H), 2.04-2.28 (m, 2H), 3.12 (dd, 1H), 3.22 (dd, 1H), 3.34 (s, 3H, partly covered by H₂O signal), 3.50 (pseudo-quint, 1H), 3.58-3.69 (m, 1H), 3.81 (s, 3H), 4.18 (dd, 1H), 4.78 (dd, 1H), 5.32 (s, 1H), 6.62 (d, 1H), 7.13 (d, 2H), 7.17-7.26 (m, 1H), 7.73 (dd, 1H), 7.92 (d, 1H).

HPLC (method 2): Rₜ=4.98 min.

MS (ESI): m/z=611.4 and 613.4 [M+H]⁺.

### Example 32

### 1-{[8-Chloro-6-(2,3-dimethoxyphenyl)-1-isopropyl-4H,6H-[1,2,4]triazolo[4,3-a][4,1]benzoxazepin-4-yl]acetyl}-D-proline

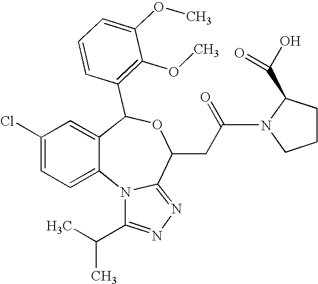

0.5 ml of trifluoroacetic acid is added to a solution of 59 mg (0.1 mmol) of the compound from Example 31 in 1 ml of dichloromethane, and the mixture is stirred at room temperature for 2 hours. The mixture is then evaporated to dryness and extracted with ethyl acetate in the presence of water. The crude product obtained in this way is purified by preparative HPLC. 33 mg (62% of theory) of the title compound are obtained.

¹H-NMR (300 MHz, DMSO-d₆): δ=0.98 (d, 3H), 1.52 (d, 3H), 1.77-2.32 (m, 4H), 3.13 (dd, 1H), 3.24 (dd, 1H), 3.43-3.67 (m, 3H), 4.23 (dd, 1H), 4.79 (m, 1H), 5.32 (s, 1H), 6.62 (dd, 1H), 7.13-7.27 (m, 3H), 7.73 (dd, 1H), 7.93 (d, 1H), 12.78 (1H).

HPLC (method 1): Rₜ=4.34 min.

MS (ESI): m/z=555.4 and 557.4 [M+H]⁺.

### Example 33

### Ethyl 1-{[8-chloro-6-(2,3-dimethoxyphenyl)-1-isopropyl-4H,6H-[1,2,4]triazolo[4,3-a][4,1]benzoxazepin-4-yl]acetyl}piperidine-3-carboxylate

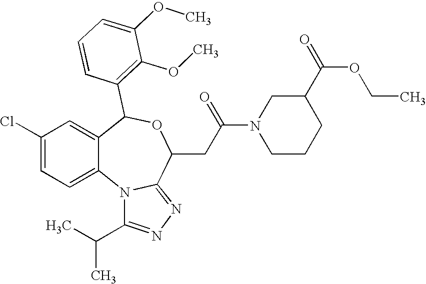

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 36 mg (0.23 mmol) of ethyl piperidine-3-carboxylate (as racemate) are added, and the mixture is stirred overnight. It is then evaporated to dryness, and the residue is purified by preparative HPLC. 80 mg (77% of theory) of a white solid are obtained.

¹H-NMR (400 MHz, DMSO-d₆): δ=0.98 (d, 3H), 1.17 and 1.18 (2t, 3H), 1.31-1.42 (m, 1H), 1.51 (d, 3H), 1.53-1.78 (m, 2H), 1.85-1.99 (m, 1H), 2.27-2.37 and 2.53-2.59 (2m, 1H), 2.72 and 2.83 (2dd, 1H), 3.01-3.22 (m, 2H), 3.32 (s, 3H), 3.80 (s, 3H), 3.79-3.91 (m, 1H), 4.07 (q, 2H), 4.27 and 4.37 (2m, 1H), 4.79 (m, 1H), 5.31 (s, 1H), 6.62 (s, 1H), 7.12-7.24 (m, 3H), 7.73 (d, 1H), 7.97 (d, 1H).

HPLC (method 2): Rₜ=4.81 min.

MS (ESI): m/z=597.4 and 599.4 [M+H]⁺.

### Example 34

### 1-{[8-Chloro-6-(2,3-dimethoxyphenyl)-1-isopropyl-4H,6H-[1,2,4]triazolo[4,3-a][4,1]benzoxazepin-4-yl]acetyl}piperidine-3-carboxylic acid

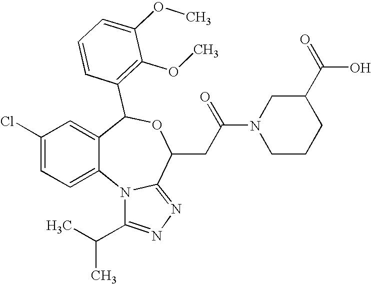

A solution of 62 mg (0.1 mmol) of the compound from Example 33 in 2.5 ml of dioxane is mixed with 1 ml of concentrated hydrochloric acid and heated at 60° C. for 4 hours. The reaction mixture is then evaporated to dryness, and the residue is purified by preparative HPLC. 44 mg (74% of theory) of a white solid are obtained.

¹H-NMR (300 MHz, DMSO-d₆): δ=0.98 (d, 3H), 1.14-1.38 (m, 1H), 1.51 (d, 3H), 1.53-1.72 (m, 2H), 1.87-2.00 (m, 1H), 2.18-2.30 and 2.59-2.77 (2m, 1H), 3.00-3.13 (m, 1H), 3.38-3.54 (m, 4H), 3.81 (s, 3H), 3.75-3.97 (m, 1H), 4.33 and 4.41 (2m, 1H), 4.78 (m, 1H), 5.31 (s, 1H), 6.62 (d, 1H), 7.11-7.27 (m, 3H), 7.73 (dd, 1H), 7.97 (d, 1H).

HPLC (method 2): Rₜ=4.40 min.

MS (ESI): m/z=569.3 and 571.3 [M+H]⁺.

### Example 35

### Ethyl 1-{[8-chloro-6-(2,3-dimethoxyphenyl)-1-isopropyl-4H,6H-[1,2,4]triazolo[4,3-a][4,1]benzoxazepin-4-yl]acetyl}piperidine-2-carboxylate

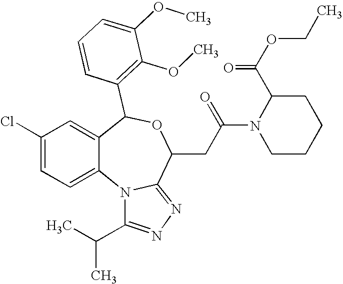

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 36 mg (0.23 mmol) of ethyl piperidine-2-carboxylate (as racemate) are added, and the mixture is stirred overnight. It is then evaporated to dryness, and the residue is purified by preparative HPLC. 83 mg (80% of theory) of a white solid are obtained.

HPLC (method 2): Rₜ=4.96 min.

MS (ESI): m/z=597.4 and 599.4 [M+H]⁺.

### Example 36

### 1-{[8-Chloro-6-(2,3-dimethoxyphenyl)-1-isopropyl-4H,6H-[1,2,4]triazolo[4,3-a][4,1]benzoxazepin-4-yl]acetyl}piperidine-2-carboxylic acid

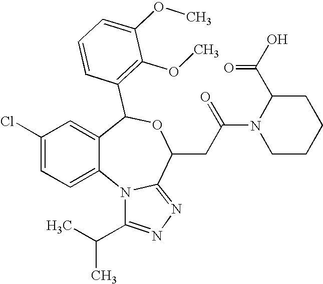

A solution of 62 mg (0.1 mmol) of the compound from Example 35 in 2.5 ml of dioxane is mixed with 1 ml of concentrated hydrochloric acid and heated at 60° C. for 10 hours. The reaction mixture is then evaporated to dryness, and the residue is purified by preparative HPLC. In this case, the two diastereomeric products are obtained separately from one another in each case as white solids.

### Diastereomer 36-1:

11 mg (19% of theory)

¹H-NMR (300 MHz, DMSO-d₆): δ=0.98 (d, 2H), 1.22-1.32 (m, 2H), 1.52 (d, 3H), 1.47-1.58 (m, 1H), 1.62-1.71 (m, 2H), 2.10 (m, 2H), 2.98-3.20 (m, 2H), 3.47-3.55 (m, 2H), 3.81 (s, 3H), 3.97 (m, 1H), 4.75-4.87 (m, 1H), 4.98 (m, 1H), 5.29 (s, 1H), 6.61 (s, 1H), 7.11-7.29 (m, 3H), 7.73 (dd, 1H), 7.97 (d, 1H), 12.70 (br. s, 1H).

HPLC (method 2): Rₜ=4.48 min.

MS (ESI): m/z=569.3 and 571.3 [M+H]⁺.

### Diastereomer 36-2:

11 mg (19% of theory)

¹H-NMR (300 MHz, DMSO-d₆): δ=0.98 (d, 2H), 1.20-1.38 (m, 2H), 1.51 (d, 3H), 1.55-1.70 (m, 3H), 2.07-2.28 (m, 2H), 3.80 (s, 3H), 4.08 (m, 1H), 4.78 (m, 1H), 5.01 (m, 1H), 5.31 (s, 1H), 6.62 (d, 1H), 7.12-7.23 (m, 3H), 7.73 (dd, 1H), 7.94 (d, 1H).

HPLC (method 2): Rₜ=4.61 min.

MS (ESI): m/z=569.3 and 571.3 [M+H]⁺.

### Example 37

### 1-{[8-Chloro-6-(2,3-dimethoxyphenyl)-1-isopropyl-4H,6H-[1,2,4]triazolo[4,3-a][4,1]benzoxazepin-4-yl]acetyl}pyrrolidin-2-one

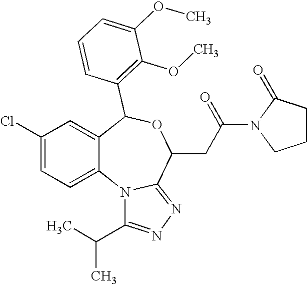

80 μl of thionyl chloride (1.09 mmol) and 2 drops of N,N-dimethylformamide are successively added to a solution of 100 mg (0.22 mmol) of the compound from Example 3 (stereoisomer 3-3) in 2 ml of 1,2-dichloroethane. The mixture is stirred at room temperature for 5 hours. The mixture is then evaporated to dryness, and the residue is taken up in a little toluene and again concentrated. The residue is dissolved in 2 ml of anhydrous tetrahydrofuran and, after addition of 76111 (0.44 mmol) of N,N-diisopropylethylamine and 18 μl of 2-pyrrolidone, stirred at room temperature overnight. After this time, the mixture is evaporated to dryness, and the residue is purified by preparative HPLC. 35 mg (30% of theory) of a white solid are obtained.

¹H-NMR (300 MHz, DMSO-d₆): δ=0.98 (d, 3H), 1.52 (d, 3H), 1.95 (quint, 2H), 2.58 (dt, 2H), 3.46-3.56 (m, 2H), 3.60-3.67 (m, 2H), 3.82 (s, 3H), 3.87 (dd, 1H), 4.86 (dd, 1H), 5.32 (s, 1H), 6.63 (d, 1H), 7.12-2.72 (m, 3H), 7.74 (dd, 1H), 7.98 (d, 1H).

HPLC (method 2): Rₜ=4.63 min.

MS (ESI): m/z=525.3 and 527.3 [M+H]⁺.

### Example 38

### Methyl 1-{[8-chloro-6-(2,3-dimethoxyphenyl)-1-isopropyl-4H,6H-[1,2,4]triazolo[4,3-a][4,1]benzoxazepin-4-yl]acetyl}piperidine-4-carboxylate

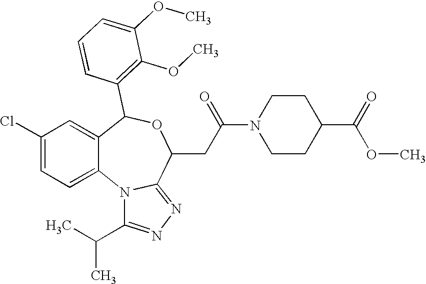

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 33 mg (0.23 mmol) of methyl piperidine-4-carboxylate are added, and the mixture is stirred overnight. It is then evaporated to dryness, and the residue is purified by preparative HPLC. 64 mg (63% of theory) of a white solid are obtained.

¹H-NMR (300 MHz, DMSO-d₆): δ=0.98 (d, 3H), 1.30-1.43 (m, 1H), 1.51 (d, 3H), 1.48-1.67 (m, 1H), 1.77-1.91 (m, 2H), 2.57-2.80 (m, 2H), 3.01-3.22 (m, 2H), 3.32-3.53 (m, 2H), 3.61 and 3.63 (2s, 3H), 3.81 (s, 3H), 3.97 (m, 1H), 4.18 (m, 1H), 4.79 (t, 1H), 5.30 (s, 1H), 6.62 (d, 1H), 7.12-7.26 (m, 3H), 7.72 (dd, 1H), 7.97 (d, 1H).

HPLC (method 2): Rₜ=4.60 min.

MS (ESI): m/z=583.3 and 585.3 [M+H]⁺.

### Example 39

### 1-{[8-Chloro-6-(2,3-dimethoxyphenyl)-1-isopropyl-4H,6H-[1,2,4]triazolo[4,3-a][4,1]benzoxazepin-4-yl]acetyl}piperidine-4-carboxylic acid

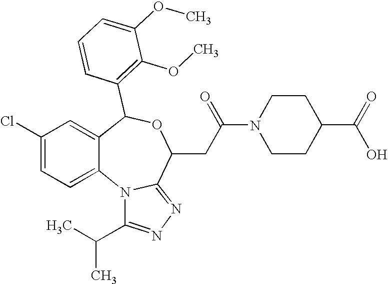

A solution of 56 mg (0.1 mmol) of the compound from Example 38 in 1.5 ml of dioxane is mixed with 0.5 ml of concentrated hydrochloric acid and heated at 60° C. overnight. The reaction mixture is then evaporated to dryness, and the residue is purified by preparative HPLC. 20 mg (36% of theory) of a white solid are obtained.

¹H-NMR (300 MHz, DMSO-d₆): δ=0.98 (d, 3H), 1.29-1.39 (m, 1H), 1.51 (d, 3H), 1.48-1.62 (m, 1H), 1.76-1.89 (m, 2H), 2.66-2.80 (m, 2H), 2.97-3.37 (m, 3H), 3.38-3.54 (m, 2H), 3.81 (s, 3H), 3.94 (m, 1H), 4.17 (m, 1H), 4.71 and 4.79 (2t, 1H), 5.30 and 5.33 (2s, 1H), 6.62 (d, 1H), 7.12-7.27 (m, 3H), 7.73 (dd, 1H), 7.98 (d, 1H), 12.32 (br. s, 1H).

HPLC (method 2): Rₜ=4.31 min.

MS (ESI): m/z=569.3 and 571.3 [M+H]⁺.

### Example 40

### 1-{[8-Chloro-6-(2,3-dimethoxyphenyl)-1-isopropyl-4H,6H-[1,2,4]triazolo[4,3-a][4,1]benzoxazepin-4-yl]acetyl}-L-proline

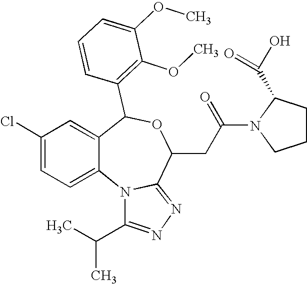

The title compound is obtained in analogy to the way described in Examples 31 and 32 starting from the compound from Example 3 (stereoisomer 3-3) and L-proline tert-butyl ester.

¹H-NMR (300 MHz, DMSO-d₆): δ=0.98 (d, 3H), 1.51 (d, 3H), 1.83-2.00 (m, 3H), 2.09-2.19 (m, 1H), 3.04 (dd, 1H), 3.48-3.53 (m, 3H), 3.63-3.71 (m, 2H), 3.80 (s, 3H), 4.18 (dd, 1H), 4.76 (t, 1H), 5.31 (s, 1H), 6.61 (dd, 1H), 7.12 (dd, 1H), 7.21 (dd, 1H), 7.31 (dd, 1H), 7.72 (dd, 1H), 7.96 (d, 1H), 12.48 (br. s, 1H).

HPLC (method 1): Rₜ=4.19 min.

MS (ESI): m/z=555.4 and 557.4 [M+H]⁺.

### Example 41

### 8-Chloro-6-(2,3-dimethoxyphenyl)-1-isopropyl-4-[2-oxo-2-(1,3-thiazolidin-3-yl)ethyl]-4H,6H-[1,2,4]triazolo[4,3-a][4,1]benzoxazepine

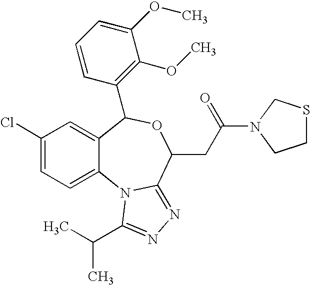

222 mg (0.43 mmol) of PyBOP and 74 μl (0.43 mmol) of N,N-diisopropylethylamine are successively added to a solution of 150 mg (0.33 mmol) of the compound from Example 3 (stereoisomer 3-3) in 5 ml of anhydrous tetrahydrofuran at room temperature. After 30 minutes, 34 μl (0.43 mmol) of thiazolidine are added, and the mixture is stirred overnight. It is then evaporated to dryness, and the residue is purified by preparative HPLC. 134 mg (77% of theory) of a white solid are obtained.

¹H-NMR (300 MHz, DMSO-d₆): δ=0.98 (d, 3H), 1.51 (d, 3H), 2.99 (t, 1H), 3.07-3.17 (m, 2H), 3.31 (s, 3H), 3.64-3.79 (m, 3H), 3.81 (s, 3H), 3.82-3.91 (m, 1H), 4.42 (m, 1H), 4.71 (dd, 1H), 4.79 (t, 1H), 5.32 (s, 1H), 6.62 (d, 1H), 7.12-2.27 (m, 3H), 7.73 (dd, 1H), 7.97 (d, 1H).

HPLC (method 2): Rₜ=4.57 min.

MS (ESI): m/z=529.4 and 531.4 [M+H]⁺.

### Example 42

### ((2R)-1-{[8-Chloro-6-(2,3-dimethoxyphenyl)-1-isopropyl-4H,6H-[1,2,4]triazolo[4,3-a][4,1]benzoxazepin-4-yl]acetyl}pyrrolidin-2-yl)methanol

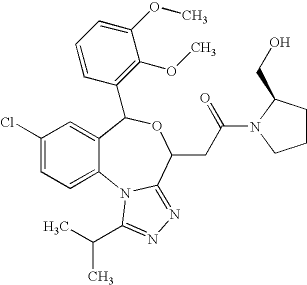

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 23 mg (0.23 mmol) of (R)-2-(hydroxymethyl)pyrrolidine are added, and the mixture is stirred overnight. It is then evaporated to dryness, and the residue is purified by preparative HPLC. 74 mg (76% of theory) of a white solid are obtained.

¹H-NMR (300 MHz, DMSO-d₆): δ=0.98 (d, 3H), 1.52 (d, 3H), 1.73-1.94 (m, 4H), 2.99-3.12 (m, 1H), 3.18-3.37 (m, 2H), 3.41-3.60 (m, 4H), 3.81 (s, 3H), 3.92 and 4.22 (2m, 1H), 4.69, 4.79 and 4.92 (3m, 2H), 5.31 and 5.32 (2s, 1H), 6.62 (dd, 1H), 7.12-7.27 (m, 3H), 7.73 (dd, 1H), 7.98 (d, 1H).

HPLC (method 2): Rₜ=4.35 min.

MS (ESI): m/z=541.4 and 543.3 [M+H]⁺.

### Example 43

### tert-Butyl 1-{[8-chloro-6-(2,3-dimethoxyphenyl)-1-isopropyl-4H,6H-[1,2,4]triazolo[4,3-a][4,1]benzoxazepin-4-yl]acetyl}pyrrolidine-3-carboxylate

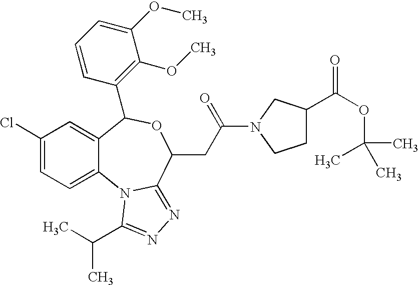

148 mg (0.28 mmol) of PyBOP and 37 mg (0.28 mmol) of N,N-diisopropylethylamine are successively added to a solution of 100 mg (0.22 mmol) of the compound from Example 3 (stereoisomer 3-3) in 5 ml of anhydrous tetrahydrofuran at room temperature. After 30 minutes, 97 mg (0.28 mmol) of tert-butyl 3-pyrrolidinecarboxylate (as racemate) are added, and the mixture is stirred overnight. It is then evaporated to dryness, and the residue is purified by preparative HPLC. 91 mg (68% of theory) of a white solid are obtained.

¹H-NMR (300 MHz, DMSO-d₆): δ=0.98 (d, 3H), 1.41 (s, 9H), 1.52 (d, 3H), 1.39-2.19 (m, 2H), 2.97-3.19 (m, 3H), 3.48-3.71 (m, 3H), 3.82 (s, 3H), 4.78 (t, 1H), 5.31 (s, 1H), 6.62 (dd, 1H), 7.12-7.27 (m, 3H), 7.74 (dd, 1H), 7.97 (d, 1H).

HPLC (method 2): Rₜ=4.95 min.

MS (ESI): m/z=611.5 and 613.5 [M+H]⁺.

### Example 44

### 1-{[8-Chloro-6-(2,3-dimethoxyphenyl)-1-isopropyl-4H,6H-[1,2,4]triazolo[4,3-a][4,1]benzoxazepin-4-yl]acetyl}pyrrolidine-3-carboxylic acid

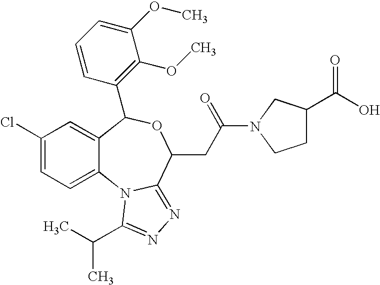

A solution of 69 mg (0.11 mmol) of the compound from Example 43 in 1.5 ml of dichloromethane is mixed with 0.7 ml of trifluoroacetic acid and stirred at room temperature for one hour. It is then evaporated to dryness, and the resulting residue is purified by preparative HPLC. 42 mg (67% of theory) of a white solid are obtained.

¹H-NMR (400 MHz, DMSO-d₆): δ=0.98 (d, 3H), 1.51 (s, 9H), 1.52 (d, 3H), 1.92-2.21 (m, 2H), 2.97-3.20 (m, 3H), 3.42-3.71 (m, 4H), 3.81 (s, 3H), 4.77 (m, 1H), 5.31 (s, 1H), 6.62 (dd, 1H), 7.12-7.26 (m, 3H), 7.73 (dd, 1H), 7.95 (d, 1H), 12.52 (br. s, 1H).

HPLC (method 2): Rₜ=4.21 min.

MS (ESI): m/z=555 and 557 [M+H]⁺.

### Example 45

### 1-{[8-Chloro-6-(2,3-dimethoxyphenyl)-1-isopropyl-4H,6H-[1,2,4]triazolo[4,3-a][4,1]benzoxazepin-4-yl]acetyl}pyrrolidin-3-ol

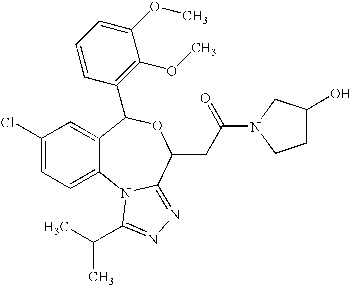

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 20 mg (0.23 mmol) of 3-hydroxypyrrolidine (as racemate) are added, and the mixture is stirred overnight. It is then evaporated to dryness, and the residue is purified by preparative HPLC. 76 mg (82% of theory) of a white solid are obtained.

¹H-NMR (300 MHz, DMSO-d₆): δ=0.98 (d, 3H), 1.51 (d, 3H), 1.70-2.01 (m, 2H), 2.90-3.08 (m, 1H), 3.42-3.74 (m, 4H), 3.81 (s, 3H), 4.24 and 4.32 (2m, 1H), 4.78 (t, 1H), 5.31 (s, 1H), 6.62 (d, 1H), 7.12-7.27 (m, 3H), 7.73 (dd, 1H), 7.97 (d, 1H).

HPLC (method 2): Rₜ=4.15 min.

MS (ESI): m/z=527.3 and 529.3 [M+H]⁺.

### Example 46

### 8-Chloro-6-(2,3-dimethoxyphenyl)-4-[2-(1,1-dioxo-1,3-thiazolidin-3-yl)-2-oxoethyl]-1-isopropyl-4H,6H-[1,2,4]triazolo[4,3-a][4,1]benzoxazepine

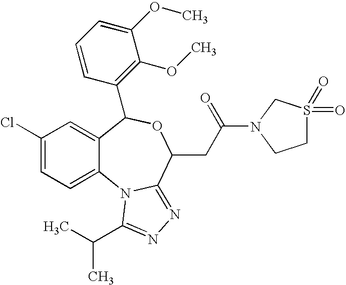

A solution of 36 mg (0.23 mmol) of potassium permanganate in a little water is added to a solution of 80 mg (0.15 mmol) of the compound from Example 41 in 3 ml of a mixture of glacial acetic acid and water (5:1) at room temperature. After stirring for one hour, 30 ml of water are added, and the mixture is extracted twice with 50 ml of ethyl acetate each time. The combined organic extracts are washed successively with sodium bisulphite solution, water and saturated brine. Drying over anhydrous sodium sulphate is followed by filtration, and the solvent is stripped off in vacuo and the residue is purified by preparative HPLC. 77 mg (91% of theory) of a white solid are obtained.

¹H-NMR (400 MHz, DMSO-d₆): δ=0.99 (d, 3H), 1.51 (d, 3H), 3.39-3.57 (m, 4H), 3.72-3.86 (m, 1H), 3.81 (s, 3H), 4.18 (m, 1H), 4.48 (dd, 1H), 4.80 (m, 1H), 4.87 (dd, 1H), 5.33 (d, 1H), 6.63 (d, 1H), 7.12-7.25 (m, 3H), 7.73 (dd, 1H), 7.97 (d, 1H).

HPLC (method 2): Rₜ=4.43 min.

MS (ESI): m/z=561.4 and 563.4 [M+H]⁺.

### Example 47

### Ethyl [8-chloro-6-(2,3-dimethoxyphenyl)-1-(1-methoxy-1-methylethyl)-4H,6H-[1,2,4]triazolo[4,3-a][4,1]benzoxazepin-4-yl]acetate

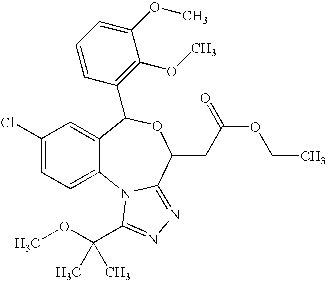

A solution of 5.6 g (12.85 mmol) of the compound from Example 8A and 2.55 g (19.27 mmol) of 2-methoxy-2-methylpropanoyl hydrazide [CAS No. 54871-29-3] in 60 ml of dioxane is heated to reflux. After 15 hours, a further 1.95 g of the hydrazide are added, and the heating under reflux is continued for a further day. The solvent is then removed in a rotary evaporator, and the residue is purified by flash chromatography on silica gel (mobile phase gradient cyclohexane/ethyl acetate 10:1→2:1). 4.38 g (66% of theory) of the title compound are obtained. In addition, 0.9 g (16% of theory) of the thioamide employed is recovered.

### Diastereomer Mixture 47-1:

HPLC (method 2): Rₜ=4.88 min. (56%) and 5.05 min. (44%)

MS (ESI): m/z=516.5 and 518.5 [M+H]⁺.

The diastereomers are separated by chromatography (Kromasil 100 C18, 5 μm, 250 mm×20 mm; eluent: 0.2% trifluoroacetic acid in water/acetonitrile (35:65); flow rate: 25 ml/min.; oven: 40° C.; UV detection: 210 nm). 2.22 g of diastereomer 47-2 and 1.69 g of diastereomer 47-3 are obtained.

### Diastereomer 47-2, Racemic:

¹H-NMR (300 MHz, DMSO-d₆): δ=0.38 (s, 3H), 1.17 (t, 3H), 1.43 (s, 3H), 3.00 (s, 3H), 3.27 (s, 3H), 3.18-3.42 (m, 2H, partly covered by H₂O signal), 3.64 (s, 3H), 4.09 (q, 2H), 4.77 (dd, 1H), 6.13 (d, 1H), 6.25 (s, 1H), 6.72 (dd, 1H), 6.84 (d, 1H), 7.62-7.72 (m, 2H), 8.02 (d, 1H).

HPLC (method 2): Rₜ=4.88 min.

MS (ESI): m/z=516 and 518 [M+H]⁺.

### Diastereomer 47-3, Racemic:

¹H-NMR (300 MHz, DMSO-d₆): δ=1.18 (t, 3H), 1.33 (s, 3H), 1.78 (s, 3H), 3.12 (dd, 1H), 3.15 (s, 3H), 3.28 (dd, 1H), 3.30 (s, 3H), 3.81 (s, 3H), 4.10 (2q, 2H), 4.69 (dd, 1H), 5.37 (s, 1H), 6.58 (d, 1H), 7.09-7.26 (m, 3H), 7.73 (dd, 1H), 7.88 (d, 1H).

HPLC (method 2): Rₜ=5.05 min.

MS (ESI): m/z=516 and 518 [M+H]⁺.

### Example 48

### [8-Chloro-6-(2,3-dimethoxyphenyl)-1-(1-methoxy-1-methylethyl)-4H,6H-[1,2,4]triazolo[4,3-a][4,1]benzoxazepin-4-yl]acetic acid

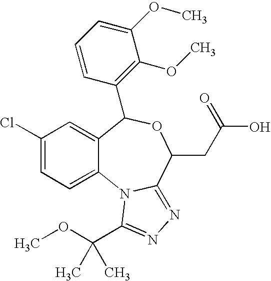

1.6 g (3.10 mmol) of the compound from Example 47-3 are dissolved in 50 ml of dioxane, mixed with 11 ml of concentrated hydrochloric acid and stirred at 60° C. overnight. The mixture is then evaporated to dryness and dried under high vacuum. 1.4 g (93% of theory) of a white solid are obtained.

¹H-NMR (400 MHz, DMSO-d₆): δ=1.34 (s, 3H), 1.78 (s, 3H), 3.03 (dd, 1H), 3.16 (s, 3H), 3.22 (dd, 1H), 3.33 (s, 3H), 3.81 (s, 3H), 4.65 (dd, 1H), 5.37 (s, 1H), 6.59 (d, 1H), 7.13 (d, 2H), 7.22 (dd, 1H), 7.74 (dd, 1H), 7.88 (d, 1H).

HPLC (method 1): Rₜ=4.43 min.

MS (ESI): m/z=488.1 and 490.1 [M+H]⁺.

### Example 49

### 8-Chloro-6-(2,3-dimethoxyphenyl)-4-[2-(1,1-dioxothiomorpholin-4-yl)-2-oxoethyl]-1-isopropyl-4H,6H-[1,2,4]triazolo[4,3-a][4,1]benzoxazepine

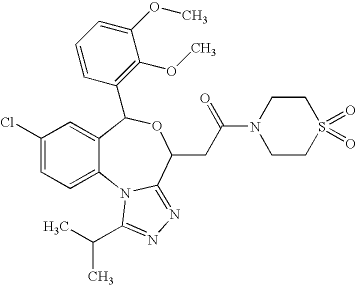

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 31 mg (0.23 mmol) of thiomorpholine S,S-dioxide are added, and the mixture is stirred overnight. It is then evaporated to dryness, and the residue is purified by preparative HPLC. 100 mg (99% of theory) of a white solid are obtained.

¹H-NMR (300 MHz, DMSO-d₆): δ=0.98 (d, 3H), 1.52 (d, 3H), 2.97-3.11 (m, 2H), 3.19 (dd, 2H), 3.46-3.57 (m, 2H), 3.80 (s, 3H), 3.78-3.89 (m, 2H), 3.99 (m, 2H), 4.79 (dd, 1H), 5.32 (s, 1H), 6.63 (d, 1H), 7.12-7.27 (m, 3H), 7.74 (dd, 1H), 7.99 (d, 1H).

HPLC (method 2): Rₜ=4.41 min.

MS (ESI): m/z=575.3 and 577.3 [M+H]⁺.

### Example 50

### 4-[2-(4-Acetylpiperazin-1-yl)-2-oxoethyl]-8-chloro-6-(2,3-dimethoxyphenyl)-1-isopropyl-4H,6H-[1,2,4]triazolo[4,3-a][4,1]benzoxazepine

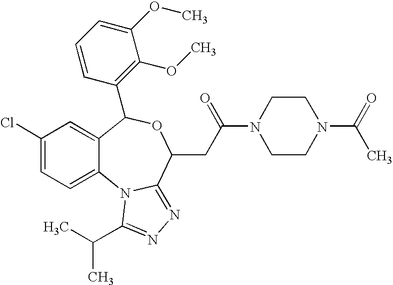

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 29 mg (0.23 mmol) of N-acetylpiperazine are added, and the mixture is stirred overnight. It is then evaporated to dryness, and the residue is purified by preparative HPLC. 63 mg (63% of theory) of a white solid are obtained.

¹H-NMR (300 MHz, DMSO-d₆): δ 0.98 (d, 3H), 1.51 (d, 3H), 2.01 (s, 3H), 3.12 (dd, 1H), 3.37-3.64 (m, 10H), 3.81 (s, 3H), 4.81 (dd, 1H), 5.31 (s, 1H), 6.62 (d, 1H), 7.12-7.27 (m, 3H), 7.74 (dd, 1H), 7.98 (d, 1H).

HPLC (method 2): Rₜ=4.19 min.

MS (ESI): m/z=568.3 and 570.3 [M+H]⁺.

### Example 51

### 1-{[8-Chloro-6-(2,3-dimethoxyphenyl)-1-isopropyl-4H,6H-[1,2,4]triazolo[4,3-a][4,1]benzoxazepin-4-yl]acetyl}-L-prolinamide

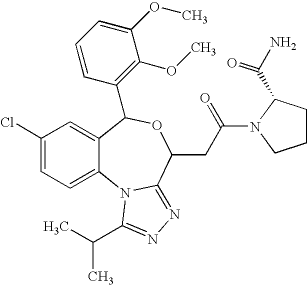

118 mg (0.23 mmol) of PyBOP and 29 mg (0.23 mmol) of N,N-diisopropylethylamine are successively added to a solution of 80 mg (0.17 mmol) of the compound from Example 3 (stereoisomer 3-3) in 5 ml of anhydrous tetrahydrofuran at room temperature. After 30 minutes, 26 mg (0.23 mmol) of L-prolinamide are added, and the mixture is stirred overnight. It is then evaporated to dryness, and the residue is purified by preparative HPLC. 82 mg (84% of theory) of a white solid are obtained.

¹H-NMR (300 MHz, DMSO-d₆): δ=0.98 (d, 3H), 1.51 (d, 3H), 1.72-2.12 (m, 4H), 3.04 (dd, 1H), 3.42-3.54 (m, 2H), 3.61-3.69 (m, 1H), 3.80 (s, 3H), 4.12 (dd, 1H), 4.72 (dd, 1H), 5.30 (s, 1H), 6.62 (d, 1H), 6.90 (br. s, 1H), 7.10-7.28 (m, 4H), 7.72 (dd, 1H), 7.95 (d, 1H).

HPLC (method 2): Rₜ=4.11 min.

MS (ESI): m/z=554.3 and 556.3 [M+H]⁺.

### Example 52

### [1-(Aminomethyl)-8-chloro-6-(2,3-dimethoxyphenyl)-4H,6H-[1,2,4]triazolo[4,3-a][4,1]benzoxazepin-4-yl]acetic acid hydrochloride

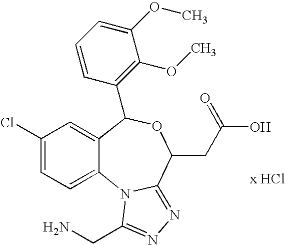

A solution of 65 mg (0.11 mmol) of the compound from Example 11A-3 in 5 ml of dioxane is mixed with 0.2 ml of concentrated hydrochloric acid and heated at 60° C. overnight. The reaction mixture is then evaporated to dryness in a rotary evaporator, and the residue is purified by preparative HPLC. 36 mg (65% of theory) of the title compound are obtained.

¹H-NMR (400 MHz, DMSO-d₆): δ=3.01 (dd, 1H), 3.23 (dd, 1H), 3.37 (s, 3H), 3.82 (s, 3H), 4.23 (m, 2H), 4.78 (t, 1H), 5.47 (s, 1H), 6.67 (d, 1H), 7.12-7.24 (m, 3H), 7.75 (dd, 1H), 8.04 (d, 1H), 8.70 (broad, 4H).

HPLC (method 2): Rₜ=3.77 min.

MS (ESI): m/z=445.2 and 447.2 [M+H]⁺.

### Example 53

### Ethyl [8-chloro-6-(2,3-dimethoxyphenyl)-1-(2-methoxyethyl)-4H,6H-[1,2,4]triazolo[4,3-a][4,1]-benzoxazepin-4-yl]acetate (racemic diastereomer)

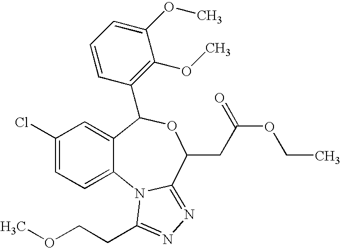

500 mg (1.15 mmol) of the compound from Example 8A and 271 mg (2.29 mmol) of 3-methoxypropanohydrazide are mixed with 5 ml of dioxane and stirred under reflux for 48 h. The residue after removal of the solvent in vacuo is purified by preparative HPLC (eluent: acetonitrile/water, gradient 20:80→80:20). 154 mg (26% of theory) of the title compound are obtained.

¹H-NMR (300 MHz, DMSO-d₆): δ=1.18 (t, 3H), 3.04-3.25 (m, 4H), 3.17 (s, 3H), 3.38 (s, 3H), 3.65 (t, 2H), 3.82 (s, 3H), 4.09 (q, 2H), 4.80 (dd, 1H), 5.42 (s, 1H), 6.64 (d, 1H), 7.06-7.25 (m, 3H), 7.74 (dd, 1H), 7.91 (d, 1H).

HPLC (method 2): Rₜ=4.71 min.

MS (ESI): m/z=502 [M+H]⁺.

### Example 54

### [8-Chloro-6-(2,3-dimethoxyphenyl)-1-(2-methoxyethyl)-4H,6H-[1,2,4]triazolo[4,3-a][4,1]benzoxazepin-4-yl]acetic acid (racemic diastereomer)

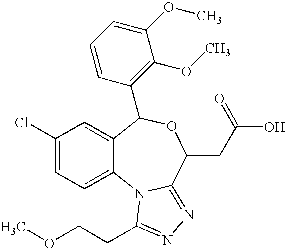

130 mg (0.26 mmol) of the compound from Example 53 are dissolved in 5.2 ml of dioxane, and five drops of concentrated hydrochloric acid are added. The mixture is stirred at room temperature for 24 hours, the solvent is removed in vacuo, and the residue is purified by preparative HPLC (eluent: acetonitrile/water, gradient 20:80→80:20). 50 mg (41% of theory) of the title compound are obtained.

¹H-NMR (400 MHz, DMSO-d₆): δ=3.00 (dd, 1H), 3.17 (s, 3H), 3.19-3.34 (m, 3H), 3.38 (s, 3H), 3.61-3.65 (m, 2H), 3.82 (s, 3H), 4.75 (dd, 1H), 5.43 (s, 1H), 6.64 (d, 1H), 7.14-7.25 (m, 3H), 7.74 (dd, 1H), 7.92 (d, 1H).

HPLC (method 2): Rₜ=4.26 min.

MS (ESI): m/z=474 [M+H]⁺.

### Example 55

### 8-Chloro-6-(2,3-dimethoxyphenyl)-1-(2-methoxyethyl)-4-(2-oxo-2-piperidin-1-ylethyl)-4H,6H-[1,2,4]triazolo[4,3-a][4,1]benzoxazepine (racemic diastereomer)

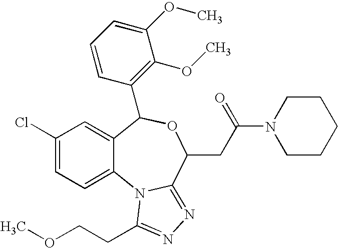

31 mg of PyBOP (0.06 mmol) and 8 mg of N,N-diisopropylethylamine (0.06 mmol) are added to

26 mg (0.06 mmol) of the compound from Example 54 in 1 ml of N,N-dimethylformamide. After stirring at room temperature for 1 h, 5 mg of piperidine (0.06 mmol) are added. After stirring at room temperature for 16 h, the solvent is removed under reduced pressure. The residue is purified by preparative HPLC (eluent: acetonitrile/water, gradient 20:80→80:20). 28 mg (88% of theory) of the title compound are obtained.

¹H-NMR (300 MHz, DMSO-d₆): δ=1.40-1.57 (m, 8H), 3.02-3.09 (m, 1H), 3.17 (s, 3H), 3.25-3.34 (m, 3H), 3.36 (s, 3H), 3.43-3.54 (m, 2H), 3.64 (t, 2H), 3.81 (s, 3H), 4.83 (dd, 1H), 5.43 (s, 1H), 6.65 (d, 1H), 7.12-7.25 (m, 3H), 7.73 (dd, 1H), 7.92 (d, 1H).

HPLC (method 2): Rₜ=4.65 min.

MS (ESI): m/z=541 [M+H]⁺.

### Example 56

### Ethyl [8-chloro-6-(2,3-dimethoxyphenyl)-1-(1-hydroxy-1-methylethyl)-4H,6H-[1,2,4]triazolo[4,3-a][4,1]benzoxazepin-4-yl]acetate

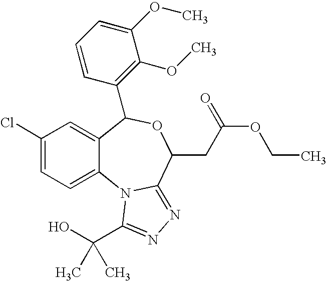

1.2 g (2.06 mmol) of the compound from Example 8A and 365 mg (3.10 mmol) of 2-hydroxy-2-methylpropanoyl hydrazide are mixed with 18 ml of dioxane and heated in an autoclave at 140° C. overnight. The residue after removal of the solvent in vacuo is purified by preparative HPLC. 587 mg of a diastereomeric mixture are obtained.

### Diastereomer Mixture 56-1:

HPLC (method 1): Rₜ=4.42 min. (55%) and 4.57 min. (35%)

MS (ESI): m/z=502.3 and 504.3 [M+H]⁺.

The diastereomers are separated by chromatography (Kromasil 100 C18, 5 μm, 250 mm×20 mm; eluent: 0.2% trifluoroacetic acid in water/acetonitrile (60:40); flow rate: 25 ml/min.; oven: 22° C.; UV detection: 210 nm). 118 mg of diastereomer 56-2 and 141 mg of diastereomer 56-3 are obtained.

### Diastereomer 56-2, Racemic:

LC/MS (method 3): 2.12 min., m/z=502.1 [M+H]⁺.

### Diastereomer 56-3, Racemic:

LC/MS (method 3): 2.24 min., m/z=502.1 [M+H]⁺.

¹H-NMR (300 MHz, DMSO-d₆): δ=1.18 (t, 3H), 1.25 (s, 3H), 1.81 (s, 3H), 3.05 (dd, 1H), 3.21-3.27 (m, 1H), 3.31 (s, 3H), 3.81 (s, 3H), 4.08 (q, 2H), 4.66 (dd, 1H), 5.35 (s, 1H), 6.58 (d, 1H), 7.12-7.26 (m, 3H), 7.75 (dd, 1H), 8.27 (d, 1H).

HPLC (method 2): Rₜ=4.68 min.

MS (ESI): m/z=502 [M+H]⁺.

### Example 57

### [8-Chloro-6-(2,3-dimethoxyphenyl)-1-(1-hydroxy-1-methylethyl)-4H,6H-[1,2,4]triazolo[4,3-a][4,1]benzoxazepin-4-yl]acetic acid

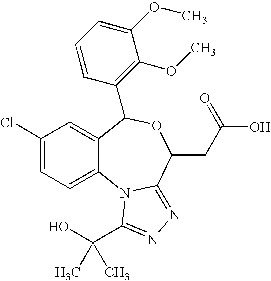

32 mg (0.06 mmol) of the compound from Example 56-3 are dissolved in 6 ml of dioxane, and four drops of concentrated hydrochloric acid are added. The mixture is stirred at room temperature for 24 hours, the solvent is removed in vacuo, and the residue is purified by preparative HPLC (eluent: acetonitrile/water, gradient 20:80→80:20). 7 mg (23% of theory) of the title compound are obtained.

### Racemate 57-1:

¹H-NMR (400 MHz, CDCl₃): δ=1.41 (s, 3H), 1.96 (s, 3H), 3.15-3.23 (m, 2H), 3.42 (s, 3H), 3.86 (s, 3H), 4.69-4.78 (m, 1H), 5.50 (s, 1H), 6.77 (d, 1H), 6.96 (d, 1H), 7.16-7.26 (m, 2H), 7.75 (dd, 1H), 8.27 (d, 1H).

HPLC (method 2): Rₜ=4.09 min.

MS (ESI): m/z=474 [M+H]⁺.

The enantiomers are separated by preparative HPLC on a chiral phase [Agilent 1100 with DAD detection; column: KBD 6175, 250 mm×20 mm, 10 μm, based on the selector poly(N-methacryloyl-L-leucine-d-menthylamide); eluent: isohexane/ethyl acetate 2:3; flow rate: 25 ml/min.; oven: 24° C.; UV detection: 254 nm]. Enantiomer 57-2 is the enantiomer with the shorter retention time.

### Enantiomer 57-2:

Starting from 312 mg (84% purity) of the racemic diastereomer 57-1 40 mg of the enantiomer 57-2 are isolated.

HPLC (method 2): Rₜ=4.09 min.

MS (ESI): m/z=474 [M+H]⁺.

¹H-NMR (300 MHz, DMSO-d₆): δ=1.27 (s, 3H), 1.81 (s, 3H), 3.03 (dd, 1H), 3.19-3.27 (m, 1H), 3.25 (s, 3H), 3.81 (s, 3H), 4.62 (dd, 1H), 5.34 (s, 1H), 6.77 (d, 1H), 7.13-7.26 (m, 3H), 7.74 (dd, 1H), 8.27 (d, 1H).

### Example 58

### Ethyl [8-chloro-6-(2,3-dimethoxyphenyl)-1-(methoxymethyl)-4H,6H-[1,2,4]triazolo[4,3-a][4,1]benzoxazepin-4-yl]acetate (racemic diastereomer)

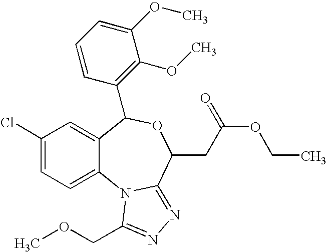

500 mg (1.15 mmol) of the compound from Example 8A and 239 mg (2.29 mmol) of 2-methoxyacetyl hydrazide are mixed with 5 ml of dioxane and stirred under reflux for 48 h. The residue after removal of the solvent in vacuo is purified by preparative HPLC (eluent: acetonitrile/water, gradient 20:80→80:20). 115 mg (19% of theory, 91% purity) of the title compound are obtained.

HPLC (method 2): Rₜ=4.77 min.

MS (ESI): m/z=488 [M+H]⁺.

### Example 59

### [8-Chloro-6-(2,3-dimethoxyphenyl)-1-(methoxymethyl)-4H,6H-[1,2,4]triazolo[4,3-a][4,1]benzoxazepin-4-yl]acetic acid (racemic diastereomer)

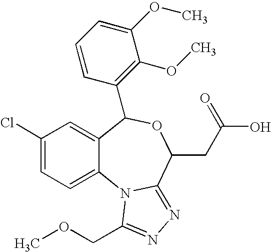

100 mg (0.20 mmol) of the compound from Example 58 are dissolved in 4 ml of dioxane and four drops of concentrated hydrochloric acid are added. The mixture is stirred at 80° C. for 3 days, the solvent is removed in vacuo, and the residue is purified by preparative HPLC (eluent: acetonitrile/water, gradient 20:80→80:20). 75 mg (79% of theory) of the title compound are obtained.

¹H-NMR (300 MHz, DMSO-d₆): δ=3.04 (dd, 1H), 3.21-3.28 (m, 1H), 3.24 (s, 3H), 3.34 (s, 3H), 3.82 (s, 3H), 4.63 (d, 1H), 4.81 (dd, 1H), 5.02 (d, 1H), 5.46 (s, 1H), 6.64 (d, 1H), 7.12-7.25 (m, 3H), 7.74 (dd, 1H), 7.94 (d, 1H).

HPLC (method 2): Rₜ=4.25 min.

MS (ESI): m/z=460 [M+H]⁺.

### Example 60

### 8-Chloro-6-(2,3-dimethoxyphenyl)-1-(methoxymethyl)-4-(2-oxo-2-piperidin-1-ylethyl)-4H,6H-[1,2,4]triazolo[4,3-a][4,1]benzoxazepine (racemic diastereomer)

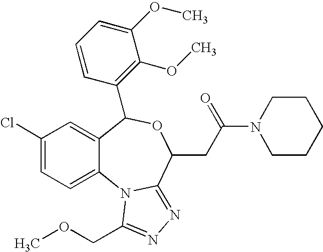

65 mg of PyBOP (0.12 mmol) and 16 mg of N,N-diisopropylethylamine (0.12 mmol) are added to 52 mg (0.11 mmol) of the compound from Example 59 in 2 ml of N,N-dimethylformamide. After stirring at room temperature for 1 h, 11 mg of piperidine (0.12 mmol) are added. After stirring at room temperature for 16 h, the solvent is removed under reduced pressure. The residue is purified by preparative HPLC (eluent: acetonitrile/water, gradient 20:80→80:20). 42 mg (70% of theory) of the title compound are obtained.

¹H-NMR (300 MHz, DMSO-d₆): δ=1.40-1.57 (m, 8H), 3.04 (dd, 1H), 3.21-3.28 (m, 1H), 3.24 (s, 3H), 3.34 (s, 3H), 3.36-3.58 (m, 2H), 3.82 (s, 3H), 4.63 (d, 1H), 4.81 (dd, 1H), 5.02 (d, 1H), 5.46 (s, 1H), 6.64 (d, 1H), 7.12-7.25 (m, 3H), 7.74 (dd, 1H), 7.94 (d, 1H).

HPLC (method 2): Rₜ=4.66 min.

MS (ESI): m/z=527 [M+H]⁺.

### Example 61

### Ethyl [8-chloro-6-(2,3-dimethoxyphenyl)-1-(2-hydroxy-1,1-dimethylethyl)-4H,6H-[1,2,4]triazolo[4,3-a][4,1]benzoxazepin-4-yl]acetate (racemic diastereomer)

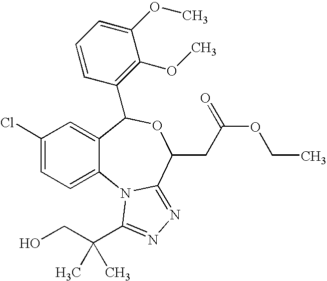

500 mg (1.15 mmol) of the compound from Example 8A and 303 mg (2.29 mmol) of 3-hydroxy-2,2-dimethylpropanoyl hydrazide are mixed with 8 ml of dioxane and stirred under reflux for 3 days. The residue after removal of the solvent in vacuo is purified by preparative HPLC (eluent: acetonitrile/water, gradient 20:80→80:20). 55 mg (9% of theory) of the title compound are obtained.

### Diastereomer 61-1, Racemic:

HPLC (method 2): Rₜ=4.46 min.

MS (ESI): m/z=516 [M+H]⁺.

### Diastereomer 61-2, Racemic:

HPLC (method 2): Rₜ=4.61 min.

¹H-NMR (400 MHz, DMSO-d₆): δ=1.19 (t, 3H), 1.23 (s, 3H), 1.39 (s, 3H), 3.04 (dd, 1H), 3.24 (dd, 1H), 3.39 (s, 3H), 3.64 (dd, 2H), 3.82 (s, 3H), 4.09 (q, 2H), 4.57 (dd, 1H), 5.37 (s, 1H), 6.57 (d, 1H), 7.08-7.24 (m, 3H), 7.74 (dd, 1H), 8.02 (d, 1H).

HPLC (method 2): Rₜ=4.72 min.

MS (ESI): m/z=516 [M+H]⁺.

### Example 62

### [8-Chloro-6-(2,3-dimethoxyphenyl)-1-(2-hydroxy-1,1-dimethylethyl)-4H,6H-[1,2,4]triazolo[4,3-a]-[4,1]benzoxazepin-4-yl]acetic acid (racemic diastereomer)

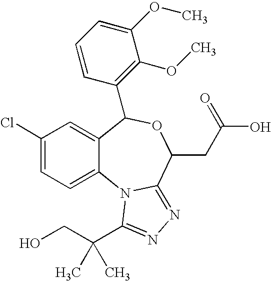

42 mg (0.08 mmol) of the compound from Example 61-2 are dissolved in 2 ml of dioxane, and 240 μl of 1 N hydrochloric acid are added. The mixture is stirred at 80° C. for 18 hours, the solvent is removed in vacuo, and the residue is washed with 20 ml of diethyl ether. 36 mg (91% of theory) of the title compound are obtained.

### Diastereomer 62-1, Racemic:

¹H-NMR (300 MHz, DMSO-d₆): δ=1.23 (s, 3H), 1.40 (s, 3H), 3.00 (dd, 1H), 3.20 (dd, 1H), 3.39 (s, 3H), 3.64 (dd, 2H), 3.82 (s, 3H), 4.54 (dd, 1H), 5.38 (s, 1H), 6.57 (d, 1H), 7.11-7.25 (m, 3H), 7.73 (dd, 1H), 8.02 (d, 1H).

HPLC (method 2): Rₜ=4.26 min.

MS (ESI): m/z=488 [M+H]⁺.

The racemic diastereomer 62-1 is separated into its enantiomers by chromatography [column: KBD 5326B, 250 mm×30 mm, based on the selector poly(N-methacryloyl-L-leucinedicyclopropylmethylamide); eluent: isohexane/ethyl acetate 20:80; flow rate: 25 ml/min.; oven: 22° C.; UV detection: 254 nm].

### Enantiomer 62-2:

HPLC (column: KBD 5326B, 250 mm×4.6 mm; eluent: isohexane/ethyl acetate 20:80; flow rate: 1 ml/min.; oven: 22° C.; UV detection: 254 nm): Rₜ=6.92 min.

¹H-NMR (400 MHz, DMSO-d₆): δ=1.23 (s, 3H), 1.38 (s, 3H), 2.99 (dd, 1H), 3.20 (dd, 1H), 3.39 (s, 3H), 3.58-3.70 (m, 2H), 3.81 (s, 3H), 4.53 (dd, 1H), 5.15 (t, 1H), 5.34 (s, 1H), 6.57 (d, 1H), 7.11-7.23 (m, 3H), 7.72 (dd, 1H), 8.02 (d, 1H), 12.47 (broad, 1H).

HPLC (method 2): Rₜ=4.14 min.

MS (ESI): m/z=488.3 and 490.3 [M+H]⁺.

### Enantiomer 62-3:

HPLC (column: KBD 5326B, 250 mm×4.6 mm; eluent: isohexane/ethyl acetate 20:80; flow rate: 1 ml/min.; oven: 22° C.; UV detection: 254 nm): Rₜ=11.13 min.

### Example 63

### Ethyl [8-chloro-6-(2,3-dimethoxyphenyl)-1-(hydroxymethyl)-4H,6H-[1,2,4]triazolo[4,3-a][4,1]benzoxazepin-4-yl]acetate

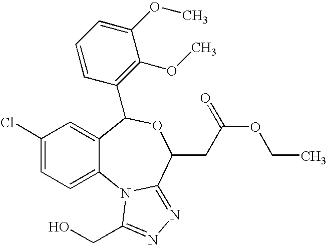

770 mg (1.77 mmol) of the compound from Example 8A and 318 mg (3.53 mmol) of 2-hydroxyacetyl hydrazide are mixed with 7.7 ml of dioxane and stirred under reflux for 4 days. Then a further 7.7 ml of dioxane and 318 mg (3.53 mmol) of 2-hydroxyacetyl hydrazide are added. The mixture is again stirred under reflux for 2 days. The residue after removal of the solvent in vacuo is purified by preparative HPLC (eluent: acetonitrile/water, gradient 20:80→80:20), with the resulting diastereomers being separated from one another. 103 mg (12% of theory) of diastereomer 63-1 are obtained.

### Diastereomer 63-1, Racemic:

¹H-NMR (300 MHz, DMSO-d₆): δ=1.18 (t, 3H), 3.04 (dd, 1H), 3.25-3.28 (m, 1H), 3.32 (s, 3H), 3.81 (s, 3H), 4.09 (q, 2H), 4.60 (dd, 1H), 4.86 (dd, 1H), 4.96 (dd, 1H), 5.48 (s, 1H), 5.87 (t, OH), 6.64 (d, 1H), 7.11-7.26 (m, 3H), 7.76 (dd, 1H), 8.04 (d, 1H).

HPLC (method 1): Rₜ=4.50 min.

MS (ESI): m/z=474 [M+H]⁺.

### Example 64

### [8-Chloro-6-(2,3-dimethoxyphenyl)-1-(hydroxymethyl)-4H,6H-[1,2,4]triazolo[4,3-a][4,1]benzoxazepin-4-yl]acetic acid (racemic diastereomer)

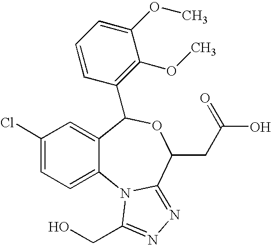

80 mg (0.17 mmol) of the compound from Example 63-1 are dissolved in 2.5 ml of dioxane, and 150 μl of concentrated hydrochloric acid are added. The mixture is stirred at 80° C. for 18 hours and then a further 501 of concentrated hydrochloric acid are added, and the mixture is stirred at 80° C. for a further 2 days. The solvent is then removed in vacuo, and the residue is purified by preparative HPLC (eluent: acetonitrile/water, gradient 20:80→80:20). 43 mg (46% of theory, 81% purity) of the title compound are obtained.

HPLC (method 2): Rₜ=3.97 min.

MS (ESI): m/z=446 [M+H]⁺.

### Example 65

### [8-Chloro-6-(2,3-dimethoxyphenyl)-4-(2-oxo-2-piperidin-1-ylethyl)-4H,6H-[1,2,4]triazolo[4,3-a]-[4,1]benzoxazepin-1-yl]methanol (racemic diastereomer)

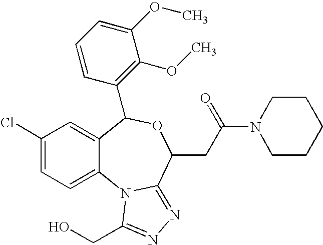

51 mg of PyBOP (0.10 mmol) and 11 mg of N,N-diisopropylethylamine (0.10 mmol) are added to 40 mg (0.09 mmol) of the compound from Example 64 in 1.8 ml of N,N-dimethylformamide. After stirring at room temperature for 1 h, 8 mg of piperidine (0.10 mmol) are added. After stirring at room temperature for 16 h, the solvent is removed under reduced pressure. The residue is purified by preparative HPLC (eluent: acetonitrile/water, gradient 20:80→80:20). 4 mg (8% of theory) of the title compound are obtained.

¹H-NMR (400 MHz, DMSO-d₆): δ=1.39-1.57 (m, 8H), 3.03-3.09 (m, 1H), 3.25-3.28 (m, 1H), 3.32 (s, 3H), 3.36-3.48 (m, 2H), 3.81 (s, 3H), 4.60 (dd, 1H), 4.85 (dd, 1H), 4.95 (dd, 1H), 5.43 (s, 1H), 5.85 (s, 1H), 6.65 (d, 1H), 7.12-7.25 (m, 3H), 7.76 (dd, 1H), 8.04 (d, 1H).

HPLC (method 2): Rₜ=4.45 min.

MS (ESI): m/z=513 [M+H]⁺.

### Example 66

### Ethyl [8-chloro-6-(2,3-dimethoxyphenyl)-1-(piperidin-1-ylmethyl)-4H,6H-[1,2,4]triazolo[4,3-a][4 μl]benzoxazepin-4-yl]acetate

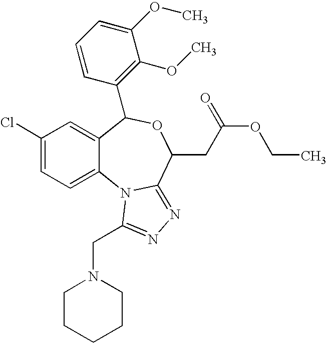

620 mg (1.42 mmol) of the compound from Example 8A and 447 mg (2.85 mmol) of 2-piperidin-1-ylacetyl hydrazide are mixed with 5 ml of dioxane and stirred under reflux for 22 hours. The residue after removal of the solvent in vacuo is purified by preparative HPLC (eluent: acetonitrile/water, gradient 20:80→80:20), with the resulting diastereomers being separated from one another. 52 mg (6% of theory) of the diastereomer 66-1 are obtained.

### Diastereomer 66-1 Racemic:

¹H-NMR (400 MHz, DMSO-d₆): δ=1.18 (t, 3H), 1.27-1.42 (m, 6H), 2.31-2.37 (m, 4H), 3.05 (dd, 1H), 3.25-3.30 (m, 1H), 3.32 (s, 3H), 3.67 (dd, 1H), 3.81 (s, 3H), 4.02 (dd, 1H), 4.07-4.13 (m, 2H), 4.83 (dd, 1H), 5.46 (s, 1H), 6.60 (d, 1H), 7.11-7.25 (m, 3H), 7.72 (dd, 1H), 8.10 (d, 1H).

HPLC (method 2): Rₜ=4.56 min.

MS (ESI): m/z=541 [M+H]⁺.

### Example 67

### Ethyl [8-chloro-6-(2,3-dimethoxyphenyl)-1-(morpholin-4-ylmethyl)-4H,6H-[1,2,4]triazolo[4,3-a][4,1]benzoxazepin-4-yl]acetate (racemic diastereomer)

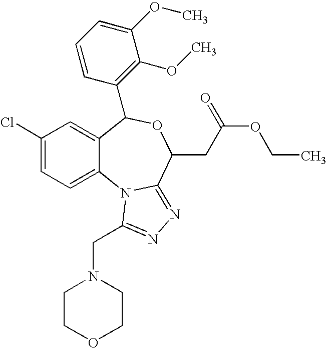

620 mg (1.42 mmol) of the compound from Example 8A and 453 mg (2.85 mmol) of 2-morpholin-4-ylacetyl hydrazide are mixed with 5 ml of dioxane and stirred under reflux for 22 hours. The residue after removal of the solvent in vacuo is purified by preparative HPLC (eluent: acetonitrile/water, gradient 20:80→80:20). 84 mg (11% of theory) of the title compound are obtained.

¹H-NMR (400 MHz, DMSO-d₆): δ=1.18 (t, 3H), 2.32-2.43 (m, 4H), 3.09 (dd, 1H), 3.18-3.30 (m, 3H), 3.32 (s, 3H), 3.39-3.42 (m, 2H), 3.70-3.77 (m, 1H), 3.82 (s, 3H), 4.05-4.15 (m, 3H), 4.84 (dd, 1H), 5.46 (s, 1H), 6.60 (d, 1H), 7.12-7.25 (m, 3H), 7.72 (dd, 1H), 8.10 (d, 1H).

HPLC (method 2): Rₜ=4.43 min.

MS (ESI): m/z=543 [M+H]⁺.

### Example 68

### [8-Chloro-6-(2,3-dimethoxyphenyl)-1-(morpholin-4-ylmethyl)-4H,6H-[1,2,4]triazolo[4,3-a][4,1]-benzoxazepin-4-yl]acetic acid (racemic diastereomer)

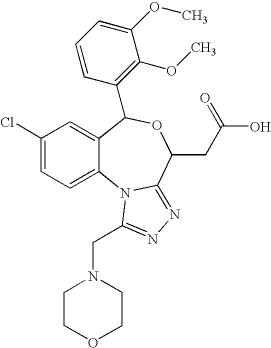

56 mg (0.10 mmol) of the compound from Example 67 are dissolved in 2.5 ml of dioxane, and 150 μl of concentrated hydrochloric acid are added. The mixture is stirred at 80° C. for 22 hours. The solvent is then removed in vacuo, and the residue is washed with diethyl ether and purified by preparative HPLC (eluent: acetonitrile/water, gradient 20:80→80:20). 30 mg (56% of theory) of the title compound are obtained.

¹H-NMR (300 MHz, DMSO-d₆): δ=2.35-2.48 (m, 4H), 3.09 (dd, 1H), 3.18-3.30 (m, 3H), 3.32 (s, 3H), 3.39-3.42 (m, 2H), 3.75 (d, 1H), 3.82 (s, 3H), 4.08 (d, 1H), 4.85 (dd, 1H), 5.44 (s, 1H), 6.60 (d, 1H), 7.12-7.25 (m, 3H), 7.72 (dd, 1H), 8.10 (d, 1H).

HPLC (method 2): Rₜ=4.01 min.

MS (ESI): m/z=515 [M+H]⁺.

### Example 69

### [1-(3-Aminopropyl)-8-chloro-6-(2,3-dimethoxyphenyl)-4H,6H-[1,2,4]triazolo[4,3-a][4,1]benzoxazepin-4-yl]acetic acid (racemic diastereomer)

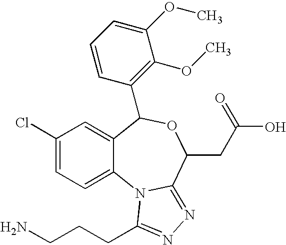

200 mg (0.33 mmol) of the compound from Example 14A are dissolved in 6 ml of dioxane, and 100 g of concentrated hydrochloric acid are added. The mixture is stirred at 80° C. for 22 hours. 100 μl of concentrated hydrochloric acid are again added, and the mixture is stirred at 80° C. for a further 22 hours. The solvent is then removed in vacuo, and the residue is purified by preparative HPLC (eluent: acetonitrile/water, gradient 20:80→80:20). 83 mg (53% of theory) of the title compound are obtained.

¹H-NMR (300 MHz, DMSO-d₆): δ=1.84-1.91 (m, 2H), 2.78-2.87 (m, 4H), 3.13 (dd, 1H), 3.36 (s, 3H), 3.36-3.42 (m, 1H), 3.81 (s, 3H), 4.76 (dd, 1H), 5.35 (s, 1H), 6.63 (d, 1H), 7.12-7.25 (m, 3H), 7.73 (dd, 1H), 7.98 (d, 1H).

HPLC (method 1): Rₜ=3.94 min.

MS (ESI): m/z=473 [M+H]⁺.

### Example 70

### Ethyl [1-[2-(allyloxy)ethyl]-8-chloro-6-(2,3-dimethoxyphenyl)-4H,6H-[1,2,4]triazolo[4,3-a][4,1]-benzoxazepin-4-yl]acetate (racemic diastereomer)

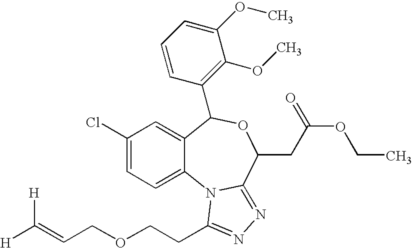

600 mg (1.38 mmol) of the compound from Example 8A and 397 mg (2.75 mmol) of the compound from Example 16A are mixed with 20 ml of dioxane and stirred under reflux for 3 days. The residue after removal of the solvent in vacuo is purified by preparative HPLC (eluent: acetonitrile/water, gradient 20:80→80:20). 120 mg (15% of theory) of the title compound are obtained.

¹H-NMR (400 MHz, DMSO-d₆): δ=1.18 (t, 3H), 3.07 (dd, 1H), 3.20-3.28 (m, 3H), 3.36 (s, 3H), 3.72 (t, 2H), 3.81 (s, 3H), 3.89-3.91 (m, 2H), 4.09 (q, 2H), 4.79 (dd, 1H), 5.09 (dd, 1H), 5.16 (dd, 1H), 5.43 (s, 1H), 5.78 (ddt, 1H), 6.64 (d, 1H), 7.11-7.24 (m, 3H), 7.74 (dd, 1H), 7.94 (d, 1H).

HPLC (method 2): Rₜ=4.94 min.

MS (ESI): m/z=528 [M+H]⁺.

### Example 71

### [8-Chloro-6-(2,3-dimethoxyphenyl)-1-(2-hydroxyethyl)-4H,6H-[1,2,4]triazolo[4,3-a][4,1]benzoxazepin-4-yl]acetic acid (racemic diastereomer)

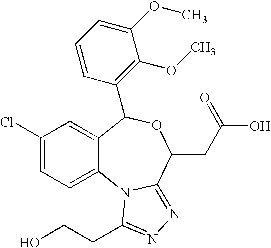

105 mg (0.20 mmol) of the compound from Example 70 are dissolved in 5 ml of dioxane and 597 μl of 1 N hydrochloric acid are added. The mixture is stirred at 80° C. for 2 days. The residue after removal of the solvent is dissolved in 4 ml of acetic acid, and 69 mg (0.06 mmol) of tetrakis-(triphenylphosphine)palladium(0) and 21 mg (0.30 mmol) of pyrrolidine are added. The mixture is stirred at room temperature for 22 hours and then the solvent is removed in vacuo. The residue is purified by preparative HPLC (eluent: acetonitrile/water, gradient 20:80→80:20). 16 mg (17% of theory) of the title compound are obtained.

¹H-NMR (400 MHz, CDCl₃): δ=3.01-3.05 (m, 1H), 3.20-3.27 (m, 3H), 3.45 (s, 3H), 3.86 (s, 3H), 4.08-4.16 (m, 2H), 4.90 (dd, 1H), 5.57 (s, 1H), 6.82-6.84 (m, 1H), 6.97 (d, 1H), 7.16-7.26 (m, 3H), 7.46-7.50 (m, 1H).

HPLC (method 2): Rₜ=4.02 min.

MS (ESI): m/z=460 [M+H]⁺.

### Example 72

### Ethyl [8-chloro-1-isopropyl-6-(2-methoxyphenyl)-4H,6H-[1,2,4]triazolo[4,3-a][4,1]benzoxazepin-4-yl]acetate (racemic diastereomer)

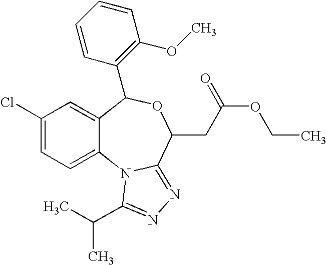

100 mg (0.25 mmol) of the compound from Example 22A and 50 mg (0.49 mmol) of 2-methylpropanoyl hydrazide are mixed with 1.5 ml of dioxane and stirred under reflux for 2 days. The residue after removal of the solvent in vacuo is purified by preparative HPLC (eluent: acetonitrile/water, gradient 20:80→80:20). 15 mg (12% of theory) of the title compound are obtained.

¹H-NMR (400 MHz, DMSO-d₆): δ=0.97 (d, 3H), 1.18 (t, 3H), 1.50 (d, 3H), 3.09 (dd, 1H), 3.23-3.29 (m, 1H), 3.29 (s, 3H), 3.45-3.50 (m, 1H), 4.10 (q, 2H), 4.78 (dd, 1H), 5.31 (s, 1H), 6.60 (d, 1H), 7.02 (d, 1H), 7.11 (dd, 1H), 7.41 (dd, 1H), 7.52 (d, 1H), 7.74 (dd, 1H), 7.94 (d, 1H).

HPLC (method 1): Rₜ=4.98 min.

MS (ESI): m/z=456 [M+H]⁺.

### Example 73

### Ethyl [8-chloro-6-(2,3-dihydro-1,4-benzodioxin-5-yl)-1-isopropyl-4H,6H-[1,2,4]triazolo[4,3-a][4 μl]benzoxazepin-4-yl]acetate (racemic diastereomer)

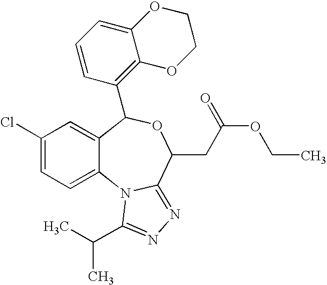

47 mg (0.11 mmol) of the compound from Example 28A and 22 mg (0.22 mmol) of 2-methylpropanoyl hydrazide are mixed with 0.7 ml of dioxane and stirred under reflux for 2 days. The residue after removal of the solvent in vacuo is purified by preparative HPLC (eluent: acetonitrile/water, gradient 20:80→80:20). 16 mg (31% of theory) of the title compound are obtained.

¹H-NMR (400 MHz, DMSO-d₆): δ=0.96 (d, 3H), 1.18 (t, 3H), 1.50 (d, 3H), 3.07 (dd, 1H), 3.25 (dd, 1H), 3.46 (tt, 1H), 3.92-3.98 (m, 2H), 4.03-4.15 (m, 4H), 4.76 (dd, 1H), 5.28 (s, 1H), 6.71 (d, 1H), 6.91-7.00 (m, 2H), 7.07 (d, 1H), 7.74 (dd, 1H), 7.93 (d, 1H).

HPLC (method 2): Rₜ=4.89 min.

MS (ESI): m/z=484 [M+H]⁺.

### Example 74

### 4-{[8-Chloro-6-(2,3-dimethoxyphenyl)-1-isopropyl-4H,6H-[1,2,4]triazolo[4,3-a][4,1]benzoxazepin-4-yl]acetyl}piperazin-2-one (racemic diastereomer)

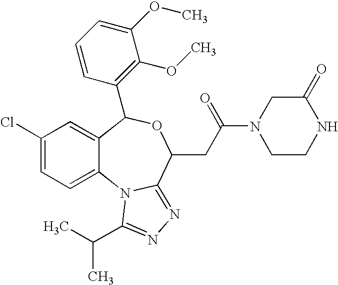

150 mg of PyBOP (0.29 mmol) and 37 mg of N,N-diisopropylethylamine (0.29 mmol) are added to 120 mg (0.26 mmol) of the compound from Example 3 in 2.4 ml of N,N-dimethylformamide. After stirring at room temperature for 1 h, 29 mg of piperazinone (0.29 mmol) are added. After stirring at room temperature for 5 h, the solvent is removed under reduced pressure. The residue is purified by preparative HPLC (eluent: acetonitrile/water, gradient 20:80→80:20). 11 mg (8% of theory) of the title compound are obtained.

¹H-NMR (400 MHz, DMSO-d₆): δ=0.98 (d, 3H), 1.51 (d, 3H), 3.08-3.20 (m, 2H), 3.37-3.50 (m, 4H), 3.50-3.72 (m, 3H), 3.82 (s, 3H), 4.74-4.82 (m, 1H), 5.23 (s, 1H), 6.63 (d, 1H), 7.13-7.23 (m, 3H), 7.74 (d, 1H), 7.96 (d, 1H).

HPLC (method 2): Rₜ=4.14 min.

MS (ESI): m/z=484 [M+H]⁺.

### Example 75

### Ethyl (4-{[8-chloro-6-(2,3-dimethoxyphenyl)-1-isopropyl-4H,6H-[1,2,4]triazolo[4,3a][4,1]benzoxazepin-4-yl]acetyl}piperazin-1-yl)acetate

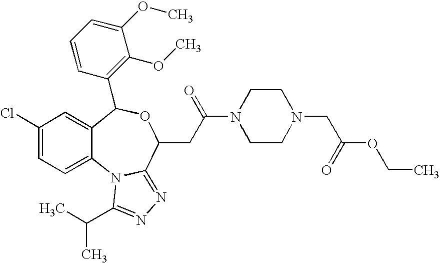

118 mg of PyBOP (0.23 mmol) and 29.3 mg of N,N-diisopropylethylamine (0.23 mmol) are added to 80.0 mg (0.18 mmol) of the compound from Example 3 (stereoisomer 3-3) in 5 ml of tetrahydrofuran. After stirring at room temperature for 0.5 h, 39.1 mg of 1-(ethoxycarbonylmethyl)piperazine (0.23 mmol) are added. After stirring at RT for 18 h, the solvent is removed under reduced pressure. The residue is purified by preparative HPLC (eluent: acetonitrile/water, gradient 10:90→95:5). 80 mg (75% of theory) of the title compound are obtained.

¹H-NMR (400 MHz, CD₃CN): δ=1.06 (d, 3H), 1.22 (t, 3H), 1.55 (d, 3H), 2.46-2.63 (m, 4H), 3.12 (dd, 1H), 3.21 (s, 2H), 3.36 (dd, 1H), 3.39 (s, 3H), 3.44 (m, 1H), 3.48-3.62 (m, 4H), 3.83 (s, 3H), 4.11 (q, 2H), 4.87 (t, 1H), 5.43 (s, 1H), 6.71 (d, 1H), 7.08 (t, 1H), 7.21 (d, 2H), 7.59 (dd, 1H), 7.65 (d, 1H).

LC/MS (method 4): Rₜ=2.08 min., m/z=612 [M+H]⁺.

### Example 76

### 4-{[8-Chloro-6-(2,3-dimethoxyphenyl)-1-isopropyl-4H,6H-[1,2,4]triazolo[4,3-a][4,1]benzoxazepin-4-yl]acetyl}piperazin-1-yl)acetic acid

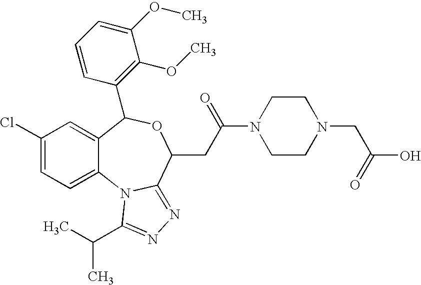

30 mg (0.05 mmol) of the compound from Example 75 are dissolved in 1 ml of dioxane, mixed with 0.1 ml of concentrated hydrochloric acid and stirred at 60° C. for 30 h. The solvent is removed under reduced pressure, and the residue is purified by preparative HPLC (eluent: acetonitrile/water with 0.1% formic acid, gradient 5:95→95:5). 9 mg (33% of theory) of the title compound are obtained.

¹H-NMR (400 MHz, DMSO-d₆): δ=0.99 (d, 3H), 1.52 (d, 3H), 3.17 (dd, 1H), 3.33 (s, 3H), 3.42-3.53 (m, 6H), 3.66-3.75 (m, 4H), 3.81 (s, 3H), 4.17 (s, 2H), 4.80 (t, 1H), 5.33 (s, 1H), 6.64 (d, 1H), 7.12-7.28 (m, 3H), 7.75 (dd, 1H), 7.97 (d, 1H).

LC/MS (method 5): Rₜ=1.92 min., m/z=584 [M+H]⁺.

### Example 77

### Ethyl 4-({[8-chloro-6-(2,3-dimethoxyphenyl)-1-isopropyl-4H,6H-[1,2,4]triazolo[4,3-a][4,1]benzoxazepin-4-yl]acetyl}amino)butyrate

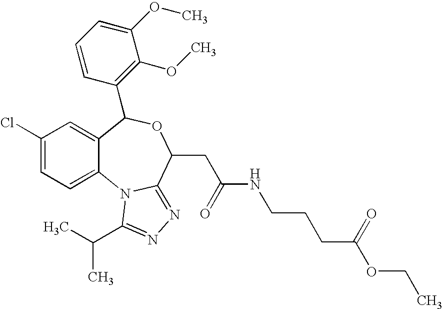

125 mg of PyBOP (0.240 mmol) and 42 μl of N,N-diisopropylethylamine (31 mg, 0.240 mmol) are added to 100 mg of the compound from Example 3 (0.218 mmol) in 3 ml of tetrahydrofuran and 100 μl of N,N-dimethylformamide at 0° C. The mixture is stirred at RT for 1 h and then 22 μl of ethyl 4-aminobutyrate are added (32 mg, 0.240 mmol). The mixture is stirred at RT for 1 h and then the solvent is removed under reduced pressure. The residue is purified by preparative HPLC (eluent: acetonitrile/water with 0.1% formic acid, gradient 10:90→95:5). 24 mg (17% of theory) of the title compound are obtained.

¹H-NMR (300 MHz, DMSO-d₆): δ=1.00 (d, 3H), 1.17 (t, 3H), 1.52 (d, 3H), 1.67 (tt, 2H), 2.30 (t, 2H), 2.89-3.09 (m, 3H), 3.16 (m, 1H), 3.34 (s, 3H), 3.49 (m, 1H), 3.81 (s, 3H), 4.06 (q, 2H), 4.76 (dd, 1H), 5.82 (s, 1H), 6.61 (d, 1H), 7.09-7.23 (m, 3H), 7.72 (dd, 1H), 7.92 (d, 1H), 8.09 (t, 1H).

HPLC (method 2): Rₜ=4.61 min.

MS (ESI): m/z=571.3 [M+H]⁺.

### Example 78

### N-{[8-Chloro-6-(2,3-dimethoxyphenyl)-1-isopropyl-4H,6H-[1,2,4]triazolo[4,3-a][4,1]benzoxazepin-4-yl]acetyl}-β-alanine ethyl ester (racemic diastereomer)

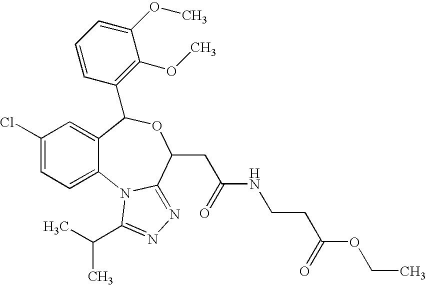

125 mg of PyBOP (0.240 mmol) and 42 μl of N,N-diisopropylethylamine (31 mg, 0.240 mmol) are added to 100 mg of the compound from Example 3 (0.218 mmol) in 3 ml of tetrahydrofuran and 100 μl of N,N-dimethylformamide at 0° C. The mixture is stirred at RT for 1 h and then 28 mg of ethyl 3-aminopropionate (0.240 mmol) are added. The mixture is stirred at RT for 1 h and then the solvent is removed under reduced pressure. The residue is purified by preparative HPLC (eluent: acetonitrile/water with 0.1% formic acid, gradient 10:90→95:5). 16 mg (12% of theory) of the title compound are obtained.

¹H-NMR (400 MHz, DMSO-d₆): δ=1.00 (d, 3H), 1.19 (t, 3H), 1.52 (d, 3H), 2.42-2.57 (m, overlapped by DMSO signal, 2H), 2.92 and 2.98 (AB signal, additionally split to d, 2H), 3.27 and 3.50 (2 m, AB signal, 2H), 3.30 (s, 3H), 3.83 (s, 3H), 4.06 (q, 2H), 4.57 (dd, 1H), 5.32 (s, 1H), 6.62 (d, 1H), 7.09-7.17 (m, 2H), 7.18-7.26 (m, 1H), 7.75 (dd, 1H), 7.95 (d, 1H), 8.24 (t, 1H).

LC/MS (method 3): Rₜ=2.25 min., m/z=557 [M+H]⁺.

### Example 79

### 2-{8-Chloro-6-(2,3-dimethoxyphenyl)-4-[2-(1,1-dioxothiomorpholin-4-yl)-2-oxoethyl]-4H,6H-[1,2,4]triazolo[4,3-a][4,1]benzoxazepin-1-yl}-2-methylpropan-1-ol

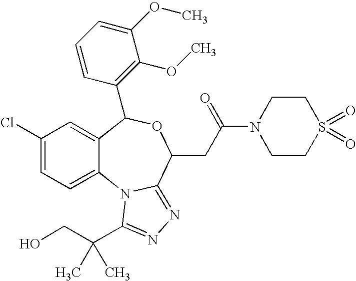

142 mg (0.27 mmol) of PyBOP and 35 mg (0.27 mmol) of N,N-diisopropylethylamine are successively added to a solution of 70 mg (0.14 mmol) of the compound from Example 62 (stereoisomer 62-2) in 5 ml of anhydrous tetrahydrofuran at room temperature. After 30 minutes, 37 mg (0.27 mmol) of thiomorpholine S,S-dioxide are added, and the mixture is stirred overnight. It is then evaporated to dryness, and the residue is purified by preparative HPLC. 38 mg (44% of theory) of a white solid are obtained.

¹H-NMR (300 MHz, DMSO-d₆): δ=1.22 (s, 3H), 1.38 (s, 3H), 3.03 (m, 1H), 3.18 (dd, 1H), 3.38 (s, 3H), 3.48 (s, 3H), 3.63 (m, 2H), 3.81 (s, 3H), 3.98 (m, 2H), 4.60 (dd, 1H), 5.21 (t, 1H), 5.33 (s, 1H), 6.59 (d, 1H), 7.12-7.27 (m, 3H), 7.73 (dd, 1H), 8.03 (d, 1H).

HPLC (method 2): Rₜ=4.20 min.

MS (ESI): m/z=605 and 607 [M+H]⁺.

The following compounds are prepared in analogy to the examples described above from the appropriate starting compounds:

### Example 80

### [1-[(1S)-1-Aminoethyl]-8-chloro-6-(2,3-dimethoxyphenyl)-4H,6H-[1,2,4]triazolo[4,3-a][4,1]benzoxazepin-4-yl]acetic acid hydrochloride

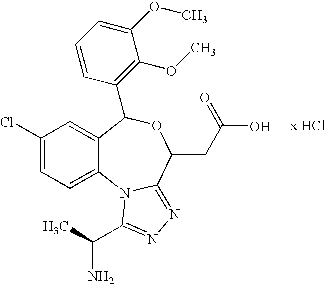

### Example 81

### 1-{[8-Chloro-6-(2,3-dimethoxyphenyl)-1-(2-hydroxy-1,1-dimethylethyl)-4H,6H-[1,2,4]triazolo[4,3-a][4,1]benzoxazepin-4-yl]acetyl}pyrrolidin-3-ol

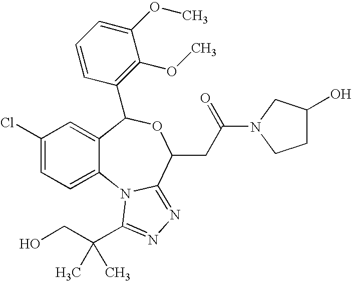

### Example 82

### 2-{8-Chloro-6-(2,3-dimethoxyphenyl)-4-[2-(1,1-dioxothiomorpholin-4-yl)-2-oxoethyl]-4H,6H-[1,2,4]triazolo[4,3-a][4,1]benzoxazepin-1-yl}-2-methylpropan-1-amine

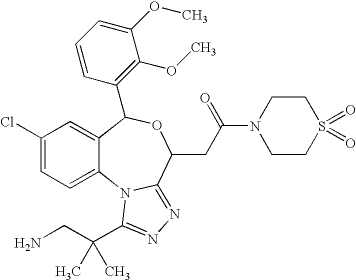

### Example 83

### 1-{[1-(2-Amino-1,1-dimethylethyl)-8-chloro-6-(2,3-dimethoxyphenyl)-4H,6H-[1,2,4]triazolo[4,3-a][4,1]benzoxazepin-4-yl]acetyl}pyrrolidin-3-ol

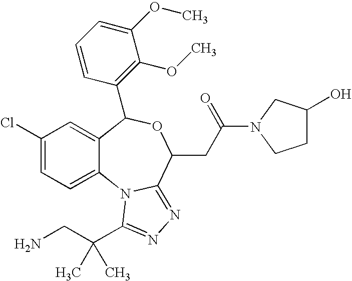

### Example 84

### 1-{[8-Chloro-6-(2,3-dimethoxyphenyl)-1-(1-methoxy-1-methylethyl)-4H,6H-[1,2,4]triazolo[4,3-a]-[4,1]benzoxazepin-4-yl]acetyl}pyrrolidin-3-ol

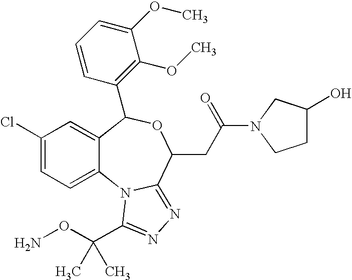

### Example 85

### Ethyl [8-chloro-6-(2,3-dimethoxyphenyl)-1-(2-methoxy-1,1-dimethylethyl)-4H,6H-[1,2,4]triazolo[4,3-a][4,1]benzoxazepin-4-yl]acetate

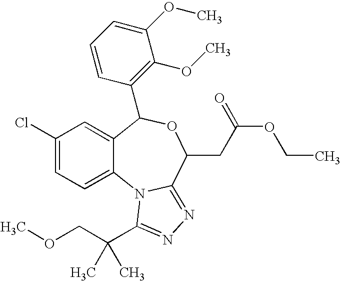

### Example 86

### [8-Chloro-6-(2,3-dimethoxyphenyl)-1-(2-methoxy-1,1-dimethylethyl)-4H,6H-[1,2,4]triazolo[4,3-a][4,1]benzoxazepin-4-yl]acetic acid

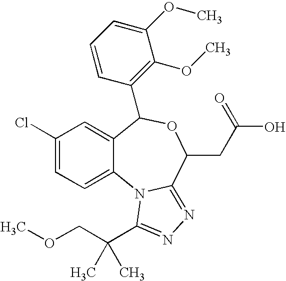

### Example 87

### 1-{[8-Chloro-6-(2,3-dimethoxyphenyl)-1-(2-methoxy-1,1-dimethylethyl)-4H,6H-[1,2,4]triazolo[4,3-a][4,1]benzoxazepin-4-yl]acetyl}pyrrolidin-3-ol

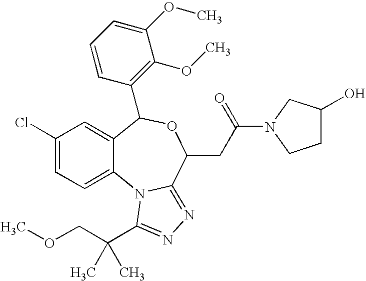

### Example 88

### 1-{[8-Chloro-6-(2,3-dimethoxyphenyl)-1-isopropyl-4H,6H-[1,2,4]triazolo[4,3-a][4,1]benzoxazepin-4-yl]acetyl}pyrrolidine-3,4-diol

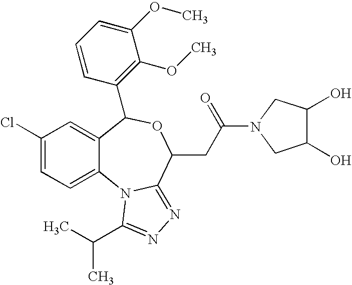

### Example 89

### Ethyl [1-(2-amino-1,1-dimethylethyl)-8-chloro-6-(2,3-dimethoxyphenyl)-4H,6H-[1,2,4]triazolo[4,3-a][4,1]benzoxazepin-4-yl]acetate

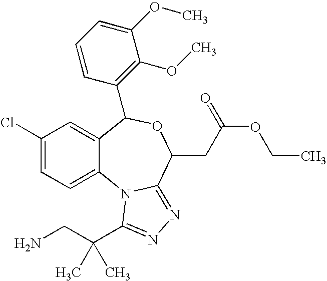

55.0 mg (0.09 mmol) of the compound from Example 30A-2 are dissolved in 2 ml of dioxane, mixed with 0.1 ml of concentrated hydrochloric acid and stirred at 80° C. for 20 h. The solvent is removed under reduced pressure and the residue is purified by preparative HPLC (eluent: acetonitrile/water, gradient 10:90→95:5). 11 mg (24% of theory) of the title compound are obtained.

¹H-NMR (300 MHz, DMSO-d₆): δ=1.18 (s, 3H and t, 3H), 1.61 (s, 3H), 3.12 (d, 1H), 3.13 and 3.27 (AB signal, additionally split as d, 2H), 3.36 (s, 3H), 3.58 (d, 1H), 3.82 (s, 3H), 4.11 (q, 2H), 4.61 (t, 1H), 5.58 (s, 1H), 6.58 (d, 1H), 7.09-7.25 (m, 3H), 7.76 (dd, 1H), 7.88 (dd, 1H), 8.02 (br. s, 3H).

LC/MS (method 4): Rₜ=1.95 min., m/z=515 [M+H]⁺.

### Example 90

### [1-(2-Amino-1,1-dimethylethyl)-8-chloro-6-(2,3-dimethoxyphenyl)-4H,6H-[1,2,4]triazolo[4,3-a]-[4,1]benzoxazepin-4-yl]acetic acid

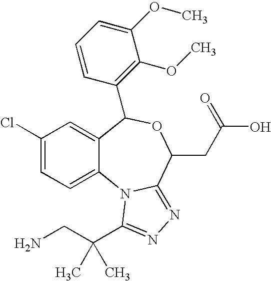

135 mg (0.22 mmol) of the compound from Example 30A-2 are dissolved in 5 ml of dioxane, mixed with 0.5 ml of concentrated hydrochloric acid and stirred at 80° C. for 40 h. The solvent is removed under reduced pressure, and the residue is purified by preparative HPLC (eluent: acetonitrile/water, gradient 10:90→95:5). 32 mg (28% of theory) of the title compound are obtained.

¹H-NMR (300 MHz, DMSO-d₆): δ=1.19 (s, 3H), 1.59 (s, 3H), 3.01 and 3.22 (AB signal, additionally split as d, 2H), 3.15 (d, 1H), 3.41 (s, 3H), 3.53 (d, 1H), 3.82 (s, 3H), 4.56 (t, 1H), 5.55 (s, 1H), 6.58 (d, 1H), 7.09-7.25 (m, 3H), 7.75 (dd, 1H), 7.88 (dd, 1H), 8.02 (br. s, 3H).

LC/MS (method 3): Rₜ=1.47 min., m/z=486 [M+H]⁺.

### B. ASSESSMENT OF THE PHARMACOLOGICAL ACTIVITY

The pharmacological effect of the compounds according to the invention can be shown in the following assays:

### 1. Squalene Synthase Inhibition Assay

### a) Obtaining Microsomes:

Microsomes from rat livers are prepared as source of squalene synthase for the activity assay. The rat livers are comminuted and homogenized in twice the volume of homogenization buffer [100 mM Tris/HCl, 0.2 M sucrose, 30 mM nicotinamide, 14 mM sodium fluoride, 5 mM dithiothreitol, 5 mM MgCl₂, protease inhibitor cocktail (from Sigma, Taufkirchen), pH 7.5] (Dounce homogenizer). The supernatant from a 10 000 g centrifugation is then centrifuged at 100 500 g. The pelleted microsomes are taken up in homogenization buffer, diluted to 10 mg/ml protein and stored at −80° C.

### b) Squalene Synthase Activity Assay:

The conversion of trans,trans-[1-³H]-farnesyl pyrophosphate into [³H]-squalene by the microsomal squalene synthase takes place under the following reaction conditions: rat liver microsomes (protein content 65 μg/ml), 1 mM NADPH, 6 mM glutathione, 10% PBS, 10 mM sodium fluoride, 5 mM MgCl₂, pH 7.5. The compound to be tested in each case is dissolved in DMSO and added to the assay in a defined concentration. The reaction is started by adding farnesyl pyrophosphate (final concentration 5 μM) and 20 kBq/ml trans,trans-[1-³H]-farnesyl pyrophosphate, and is incubated at 37° C. for 10 min. Subsequently, 100 μl of the reaction solution are mixed with 200 μl of chloroform, 200 μl of methanol and 60 μl of 5 N sodium hydroxide solution and adjusted to 2 mM squalene. After vigorous mixing and subsequent phase separation, an aliquot of the organic phase is transferred into scintillation fluid (Packard Ultima Gold LSC Cocktail) and the organically extractable radioactive compounds are quantified (LS 6500, from Beckman). The reduction in the radioactive signal is directly proportional to the inhibition of squalene synthase by the compound employed in each case.

The exemplary embodiments show IC₅₀ values of <10 μM in this assay.

### 2. Inhibition of Squalene and Cholesterol Synthesis in the Liver of Mice

Male NMRI mice are kept on normal rodent diet (NAFAG 3883) in metabolism cages. The light/dark cycle comprises 12 hours, from 06.00 to 18.00 and from 18.00 to 06.00. The animals are employed with a body weight of between 25 g and 40 g in groups of 8-10 animals in the experiments. Feed and drinking water are available to the animals ad libitum.

The substances are, according to their solubility, administered orally in aqueous tragacanth suspension (0.5%) or in Solutol HS15/saline solution (20:80) by gavage in a volume of 10 ml/kg of body weight or else injected subcutaneously in Solutol HS15/saline solution (20:80) or DMSO/saline solution (20:80). The corresponding control groups receive only the corresponding formulating agent without active substance. One or two hours after administration of the substance, the animals receive intraperitoneal injections of radiolabelled ¹⁴C-mevalonolactone. One or two hours after the ¹⁴C-mevalonolactone injection, or 24 hours after the administration of substance, the animals are sacrificed, the abdominal cavity is opened, and liver tissue is removed. Immediately after removal, the tissue is dried on the surface, weighed and homogenized in isopropanol. The further processing and extraction of the synthesized squalene and its secondary products takes place by a method of I. Duncan et al. (J. Chromatogr. 1979, 162), modified by H. Bischoff et al. (Atherosclerosis 1997, 135).

The extracted lipid fraction is taken up in 1 ml of isopropanol, transferred into scintillation vials, made up with 15 ml of Ultima Gold® scintillation fluid (Packard) and counted in a liquid scintillation counter (Beckmann Coulter LS 6500).

After calculation of the specific ¹⁴C activity of the lipid fraction (dpm/g of liver tissue), the rate of synthesis of the radiolabelled ¹⁴C squalene and of the ¹⁴C secondary metabolites of the animals treated with the active substance is compared with the rate of synthesis of the radiolabelled ¹⁴C squalene and of the ¹⁴C secondary metabolites of the control animals treated only with formulating agent. A reduction in the rate of synthesis by ≧30% compared with the rate of synthesis for the control animals (=100%) is regarded as pharmacologically active if the statistical assessment by Student's t test results in a p value of <0.05.

TABLE 1
Inhibition of sterol biosynthesis in mice

| | | Inhibition relative |
|---|---|---|
| Example | Dose | to the untreated control group |
| 3 | 3 mg/kg p.o. | 81% |
| 41 | 3 mg/kg p.o. | 81% |
| 50 | 3 mg/kg p.o. | 75% |
| 62-2 | 3 mg/kg p.o. | 77% |

### 3. Inhibition of Squalene and Cholesterol Synthesis in the Liver of Rats

Male Wistar rats are kept on normal rodent diet (NAFAG 3883) in Makrolon® type III cages. The light/dark cycle comprises 12 hours, from 06.00 to 18.00 and from 18.00 to 06.00. The animals are employed with a body weight of between 150 g and 200 g in groups of 6-8 animals in the experiments. The feed is withdrawn from the animals 18-22 hours before the start of the experiment; drinking water is available ad libitum up to the end of the experiment.

The substances are, according to their solubility, administered orally in aqueous tragacanth suspension (0.5%) or in Solutol HS15/saline solution (20:80) by gavage in a volume of 10 ml/kg of body weight or else injected subcutaneously in Solutol HS15/saline solution (20:80) or DMSO/saline solution (20:80). The corresponding control groups receive only the corresponding formulating agent without active substance. One or two hours after administration of the substance, the animals receive intraperitoneal injections of radiolabelled ¹⁴C-mevalonolactone. One or two hours after the ¹⁴C-mevalonolactone injection, or 24 hours after the administration of substance, the animals are sacrificed, the abdominal cavity is opened, and liver tissue is removed. Immediately after removal, the tissue is dried on the surface, weighed and homogenized in isopropanol. The further processing and extraction of the synthesized squalene and its secondary products takes place by a method of I. Duncan et al. (J. Chromatogr. 1979, 162), modified by H. Bischoff et al. (Atherosclerosis 1997, 135).

The extracted lipid fraction is taken up in 1 ml of isopropanol, transferred into scintillation vials, made up with 15 ml of Ultima Gold® scintillation fluid (Packard) and counted in a liquid scintillation counter (Beckmann Coulter LS 6500).

After calculation of the specific ¹⁴C activity of the lipid fraction (dpm/g of liver tissue), the rate of synthesis of the radiolabelled ¹⁴C squalene and of the ¹⁴C secondary metabolites of the animals treated with the active substance is compared with the rate of synthesis of the radiolabelled ¹⁴C squalene and of the ¹⁴C secondary metabolites of the control animals treated only with formulating agent. A reduction in the rate of synthesis by ≧30% compared with the rate of synthesis for the control animals (=100%) is regarded as pharmacologically active if the statistical assessment by Student's t test results in a p value of <0.05.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

A mixture of compound according to the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. The stirring process is continued until the compound according to the invention has completely dissolved.

i.v. solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A compound of the formula (I) 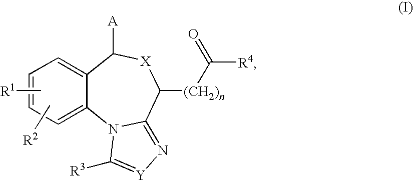
in which
A is (C₆-C₁₀)-aryl or 5- to 10-membered heteroaryl, each of which may be substituted up to three times, identically or differently, by substituents selected from the group consisting of halogen, cyano, nitro, trifluoromethyl, hydroxy, fluoromethoxy, trifluoromethoxy, (C₁-C₆)-alkyl, (C₁-C₆)-alkoxy, amino, and mono- and di-(C₁-C₆)-alkylamino,
or
A is a group of the formula 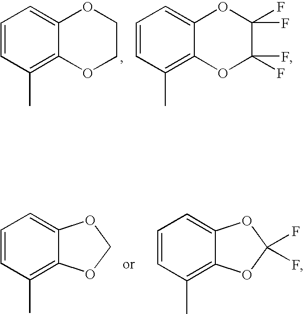
X is O, S or N—R⁵ in which
R⁵ is hydrogen or (C₁-C₆)-alkyl,
Y is N or C—R⁶ in which
R⁶ is hydrogen, hydroxy or (C₁-C₆)-alkyl,
n is the number 1, 2 or 3,
R¹ and R² are identical or different and are independently of one another hydrogen, halogen, cyano, nitro, trifluoromethyl, trifluoromethoxy, (C₁-C₆)-alkyl or (C₁-C₆)-alkoxy,
R³ is (C₁-C₈)-alkyl, (C₂-C₈)-alkenyl, (C₂-C₈)-alkynyl, each of which may be substituted by (C₃-C₈)-cycloalkyl, or is (C₃-C₈)-cycloalkyl, where
(C₁-C₈)-alkyl, (C₂-C₈)-alkenyl, (C₂-C₈)-alkynyl and (C₃-C₈)-cycloalkyl may each be substituted by hydroxy, (C₁-C₆)-alkoxy, (C₂-C₆)-alkenoxy, (C₁-C₆)-acyloxy, amino, or mono- or di-(C₁-C₆)-alkylamino or by a 4- to 8-membered saturated heterocycle which is linked via an N atom and which may comprise a further heteroatom selected from O and S,
and
R⁴ is a group of the formula —OR⁷ or —NR⁸R⁹ in which
R⁷ is hydrogen or (C₁-C₆)-alkyl,
R⁸ and R⁹ are identical or different and are independently of one another hydrogen, (C₁-C₆)-alkyl or (C₃-C₈)-cycloalkyl, each of which may be substituted by substituents selected from the group consisting of carboxyl, (C₁-C₆)-alkoxycarbonyl, aminocarbonyl, and mono- and di-(C₁-C₆)-alkylaminocarbonyl,
or
R⁸ and R⁹ form together with the nitrogen atom to which they are bonded a 4- to 8-membered heterocycle which may comprise a further ring member selected from the series consisting of N—R¹⁰, O, S, SO and SO₂ and may be substituted by substituents selected from the group consisting of hydroxy, oxo, amino, (C₁-C₆)-alkyl, carboxyl, (C₁-C₆)-alkoxycarbonyl, aminocarbonyl, and mono- and di-(C₁-C₆)-alkylaminocarbonyl, in which
(C₁-C₆)-alkyl in turn may be substituted by substituents selected from the group consisting of hydroxy, amino, carboxyl, (C₁-C₆)-alkoxycarbonyl, aminocarbonyl, and mono- and di-(C₁-C₆)-alkylaminocarbonyl, and
R¹⁰ is hydrogen, (C₁-C₄)-alkyl, (C₁-C₄)-acyl or (C₁-C₄)-alkoxycarbonyl in which
(C₁-C₄)-alkyl may in turn be substituted by carboxyl or (C₁-C₄)-alkoxycarbonyl,
or a salt, solvate, or solvate of a salt thereof.

2. The compound of the formula (I) according to claim 1, in which
A is phenyl, naphthyl or pyridyl, each of which may be substituted up to twice, identically or differently, by substituents selected from the group consisting of fluorine, chlorine, bromine, cyano, nitro, trifluoromethyl, fluoromethoxy, trifluoromethoxy, (C₁-C₄)-alkyl, (C₁-C₄)-alkoxy, amino, and mono- and di-(C₁-C₄)-alkylamino,
or
A is a group of the formula 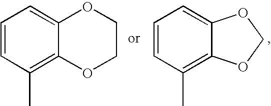
X is O,
Y is N or C—R⁶ in which
R⁶ is hydrogen, hydroxy or (C₁-C₄)-alkyl,
n is the number 1, 2 or 3,
R¹ and R² are identical or different and are independently of one another hydrogen, fluorine, chlorine, bromine, cyano, nitro, trifluoromethyl, trifluoromethoxy, (C₁-C₄)-alkyl or (C₁-C₄)-alkoxy,
R³ is (C₁-C₆)-alkyl which may be substituted by (C₃-C₆)-cycloalkyl, or is (C₃-C₆)-cycloalkyl, where
(C₁-C₆)-alkyl and (C₃-C₆)-cycloalkyl may each be substituted by hydroxy, (C₁-C₄)-alkoxy or amino,
and
R⁴ is a group of the formula —OR⁷ or —NR⁸R⁹, in which
R⁷ is hydrogen or (C₁-C₆)-alkyl,
R⁸ and R⁹ are identical or different and are independently of one another hydrogen, (C₁-C₆)-alkyl or (C₃-C₆)-cycloalkyl, each of which may be substituted by substituents selected from the group consisting of carboxyl, (C₁-C₆)-alkoxycarbonyl, aminocarbonyl, and mono- and di-(C₁-C₆)-alkylaminocarbonyl,
or
R⁸ and R⁹ form together with the nitrogen atom to which they are bonded a 5- to 7-membered heterocycle which may comprise a further ring member selected from the series consisting of N—R¹⁰, O, S and SO₂ and may be substituted by substituents selected from the group consisting of hydroxy, oxo, amino, (C₁-C₆)-alkyl, carboxyl, (C₁-C₆)-alkoxycarbonyl, aminocarbonyl, and mono- and di-(C₁-C₆)-alkylaminocarbonyl, in which
(C₁-C₆)-alkyl in turn may be substituted by substituents selected from the group consisting of hydroxy, amino, carboxyl, (C₁-C₆)-alkoxycarbonyl, aminocarbonyl, and mono- and di-(C₁-C₆)-alkylaminocarbonyl, and
R¹⁰ is hydrogen, (C₁-C₄)-alkyl, (C₁-C₄)-acyl or (C₁-C₄)-alkoxycarbonyl in which
(C₁-C₄)-alkyl in turn may be substituted by carboxyl or (C₁-C₄)-alkoxycarbonyl,
or a salt, solvate, or solvate of a salt thereof.

3. The compound of the formula (I) according to claim 1 or 2, in which
A is phenyl which is substituted once or twice, identically or differently, by fluorine, chlorine, bromine, methyl, methoxy, ethoxy, fluoromethoxy or dimethylamino,
X is O,
Y is N,
n is the number 1,
R¹ and R² are independently of one another hydrogen or chlorine,
R³ is (C₁-C₆)-alkyl or (C₃-C₆)-cycloalkyl, each of which may be substituted by hydroxy, (C₁-C₄)-alkoxy or amino,
and
R⁴ is a group of the formula —OR⁷ or —NR⁸R⁹ in which
R⁷ is hydrogen or (C₁-C₄)-alkyl,
R⁸ and R⁹ are identical or different and are independently of one another hydrogen or (C₁-C₄)-alkyl which may be substituted by carboxyl or (C₁-C₄)-alkoxycarbonyl,
or
R⁸ and R⁹ form together with the nitrogen atom to which they are bonded a 5- or 6-membered heterocycle which may comprise a further ring member selected from the series consisting of N—R¹⁰, O, S and SO₂ and may be substituted by substituents selected from the group consisting of hydroxy, oxo, amino, (C₁-C₄)-alkyl, carboxyl, (C₁-C₄)-alkoxycarbonyl, aminocarbonyl, and mono- and di-(C₁-C₄)-alkylaminocarbonyl, in which
(C₁-C₄)-alkyl in turn may be substituted by substituents selected from the group consisting of hydroxy, amino, carboxyl, (C₁-C₄)-alkoxycarbonyl, aminocarbonyl, mono- and di-(C₁-C₄)-alkylaminocarbonyl, and
R¹⁰ is hydrogen, (C₁-C₄)-alkyl or (C₁-C₄)-acyl,
or a salt, solvate, or solvate of a salt thereof.

4. A compound of the formula (I-A) 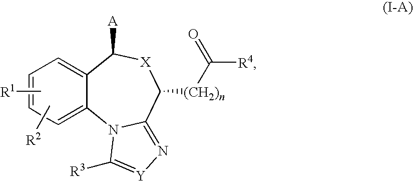
in which
A, X, Y, n, R¹, R², R³ and R⁴ each have the meanings indicated in claim 1,
or a salt, solvate, or solvate of a salt thereof.

5. A compound of the formula (I-B) 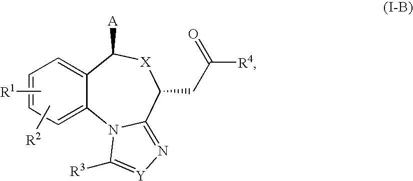
in which
A, Y, R¹, R², R³ and R⁴ each have the meanings indicated in claim 1,
or a salt, solvate, or solvate of a salt thereof.

6. A process for preparing a compound of the formula (I), (I-A) or (I-B) as defined in claims 1-3, 4, and 5, respectively, characterized in that a compound of the formula (II) 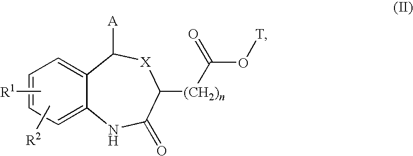
in which R¹, R², A, X and n each have the meanings indicated in claims 1-3, 4, and 5, respectively, and
T is (C₁-C₄)-alkyl,
is first converted in an inert solvent with a suitable sulphurizing agent into a compound of the formula (III) 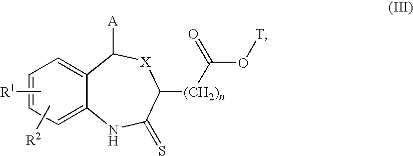
in which R¹, R², A, T, X and n each have the abovementioned meanings, subsequently reacted in an inert solvent with a compound of the formula (IV) 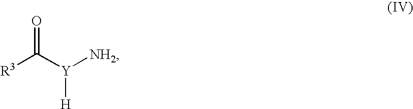
in which Y and R³ each have the meanings indicated in claims 1-3, 4, and 5, respectively,
with cyclization to give a compound of the formula (V) 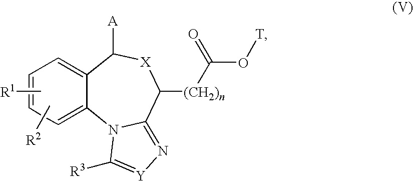
in which R¹, R², R³, A, T, X, Y and n each have the abovementioned meanings, the latter is hydrolysed under acidic conditions to a carboxylic acid of the formula (VI) 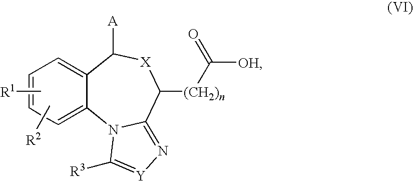
in which R¹, R², R³, A, X, Y and n each have the abovementioned meanings, and then converted by methods known from the literature for the esterification or amidation of carboxylic acids into the compound of the formula (I) and the compound of the formula (I) is where appropriate separated into the stereochemically pure isomers and/or reacted with the appropriate (i) solvent and/or (ii) base or acid to give the solvate, salt and/or solvate of the salt thereof.

7. (canceled)

8. (canceled)

9. A pharmaceutical composition comprising a compound as defined in any of claims 1 to 5 in combination with a further active ingredient selected from the group consisting of cholesterol-lowering statins, cholesterol absorption inhibitors, HDL-elevating, triglyceride-lowering and/or apolipoprotein B-lowering substances, oxidation inhibitors and compounds having antiinflammatory activity.

10. A pharmaceutical composition comprising a compound as defined in any of claims 1 to 5 in combination with an inert, non-toxic, pharmaceutically suitable excipient.

11. (canceled)

12. A method for the treatment and/or prevention of dyslipidaemias, arteriosclerosis, restenosis and ischaemias in humans and animals comprising administering an effective amount of at least one compound as defined in any of claims 1 to 5, or of a pharmaceutical composition as defined in claim 9 or 10.

13. The process of claim 6 in which the sulphurizing agent is diphosphorus pentasulphide.
